SEQUENCE LISTING (58 sequences)

SEQ ID NO: 1 - DNA, 6354 bp, Homo sapiens
gatcttggttcactgcaacctccgccccgcaggttcaagggattctcctgcctcagcctccggagtagctgggattacaggtgcctgccaccccacccagctaatttttgtatttttagtagaaacggggtttcaccatattggccaggctggtctcgaactccagacctcaggtgatccacccgcctcggcctcccaaagtgctgggatcacatggtgtgagccaccgccgcccggcctcttttttttttttttttttttttttttttttgagatagtctccctctgttgtccaggctggagtacagtggcatgatctcggctcactgcaacctctgcctcctgggtcaagcaattctcttgcctcagcctcctgagtggctgggattacaggctagcgccaccacactgggctaatttttgtatttttagtagagatggggtttcaccatcttggccaggctggtctcgaactcttgacctcaagtgatccggcctcccaaagtgctgggattacagacgtgagccactgcgcccggcctatttatcttgattactgagttttttggcaccctctcaaattctgtcccagaagtcagtatctcactcatctcacccatgtcctggctgcttagagattgtctgggaagtggagaccctccccagctccctcctccttctccagggccaaagacagcaaggaatagggcgactgtttacatcagctcctgtcctagagatcccctgagattggacagctgagcccgtggagagggattccccacccccaagtaggcaaccacccactctcctccagagggaacgagggatccatctcagttctcatccttgaccactcccctccagctctgattgtataacctgaaacccacacacggtttgtaactttttttttttttttccgagacggagtctcgctctgtttccaggctggagtgcagtggcgcgatcttggctcactgcaatctccgcctcccgggttcaagcgattcccctggctcagcctccctagtagctgggactacagacgcgcaccaccatgcccggctaatttgtttgtaacctttttaacctgtctggcttttccacagggagaggatttgcatatgctgtgtcctgtgtgatgaaaggagccctttttacacaccccctctttcaatgcactcagacttcaaagcccatcactcattcattcaacaaacacccactaagcacctcttgaacccggttcaactatctcccctctaggcactcacccaacctcgggcactctccgcgcctcctctgagctcctcccgcgtcccatacagcccctgctcactgctggaaaatattcctgttaagctccgtctccccaccagtcctgcttgctttctgtgtgtctgggataggttgcaaaactggaggtgacaaatgtgggcaggaaatggagggctcccttatactcctagggtcggcctcagccctgccatttcctgcccaataccgcggatgcaactggcatgggacccggaagtgtgtggtaaagcgccggggcttcgcctggccgcccctcccggacgcttgcctgcagtcagcgaagcttttccaacccaggtctgggccctgcgagctccaggtgcctctgccccgcggcgctggcgaagtcgaagcccgagaacgcccaccgcagcgatgcgaaggccgcttctggggtggggctgaggctgcagctgcctccctttcgcaccaaggaccccaaccttcagcgacgcagccgccgctcccgcccaggctgggaggcactgtagggatccgacgcttcaggtggccaaagagcgactttctctgattccagggccctggcggggcaggctccagtaccgcactcggcggaggccgaaggtctgtggggcaggacaggagttcctcgtgccgtcggaagggccgaggacgaaggagggcgctaattcactccccactgggctggcggcaacgccgaatcccgcagtgaccgcggagggccaaggtgaaaactgctgggggcgccgagggcaggtgtggggaggggcggctccagggagcaaggagcccacctgcctcgccgccgcagctgccctgggtcgaccgcccacgctttcccctgggtcacgacctccggatccaactgtgcccggctcctgccccttcccctttgctgctgctgcgcgggctgtaacttgacgtctaggctggggcgtccagggcgcagtctccgctcaggtcgccagcgctccactcgctccactgggccacagacccgctggcgctggggtcccgctggcgctggggtcccgctggcgctggggtcccgctggcgctggggtcccgctggcgctggggtcccgctggcgctggggtcccgctggcgctggggtcccgcggttgtgcacgtgagtccgcgtggccccgtcccgcgcggttgtgcacgtgagtccgcgcggccgcgtcccgccccgctccagggagccagcgcgccgccacctgggatgttaggactctcgttgtgttctttggactgctttgggggatttcggcgtacccctaggatctaggagttttggaagttgtttgagagaaaccagctctgggagggcctcgcatccagtctcctgtctcggcttcggaccggggttccgggttaaggtgctcagaggaacagctgatccttctacccttgcgcagggcagagatccccaaacctctttctaaaatgcagggtcttagtctttctcagggagtcagtgaacctagactcccagtctcttgagcccaagcatgaatagggaactggggaccaccaccatgctcatatttctggtggccaggaagtccaggcaggcttttgcccactgcagacggatctctttcttcaggggtcaagaaaggctctgtacagtaagccaaccaagccccaccagcagagctgcgctgcaaccaggttcctagtgtctttcccacactcccagttccaagcgacacaggacacagccccaattccacctccccccacccacttcctgccctgttgcctcatcttccctgctacacactccaagtcatccacccaacccccagctcctggtaactcagccccagctcctggaagtccagccccagccctgctaccaccacttccaccttggcctcccctgagaacaagtggagggcaaatagagcccaggcttgaattcactcgtttgaaaaacaactcaagccaaaccctgtgggagcagtgcctgcctggggcacctcaggctcatgtgcacctctgaattctttcgggcacctgccccaaatgtaaagaggctgctacccaatgcctgggcccctctgactctctgctcctctctggctgtctctctgtttttgtcgtctctcaccttcccctgtcatggccagtacttggtcctcaggccagaaaaggtggatcccgtgtccctggattttactaatgccaggagtcacataaatactcctatatatacacagagagggcccgcgcatgcttggaaatcctcagataccaaagaacaaagtgcaggacacaacagagatactgtacatattgagaaatgtgtacatcccccaaatgcagagaaatgcacaccaacatacagagacacacctcacaaacacattcacagaaacagacacactcacactccaagaaatactaagacacccatgaagggaacatttcagacatgcacaggaccctgaactcacatggacacagacacccaggagcaggtgggccctgactcaggtgcacacagcctaacacacagtactcacacacaagctcccctaagtctaaatgccgcaagagactcccacagaaagaaaattcccccagaggcctccaaggccctgcctggaaggaagaggaagaaagtgcccgccaggcaccgaaatgctaaggcactgtaaagtgaaaatcatttccccaactccgtgggaacagcacttccattcccatctcttagggaaacagggaagtggtttaactcgttctgcaaatcaactccagatccacaagagtgccctccccctcggcggggagaggccaggctcccagtgctgcagcccagtgaatgctgatccgctgaggcccaccccaggccacagggtgctgggcaaactcaccaagcccgcacgtctgcctaacatctggctgggccaggaagagtgcctggtggacacccagcctggcgacacacaggaggcacggccggtgaagaaaatggatctgtgcaccaagggcctgcaccatctcgccgggctctgccctgacagaccattccttacctttcttgatcacggactggtctaggaggctcattcccggctcgtccattgcctgcaaatagcagagggcagcggtgagggcgcataaagccctagccgcccatgtcttcccaagcccctgccacccagggtctctgtctgctccgcccaaggcgcaccagcttcgcttggggcggcgacgggcaggcccaggaatcgctatgtccaggcactgagctggagccctgggcatctcactgtgcagtgcaaaaagggaattttgaatcttacactggtaactattatgcttctcttcgccccaggtgcaggctgccccatggatgccacacgtggcagcggggagaaaagaagtgattcggtccctgccgagggttgctgccacccacgcactgctggcggactcagtgccaggcagccttgatggccaggcaacgtctaacccaggaggtgggacacatcctcggagtcaccagtgagatgtggcgagctgggagaaaagtcggcttcctgcatttgtcaccttctgcctccagccatggtagtaaaccaccgcggccagtcccgacccatcccggatccctcgggtctcacctgcaggtcctccagaccgggggccgccgccagccccggaaggccgagaggtgcgtctgccaggccgtgcgcgccgtcggccaggccgtgcaggagccggggcacggcagtggcatagtcacggcgcgggtcaaggcccagggcgcgggcgggcggtgccagcaggccgggaggctcctcgtgggcgcgggcggctgcgcggggcgcggcccaggcggcctgaggaggctgcggctgcgccaggggcgccaggccgccatatgggtcccctgccaggtggggaaaggctgcggcggcgtcgggcgcctgaccgtagggcagcggtggctgcgggtagggcggcgggaagtagggcggctggaattcggcggcagctgtggcggccggcgtgtggcagagcgggggcgccggcccgtaggccgcctggggcagagacgacaggcgggccccgccggcagctgctcccagcccgtcggggcgctcctggggaaggagaggggtgccttaggtgacccactggcagctgaaaggaccccagcctgaaaggatcctcctcagctcgggagggttcagctgcagtccctcccgtttcttggggcccgaggtttatttccgagccacccgcgggcgcaggagcctcaggctggggacagcggtcacttgcgcatcgcgttctcagctggcgaagtccctgcggccccctccctccgagatttcgttagcgcgggacaggagggttggaaagtccagcactcaggcgccccgatcccccggccgaatatgtcccgggcccgagactactcaccatggcggagtaggtgtgcaccagcatggggcggtgagtgccgggcgcgaggcgtggcggcggcggagccctagcccgacggcgagcgctgcgggcagaggcgccgggagccggcacagcccgcggaggtcacggtgcggtagctgggtgcgacccagccgggatcctagtgggcgtggggtcctcagcggtggccgttcccgggcgggtccatggaggcaggacggacgggcactgatcgcacttggaaaagtcggtcaacgggtgcccagcgcccgcccttcctcctctgagcgtagcctgctccatgcactccagttatagtggcgggggcgcgccgggcctccgggggcgcgcattaaagagacaagctatggcagttctctcccttttccccagcggctgccacaggggaggcagtggctttggcgggatcgggtagggtgggtagggggcgtgaattcagcccacattgatcagagccgccttctgggctcgcagctcgcagctgcagtgaaacagaaatattcgaggctgggacgcaagggccctacatttgcctggctctatcacatccttgcctttaagcaaagcactattcttcgctg

SEQ ID NO: 2 - DNA, 6354 bp, Artificial Sequence
  Synthetic Construct TFAP2E CpG methylated Sense Bisulfite Sequence
gattttggtttattgtaattttcgtttcgtaggtttaagggatttttttgttttagttttcggagtagttgggattataggtgtttgttattttatttagttaatttttgtatttttagtagaaacggggttttattatattggttaggttggtttcgaattttagattttaggtgatttattcgtttcggttttttaaagtgttgggattatatggtgtgagttatcgtcgttcggttttttttttttttttttttttttttttttttttgagatagtttttttttgttgtttaggttggagtatagtggtatgatttcggtttattgtaatttttgttttttgggttaagtaatttttttgttttagttttttgagtggttgggattataggttagcgttattatattgggttaatttttgtatttttagtagagatggggttttattattttggttaggttggtttcgaatttttgattttaagtgattcggttttttaaagtgttgggattatagacgtgagttattgcgttcggtttatttattttgattattgagttttttggtatttttttaaattttgttttagaagttagtattttatttattttatttatgttttggttgtttagagattgtttgggaagtggagattttttttagtttttttttttttttttagggttaaagatagtaaggaatagggcgattgtttatattagtttttgttttagagattttttgagattggatagttgagttcgtggagagggattttttatttttaagtaggtaattatttatttttttttagagggaacgagggatttattttagtttttatttttgattatttttttttagttttgattgtataatttgaaatttatatacggtttgtaatttttttttttttttttcgagacggagtttcgttttgtttttaggttggagtgtagtggcgcgattttggtttattgtaattttcgtttttcgggtttaagcgatttttttggtttagtttttttagtagttgggattatagacgcgtattattatgttcggttaatttgtttgtaatttttttaatttgtttggtttttttatagggagaggatttgtatatgttgtgttttgtgtgatgaaaggagttttttttatatatttttttttttaatgtatttagattttaaagtttattatttatttatttaataaatatttattaagtattttttgaattcggtttaattattttttttttaggtatttatttaatttcgggtatttttcgcgttttttttgagtttttttcgcgttttatatagtttttgtttattgttggaaaatatttttgttaagtttcgtttttttattagttttgtttgttttttgtgtgtttgggataggttgtaaaattggaggtgataaatgtgggtaggaaatggagggtttttttatatttttagggtcggttttagttttgttattttttgtttaatatcgcggatgtaattggtatgggattcggaagtgtgtggtaaagcgtcggggtttcgtttggtcgtttttttcggacgtttgtttgtagttagcgaagtttttttaatttaggtttgggttttgcgagttttaggtgtttttgtttcgcggcgttggcgaagtcgaagttcgagaacgtttatcgtagcgatgcgaaggtcgtttttggggtggggttgaggttgtagttgtttttttttcgtattaaggattttaatttttagcgacgtagtcgtcgttttcgtttaggttgggaggtattgtagggattcgacgttttaggtggttaaagagcgattttttttgattttagggttttggcggggtaggttttagtatcgtattcggcggaggtcgaaggtttgtggggtaggataggagtttttcgtgtcgtcggaagggtcgaggacgaaggagggcgttaatttattttttattgggttggcggtaacgtcgaatttcgtagtgatcgcggagggttaaggtgaaaattgttgggggcgtcgagggtaggtgtggggaggggcggttttagggagtaaggagtttatttgtttcgtcgtcgtagttgttttgggtcgatcgtttacgtttttttttgggttacgattttcggatttaattgtgttcggtttttgtttttttttttttgttgttgttgcgcgggttgtaatttgacgtttaggttggggcgtttagggcgtagttttcgtttaggtcgttagcgttttattcgttttattgggttatagattcgttggcgttggggtttcgttggcgttggggtttcgttggcgttggggtttcgttggcgttggggtttcgttggcgttggggtttcgttggcgttggggtttcgttggcgttggggtttcgcggttgtgtacgtgagttcgcgtggtttcgtttcgcgcggttgtgtacgtgagttcgcgcggtcgcgtttcgtttcgttttagggagttagcgcgtcgttatttgggatgttaggattttcgttgtgttttttggattgttttgggggatttcggcgtatttttaggatttaggagttttggaagttgtttgagagaaattagttttgggagggtttcgtatttagttttttgtttcggtttcggatcggggtttcgggttaaggtgtttagaggaatagttgatttttttatttttgcgtagggtagagatttttaaatttttttttaaaatgtagggttttagttttttttagggagttagtgaatttagatttttagttttttgagtttaagtatgaatagggaattggggattattattatgtttatatttttggtggttaggaagtttaggtaggtttttgtttattgtagacggatttttttttttaggggttaagaaaggttttgtatagtaagttaattaagttttattagtagagttgcgttgtaattaggtttttagtgttttttttatatttttagttttaagcgatataggatatagttttaattttatttttttttatttattttttgttttgttgttttattttttttgttatatattttaagttatttatttaatttttagtttttggtaatttagttttagtttttggaagtttagttttagttttgttattattatttttattttggttttttttgagaataagtggagggtaaatagagtttaggtttgaatttattcgtttgaaaaataatttaagttaaattttgtgggagtagtgtttgtttggggtattttaggtttatgtgtatttttgaattttttcgggtatttgttttaaatgtaaagaggttgttatttaatgtttgggtttttttgattttttgtttttttttggttgtttttttgtttttgtcgttttttatttttttttgttatggttagtatttggtttttaggttagaaaaggtggatttcgtgtttttggattttattaatgttaggagttatataaatatttttatatatatatagagagggttcgcgtatgtttggaaatttttagatattaaagaataaagtgtaggatataatagagatattgtatatattgagaaatgtgtatattttttaaatgtagagaaatgtatattaatatatagagatatattttataaatatatttatagaaatagatatatttatattttaagaaatattaagatatttatgaagggaatattttagatatgtataggattttgaatttatatggatatagatatttaggagtaggtgggttttgatttaggtgtatatagtttaatatatagtatttatatataagtttttttaagtttaaatgtcgtaagagatttttatagaaagaaaatttttttagaggtttttaaggttttgtttggaaggaagaggaagaaagtgttcgttaggtatcgaaatgttaaggtattgtaaagtgaaaattatttttttaatttcgtgggaatagtatttttatttttattttttagggaaatagggaagtggtttaattcgttttgtaaattaattttagatttataagagtgttttttttttcggcggggagaggttaggtttttagtgttgtagtttagtgaatgttgattcgttgaggtttattttaggttatagggtgttgggtaaatttattaagttcgtacgtttgtttaatatttggttgggttaggaagagtgtttggtggatatttagtttggcgatatataggaggtacggtcggtgaagaaaatggatttgtgtattaagggtttgtattatttcgtcgggttttgttttgatagattattttttattttttttgattacggattggtttaggaggtttattttcggttcgtttattgtttgtaaatagtagagggtagcggtgagggcgtataaagttttagtcgtttatgtttttttaagtttttgttatttagggtttttgtttgtttcgtttaaggcgtattagtttcgtttggggcggcgacgggtaggtttaggaatcgttatgtttaggtattgagttggagttttgggtattttattgtgtagtgtaaaaagggaattttgaattttatattggtaattattatgttttttttcgttttaggtgtaggttgttttatggatgttatacgtggtagcggggagaaaagaagtgattcggtttttgtcgagggttgttgttatttacgtattgttggcggatttagtgttaggtagttttgatggttaggtaacgtttaatttaggaggtgggatatattttcggagttattagtgagatgtggcgagttgggagaaaagtcggttttttgtatttgttattttttgtttttagttatggtagtaaattatcgcggttagtttcgatttatttcggatttttcgggttttatttgtaggttttttagatcgggggtcgtcgttagtttcggaaggtcgagaggtgcgtttgttaggtcgtgcgcgtcgtcggttaggtcgtgtaggagtcggggtacggtagtggtatagttacggcgcgggttaaggtttagggcgcgggcgggcggtgttagtaggtcgggaggtttttcgtgggcgcgggcggttgcgcggggcgcggtttaggcggtttgaggaggttgcggttgcgttaggggcgttaggtcgttatatgggttttttgttaggtggggaaaggttgcggcggcgtcgggcgtttgatcgtagggtagcggtggttgcgggtagggcggcgggaagtagggcggttggaattcggcggtagttgtggcggtcggcgtgtggtagagcgggggcgtcggttcgtaggtcgtttggggtagagacgataggcgggtttcgtcggtagttgtttttagttcgtcggggcgtttttggggaaggagaggggtgttttaggtgatttattggtagttgaaaggattttagtttgaaaggattttttttagttcgggagggtttagttgtagtttttttcgttttttggggttcgaggtttattttcgagttattcgcgggcgtaggagttttaggttggggatagcggttatttgcgtatcgcgtttttagttggcgaagtttttgcggtttttttttttcgagatttcgttagcgcgggataggagggttggaaagtttagtatttaggcgtttcgatttttcggtcgaatatgtttcgggttcgagattatttattatggcggagtaggtgtgtattagtatggggcggtgagtgtcgggcgcgaggcgtggcggcggcggagttttagttcgacggcgagcgttgcgggtagaggcgtcgggagtcggtatagttcgcggaggttacggtgcggtagttgggtgcgatttagtcgggattttagtgggcgtggggtttttagcggtggtcgttttcgggcgggtttatggaggtaggacggacgggtattgatcgtatttggaaaagtcggttaacgggtgtttagcgttcgtttttttttttttgagcgtagtttgttttatgtattttagttatagtggcgggggcgcgtcgggttttcgggggcgcgtattaaagagataagttatggtagtttttttttttttttttagcggttgttataggggaggtagtggttttggcgggatcgggtagggtgggtagggggcgtgaatttagtttatattgattagagtcgttttttgggttcgtagttcgtagttgtagtgaaatagaaatattcgaggttgggacgtaagggttttatatttgtttggttttattatatttttgtttttaagtaaagtattatttttcgttg

SEQ ID NO: 3 - DNA, 6354 bp, Artificial Sequence
  Synthetic Construct TFAP2E Sense Bisulfite Sequence non-methylated
gattttggtttattgtaatttttgttttgtaggtttaagggatttttttgttttagtttttggagtagttgggattataggtgtttgttattttatttagttaatttttgtatttttagtagaaatggggttttattatattggttaggttggttttgaattttagattttaggtgatttatttgttttggttttttaaagtgttgggattatatggtgtgagttattgttgtttggttttttttttttttttttttttttttttttttttgagatagtttttttttgttgtttaggttggagtatagtggtatgattttggtttattgtaatttttgttttttgggttaagtaatttttttgttttagttttttgagtggttgggattataggttagtgttattatattgggttaatttttgtatttttagtagagatggggttttattattttggttaggttggttttgaatttttgattttaagtgatttggttttttaaagtgttgggattatagatgtgagttattgtgtttggtttatttattttgattattgagttttttggtatttttttaaattttgttttagaagttagtattttatttattttatttatgttttggttgtttagagattgtttgggaagtggagattttttttagtttttttttttttttttagggttaaagatagtaaggaatagggtgattgtttatattagtttttgttttagagattttttgagattggatagttgagtttgtggagagggattttttatttttaagtaggtaattatttatttttttttagagggaatgagggatttattttagtttttatttttgattatttttttttagttttgattgtataatttgaaatttatatatggtttgtaattttttttttttttttttgagatggagttttgttttgtttttaggttggagtgtagtggtgtgattttggtttattgtaatttttgttttttgggtttaagtgatttttttggtttagtttttttagtagttgggattatagatgtgtattattatgtttggttaatttgtttgtaatttttttaatttgtttggtttttttatagggagaggatttgtatatgttgtgttttgtgtgatgaaaggagttttttttatatatttttttttttaatgtatttagattttaaagtttattatttatttatttaataaatatttattaagtattttttgaatttggtttaattattttttttttaggtatttatttaattttgggtattttttgtgttttttttgagttttttttgtgttttatatagtttttgtttattgttggaaaatatttttgttaagttttgtttttttattagttttgtttgttttttgtgtgtttgggataggttgtaaaattggaggtgataaatgtgggtaggaaatggagggtttttttatatttttagggttggttttagttttgttattttttgtttaatattgtggatgtaattggtatgggatttggaagtgtgtggtaaagtgttggggttttgtttggttgttttttttggatgtttgtttgtagttagtgaagtttttttaatttaggtttgggttttgtgagttttaggtgtttttgttttgtggtgttggtgaagttgaagtttgagaatgtttattgtagtgatgtgaaggttgtttttggggtggggttgaggttgtagttgttttttttttgtattaaggattttaatttttagtgatgtagttgttgtttttgtttaggttgggaggtattgtagggatttgatgttttaggtggttaaagagtgattttttttgattttagggttttggtggggtaggttttagtattgtatttggtggaggttgaaggtttgtggggtaggataggagttttttgtgttgttggaagggttgaggatgaaggagggtgttaatttattttttattgggttggtggtaatgttgaattttgtagtgattgtggagggttaaggtgaaaattgttgggggtgttgagggtaggtgtggggaggggtggttttagggagtaaggagtttatttgttttgttgttgtagttgttttgggttgattgtttatgtttttttttgggttatgatttttggatttaattgtgtttggtttttgtttttttttttttgttgttgttgtgtgggttgtaatttgatgtttaggttggggtgtttagggtgtagtttttgtttaggttgttagtgttttatttgttttattgggttatagatttgttggtgttggggttttgttggtgttggggttttgttggtgttggggttttgttggtgttggggttttgttggtgttggggttttgttggtgttggggttttgttggtgttggggttttgtggttgtgtatgtgagtttgtgtggttttgttttgtgtggttgtgtatgtgagtttgtgtggttgtgttttgttttgttttagggagttagtgtgttgttatttgggatgttaggatttttgttgtgttttttggattgttttgggggattttggtgtatttttaggatttaggagttttggaagttgtttgagagaaattagttttgggagggttttgtatttagttttttgttttggttttggattggggttttgggttaaggtgtttagaggaatagttgatttttttatttttgtgtagggtagagatttttaaatttttttttaaaatgtagggttttagttttttttagggagttagtgaatttagatttttagttttttgagtttaagtatgaatagggaattggggattattattatgtttatatttttggtggttaggaagtttaggtaggtttttgtttattgtagatggatttttttttttaggggttaagaaaggttttgtatagtaagttaattaagttttattagtagagttgtgttgtaattaggtttttagtgttttttttatatttttagttttaagtgatataggatatagttttaattttatttttttttatttattttttgttttgttgttttattttttttgttatatattttaagttatttatttaatttttagtttttggtaatttagttttagtttttggaagtttagttttagttttgttattattatttttattttggttttttttgagaataagtggagggtaaatagagtttaggtttgaatttatttgtttgaaaaataatttaagttaaattttgtgggagtagtgtttgtttggggtattttaggtttatgtgtatttttgaatttttttgggtatttgttttaaatgtaaagaggttgttatttaatgtttgggtttttttgattttttgtttttttttggttgtttttttgtttttgttgttttttatttttttttgttatggttagtatttggtttttaggttagaaaaggtggattttgtgtttttggattttattaatgttaggagttatataaatatttttatatatatatagagagggtttgtgtatgtttggaaatttttagatattaaagaataaagtgtaggatataatagagatattgtatatattgagaaatgtgtatattttttaaatgtagagaaatgtatattaatatatagagatatattttataaatatatttatagaaatagatatatttatattttaagaaatattaagatatttatgaagggaatattttagatatgtataggattttgaatttatatggatatagatatttaggagtaggtgggttttgatttaggtgtatatagtttaatatatagtatttatatataagtttttttaagtttaaatgttgtaagagatttttatagaaagaaaatttttttagaggtttttaaggttttgtttggaaggaagaggaagaaagtgtttgttaggtattgaaatgttaaggtattgtaaagtgaaaattatttttttaattttgtgggaatagtatttttatttttattttttagggaaatagggaagtggtttaatttgttttgtaaattaattttagatttataagagtgtttttttttttggtggggagaggttaggtttttagtgttgtagtttagtgaatgttgatttgttgaggtttattttaggttatagggtgttgggtaaatttattaagtttgtatgtttgtttaatatttggttgggttaggaagagtgtttggtggatatttagtttggtgatatataggaggtatggttggtgaagaaaatggatttgtgtattaagggtttgtattattttgttgggttttgttttgatagattattttttattttttttgattatggattggtttaggaggtttatttttggtttgtttattgtttgtaaatagtagagggtagtggtgagggtgtataaagttttagttgtttatgtttttttaagtttttgttatttagggtttttgtttgttttgtttaaggtgtattagttttgtttggggtggtgatgggtaggtttaggaattgttatgtttaggtattgagttggagttttgggtattttattgtgtagtgtaaaaagggaattttgaattttatattggtaattattatgtttttttttgttttaggtgtaggttgttttatggatgttatatgtggtagtggggagaaaagaagtgatttggtttttgttgagggttgttgttatttatgtattgttggtggatttagtgttaggtagttttgatggttaggtaatgtttaatttaggaggtgggatatatttttggagttattagtgagatgtggtgagttgggagaaaagttggttttttgtatttgttattttttgtttttagttatggtagtaaattattgtggttagttttgatttattttggattttttgggttttatttgtaggttttttagattgggggttgttgttagttttggaaggttgagaggtgtgtttgttaggttgtgtgtgttgttggttaggttgtgtaggagttggggtatggtagtggtatagttatggtgtgggttaaggtttagggtgtgggtgggtggtgttagtaggttgggaggttttttgtgggtgtgggtggttgtgtggggtgtggtttaggtggtttgaggaggttgtggttgtgttaggggtgttaggttgttatatgggttttttgttaggtggggaaaggttgtggtggtgttgggtgtttgattgtagggtagtggtggttgtgggtagggtggtgggaagtagggtggttggaatttggtggtagttgtggtggttggtgtgtggtagagtgggggtgttggtttgtaggttgtttggggtagagatgataggtgggttttgttggtagttgtttttagtttgttggggtgtttttggggaaggagaggggtgttttaggtgatttattggtagttgaaaggattttagtttgaaaggattttttttagtttgggagggtttagttgtagttttttttgttttttggggtttgaggtttatttttgagttatttgtgggtgtaggagttttaggttggggatagtggttatttgtgtattgtgtttttagttggtgaagtttttgtggttttttttttttgagattttgttagtgtgggataggagggttggaaagtttagtatttaggtgttttgattttttggttgaatatgttttgggtttgagattatttattatggtggagtaggtgtgtattagtatggggtggtgagtgttgggtgtgaggtgtggtggtggtggagttttagtttgatggtgagtgttgtgggtagaggtgttgggagttggtatagtttgtggaggttatggtgtggtagttgggtgtgatttagttgggattttagtgggtgtggggtttttagtggtggttgtttttgggtgggtttatggaggtaggatggatgggtattgattgtatttggaaaagttggttaatgggtgtttagtgtttgtttttttttttttgagtgtagtttgttttatgtattttagttatagtggtgggggtgtgttgggtttttgggggtgtgtattaaagagataagttatggtagtttttttttttttttttagtggttgttataggggaggtagtggttttggtgggattgggtagggtgggtagggggtgtgaatttagtttatattgattagagttgttttttgggtttgtagtttgtagttgtagtgaaatagaaatatttgaggttgggatgtaagggttttatatttgtttggttttattatatttttgtttttaagtaaagtattattttttgttg

SEQ ID NO: 4 - DNA, 6354 bp, Artificial Sequence
  Synthetic Construct TFAP2E Methylated Antisense Bisulfite Sequence
aatcttaattcactacaacctccgccccgcaaattcaaaaaattctcctacctcaacctccgaaataactaaaattacaaatacctaccaccccacccaactaatttttatatttttaataaaaacgaaatttcaccatattaaccaaactaatctcgaactccaaacctcaaataatccacccgcctcgacctcccaaaatactaaaatcacataatataaaccaccgccgcccgacctcttttttttttttttttttttttttttttttaaaataatctccctctattatccaaactaaaatacaataacataatctcgactcactacaacctctacctcctaaatcaaacaattctcttacctcaacctcctaaataactaaaattacaaactaacgccaccacactaaactaatttttatatttttaataaaaataaaatttcaccatcttaaccaaactaatctcgaactcttaacctcaaataatccgacctcccaaaatactaaaattacaaacgtaaaccactacgcccgacctatttatcttaattactaaattttttaacaccctctcaaattctatcccaaaaatcaatatctcactcatctcacccatatcctaactacttaaaaattatctaaaaaataaaaaccctccccaactccctcctccttctccaaaaccaaaaacaacaaaaaataaaacgactatttacatcaactcctatcctaaaaatcccctaaaattaaacaactaaacccgtaaaaaaaaattccccacccccaaataaacaaccacccactctcctccaaaaaaaacgaaaaatccatctcaattctcatccttaaccactcccctccaactctaattatataacctaaaacccacacacgatttataactttttttttttttttccgaaacgaaatctcgctctatttccaaactaaaatacaataacgcgatcttaactcactacaatctccgcctcccgaattcaaacgattcccctaactcaacctccctaataactaaaactacaaacgcgcaccaccatacccgactaatttatttataacctttttaacctatctaacttttccacaaaaaaaaaatttacatatactatatcctatataataaaaaaaaccctttttacacaccccctctttcaatacactcaaacttcaaaacccatcactcattcattcaacaaacacccactaaacacctcttaaacccgattcaactatctcccctctaaacactcacccaacctcgaacactctccgcgcctcctctaaactcctcccgcgtcccatacaacccctactcactactaaaaaatattcctattaaactccgtctccccaccaatcctacttactttctatatatctaaaataaattacaaaactaaaaataacaaatataaacaaaaaataaaaaactcccttatactcctaaaatcgacctcaaccctaccatttcctacccaataccgcgaatacaactaacataaaacccgaaaatatataataaaacgccgaaacttcgcctaaccgcccctcccgaacgcttacctacaatcaacgaaacttttccaacccaaatctaaaccctacgaactccaaatacctctaccccgcgacgctaacgaaatcgaaacccgaaaacgcccaccgcaacgatacgaaaaccgcttctaaaataaaactaaaactacaactacctccctttcgcaccaaaaaccccaaccttcaacgacgcaaccgccgctcccgcccaaactaaaaaacactataaaaatccgacgcttcaaataaccaaaaaacgactttctctaattccaaaaccctaacgaaacaaactccaataccgcactcgacgaaaaccgaaaatctataaaacaaaacaaaaattcctcgtaccgtcgaaaaaaccgaaaacgaaaaaaaacgctaattcactccccactaaactaacgacaacgccgaatcccgcaataaccgcgaaaaaccaaaataaaaactactaaaaacgccgaaaacaaatataaaaaaaaacgactccaaaaaacaaaaaacccacctacctcgccgccgcaactaccctaaatcgaccgcccacgctttcccctaaatcacgacctccgaatccaactatacccgactcctaccccttcccctttactactactacgcgaactataacttaacgtctaaactaaaacgtccaaaacgcaatctccgctcaaatcgccaacgctccactcgctccactaaaccacaaacccgctaacgctaaaatcccgctaacgctaaaatcccgctaacgctaaaatcccgctaacgctaaaatcccgctaacgctaaaatcccgctaacgctaaaatcccgctaacgctaaaatcccgcgattatacacgtaaatccgcgtaaccccgtcccgcgcgattatacacgtaaatccgcgcgaccgcgtcccgccccgctccaaaaaaccaacgcgccgccacctaaaatattaaaactctcgttatattctttaaactactttaaaaaatttcgacgtacccctaaaatctaaaaattttaaaaattatttaaaaaaaaccaactctaaaaaaacctcgcatccaatctcctatctcgacttcgaaccgaaattccgaattaaaatactcaaaaaaacaactaatccttctacccttacgcaaaacaaaaatccccaaacctctttctaaaatacaaaatcttaatctttctcaaaaaatcaataaacctaaactcccaatctcttaaacccaaacataaataaaaaactaaaaaccaccaccatactcatatttctaataaccaaaaaatccaaacaaacttttacccactacaaacgaatctctttcttcaaaaatcaaaaaaaactctatacaataaaccaaccaaaccccaccaacaaaactacgctacaaccaaattcctaatatctttcccacactcccaattccaaacgacacaaaacacaaccccaattccacctccccccacccacttcctaccctattacctcatcttccctactacacactccaaatcatccacccaacccccaactcctaataactcaaccccaactcctaaaaatccaaccccaaccctactaccaccacttccaccttaacctcccctaaaaacaaataaaaaacaaataaaacccaaacttaaattcactcgtttaaaaaacaactcaaaccaaaccctataaaaacaatacctacctaaaacacctcaaactcatatacacctctaaattctttcgaacacctaccccaaatataaaaaaactactacccaatacctaaacccctctaactctctactcctctctaactatctctctatttttatcgtctctcaccttcccctatcataaccaatacttaatcctcaaaccaaaaaaaataaatcccgtatccctaaattttactaataccaaaaatcacataaatactcctatatatacacaaaaaaaacccgcgcatacttaaaaatcctcaaataccaaaaaacaaaatacaaaacacaacaaaaatactatacatattaaaaaatatatacatcccccaaatacaaaaaaatacacaccaacatacaaaaacacacctcacaaacacattcacaaaaacaaacacactcacactccaaaaaatactaaaacacccataaaaaaaacatttcaaacatacacaaaaccctaaactcacataaacacaaacacccaaaaacaaataaaccctaactcaaatacacacaacctaacacacaatactcacacacaaactcccctaaatctaaataccgcaaaaaactcccacaaaaaaaaaattcccccaaaaacctccaaaaccctacctaaaaaaaaaaaaaaaaaaatacccgccaaacaccgaaatactaaaacactataaaataaaaatcatttccccaactccgtaaaaacaacacttccattcccatctcttaaaaaaacaaaaaaataatttaactcgttctacaaatcaactccaaatccacaaaaataccctccccctcgacgaaaaaaaaccaaactcccaatactacaacccaataaatactaatccgctaaaacccaccccaaaccacaaaatactaaacaaactcaccaaacccgcacgtctacctaacatctaactaaaccaaaaaaaatacctaataaacacccaacctaacgacacacaaaaaacacgaccgataaaaaaaataaatctatacaccaaaaacctacaccatctcgccgaactctaccctaacaaaccattccttacctttcttaatcacgaactaatctaaaaaactcattcccgactcgtccattacctacaaataacaaaaaacaacgataaaaacgcataaaaccctaaccgcccatatcttcccaaacccctaccacccaaaatctctatctactccgcccaaaacgcaccaacttcgcttaaaacgacgacgaacaaacccaaaaatcgctatatccaaacactaaactaaaaccctaaacatctcactatacaatacaaaaaaaaaattttaaatcttacactaataactattatacttctcttcgccccaaatacaaactaccccataaataccacacgtaacaacgaaaaaaaaaaaaataattcgatccctaccgaaaattactaccacccacgcactactaacgaactcaataccaaacaaccttaataaccaaacaacgtctaacccaaaaaataaaacacatcctcgaaatcaccaataaaatataacgaactaaaaaaaaaatcgacttcctacatttatcaccttctacctccaaccataataataaaccaccgcgaccaatcccgacccatcccgaatccctcgaatctcacctacaaatcctccaaaccgaaaaccgccgccaaccccgaaaaaccgaaaaatacgtctaccaaaccgtacgcgccgtcgaccaaaccgtacaaaaaccgaaacacgacaataacataatcacgacgcgaatcaaaacccaaaacgcgaacgaacgataccaacaaaccgaaaaactcctcgtaaacgcgaacgactacgcgaaacgcgacccaaacgacctaaaaaaactacgactacgccaaaaacgccaaaccgccatataaatcccctaccaaataaaaaaaaactacgacgacgtcgaacgcctaaccgtaaaacaacgataactacgaataaaacgacgaaaaataaaacgactaaaattcgacgacaactataacgaccgacgtataacaaaacgaaaacgccgacccgtaaaccgcctaaaacaaaaacgacaaacgaaccccgccgacaactactcccaacccgtcgaaacgctcctaaaaaaaaaaaaaaataccttaaataacccactaacaactaaaaaaaccccaacctaaaaaaatcctcctcaactcgaaaaaattcaactacaatccctcccgtttcttaaaacccgaaatttatttccgaaccacccgcgaacgcaaaaacctcaaactaaaaacaacgatcacttacgcatcgcgttctcaactaacgaaatccctacgaccccctccctccgaaatttcgttaacgcgaaacaaaaaaattaaaaaatccaacactcaaacgccccgatcccccgaccgaatatatcccgaacccgaaactactcaccataacgaaataaatatacaccaacataaaacgataaataccgaacgcgaaacgtaacgacgacgaaaccctaacccgacgacgaacgctacgaacaaaaacgccgaaaaccgacacaacccgcgaaaatcacgatacgataactaaatacgacccaaccgaaatcctaataaacgtaaaatcctcaacgataaccgttcccgaacgaatccataaaaacaaaacgaacgaacactaatcgcacttaaaaaaatcgatcaacgaatacccaacgcccgcccttcctcctctaaacgtaacctactccatacactccaattataataacgaaaacgcgccgaacctccgaaaacgcgcattaaaaaaacaaactataacaattctctcccttttccccaacgactaccacaaaaaaaacaataactttaacgaaatcgaataaaataaataaaaaacgtaaattcaacccacattaatcaaaaccgccttctaaactcgcaactcgcaactacaataaaacaaaaatattcgaaactaaaacgcaaaaaccctacatttacctaactctatcacatccttacctttaaacaaaacactattcttcgcta

SEQ ID NO: 5 - DNA, 6354 bp, Artificial Sequence
  Synthetic Construct TFAP2E Antisense Bisulfite Sequence non-methylated
aatcttaattcactacaacctccaccccacaaattcaaaaaattctcctacctcaacctccaaaataactaaaattacaaatacctaccaccccacccaactaatttttatatttttaataaaaacaaaatttcaccatattaaccaaactaatctcaaactccaaacctcaaataatccacccacctcaacctcccaaaatactaaaatcacataatataaaccaccaccacccaacctcttttttttttttttttttttttttttttttaaaataatctccctctattatccaaactaaaatacaataacataatctcaactcactacaacctctacctcctaaatcaaacaattctcttacctcaacctcctaaataactaaaattacaaactaacaccaccacactaaactaatttttatatttttaataaaaataaaatttcaccatcttaaccaaactaatctcaaactcttaacctcaaataatccaacctcccaaaatactaaaattacaaacataaaccactacacccaacctatttatcttaattactaaattttttaacaccctctcaaattctatcccaaaaatcaatatctcactcatctcacccatatcctaactacttaaaaattatctaaaaaataaaaaccctccccaactccctcctccttctccaaaaccaaaaacaacaaaaaataaaacaactatttacatcaactcctatcctaaaaatcccctaaaattaaacaactaaacccataaaaaaaaattccccacccccaaataaacaaccacccactctcctccaaaaaaaacaaaaaatccatctcaattctcatccttaaccactcccctccaactctaattatataacctaaaacccacacacaatttataactttttttttttttttccaaaacaaaatctcactctatttccaaactaaaatacaataacacaatcttaactcactacaatctccacctcccaaattcaaacaattcccctaactcaacctccctaataactaaaactacaaacacacaccaccatacccaactaatttatttataacctttttaacctatctaacttttccacaaaaaaaaaatttacatatactatatcctatataataaaaaaaaccctttttacacaccccctctttcaatacactcaaacttcaaaacccatcactcattcattcaacaaacacccactaaacacctcttaaacccaattcaactatctcccctctaaacactcacccaacctcaaacactctccacacctcctctaaactcctcccacatcccatacaacccctactcactactaaaaaatattcctattaaactccatctccccaccaatcctacttactttctatatatctaaaataaattacaaaactaaaaataacaaatataaacaaaaaataaaaaactcccttatactcctaaaatcaacctcaaccctaccatttcctacccaataccacaaatacaactaacataaaacccaaaaatatataataaaacaccaaaacttcacctaaccacccctcccaaacacttacctacaatcaacaaaacttttccaacccaaatctaaaccctacaaactccaaatacctctaccccacaacactaacaaaatcaaaacccaaaaacacccaccacaacaatacaaaaaccacttctaaaataaaactaaaactacaactacctccctttcacaccaaaaaccccaaccttcaacaacacaaccaccactcccacccaaactaaaaaacactataaaaatccaacacttcaaataaccaaaaaacaactttctctaattccaaaaccctaacaaaacaaactccaataccacactcaacaaaaaccaaaaatctataaaacaaaacaaaaattcctcataccatcaaaaaaaccaaaaacaaaaaaaaacactaattcactccccactaaactaacaacaacaccaaatcccacaataaccacaaaaaaccaaaataaaaactactaaaaacaccaaaaacaaatataaaaaaaaacaactccaaaaaacaaaaaacccacctacctcaccaccacaactaccctaaatcaaccacccacactttcccctaaatcacaacctccaaatccaactatacccaactcctaccccttcccctttactactactacacaaactataacttaacatctaaactaaaacatccaaaacacaatctccactcaaatcaccaacactccactcactccactaaaccacaaacccactaacactaaaatcccactaacactaaaatcccactaacactaaaatcccactaacactaaaatcccactaacactaaaatcccactaacactaaaatcccactaacactaaaatcccacaattatacacataaatccacataaccccatcccacacaattatacacataaatccacacaaccacatcccaccccactccaaaaaaccaacacaccaccacctaaaatattaaaactctcattatattctttaaactactttaaaaaatttcaacatacccctaaaatctaaaaattttaaaaattatttaaaaaaaaccaactctaaaaaaacctcacatccaatctcctatctcaacttcaaaccaaaattccaaattaaaatactcaaaaaaacaactaatccttctacccttacacaaaacaaaaatccccaaacctctttctaaaatacaaaatcttaatctttctcaaaaaatcaataaacctaaactcccaatctcttaaacccaaacataaataaaaaactaaaaaccaccaccatactcatatttctaataaccaaaaaatccaaacaaacttttacccactacaaacaaatctctttcttcaaaaatcaaaaaaaactctatacaataaaccaaccaaaccccaccaacaaaactacactacaaccaaattcctaatatctttcccacactcccaattccaaacaacacaaaacacaaccccaattccacctccccccacccacttcctaccctattacctcatcttccctactacacactccaaatcatccacccaacccccaactcctaataactcaaccccaactcctaaaaatccaaccccaaccctactaccaccacttccaccttaacctcccctaaaaacaaataaaaaacaaataaaacccaaacttaaattcactcatttaaaaaacaactcaaaccaaaccctataaaaacaatacctacctaaaacacctcaaactcatatacacctctaaattctttcaaacacctaccccaaatataaaaaaactactacccaatacctaaacccctctaactctctactcctctctaactatctctctatttttatcatctctcaccttcccctatcataaccaatacttaatcctcaaaccaaaaaaaataaatcccatatccctaaattttactaataccaaaaatcacataaatactcctatatatacacaaaaaaaacccacacatacttaaaaatcctcaaataccaaaaaacaaaatacaaaacacaacaaaaatactatacatattaaaaaatatatacatcccccaaatacaaaaaaatacacaccaacatacaaaaacacacctcacaaacacattcacaaaaacaaacacactcacactccaaaaaatactaaaacacccataaaaaaaacatttcaaacatacacaaaaccctaaactcacataaacacaaacacccaaaaacaaataaaccctaactcaaatacacacaacctaacacacaatactcacacacaaactcccctaaatctaaataccacaaaaaactcccacaaaaaaaaaattcccccaaaaacctccaaaaccctacctaaaaaaaaaaaaaaaaaaatacccaccaaacaccaaaatactaaaacactataaaataaaaatcatttccccaactccataaaaacaacacttccattcccatctcttaaaaaaacaaaaaaataatttaactcattctacaaatcaactccaaatccacaaaaataccctccccctcaacaaaaaaaaaccaaactcccaatactacaacccaataaatactaatccactaaaacccaccccaaaccacaaaatactaaacaaactcaccaaacccacacatctacctaacatctaactaaaccaaaaaaaatacctaataaacacccaacctaacaacacacaaaaaacacaaccaataaaaaaaataaatctatacaccaaaaacctacaccatctcaccaaactctaccctaacaaaccattccttacctttcttaatcacaaactaatctaaaaaactcattcccaactcatccattacctacaaataacaaaaaacaacaataaaaacacataaaaccctaaccacccatatcttcccaaacccctaccacccaaaatctctatctactccacccaaaacacaccaacttcacttaaaacaacaacaaacaaacccaaaaatcactatatccaaacactaaactaaaaccctaaacatctcactatacaatacaaaaaaaaaattttaaatcttacactaataactattatacttctcttcaccccaaatacaaactaccccataaataccacacataacaacaaaaaaaaaaaaaataattcaatccctaccaaaaattactaccacccacacactactaacaaactcaataccaaacaaccttaataaccaaacaacatctaacccaaaaaataaaacacatcctcaaaatcaccaataaaatataacaaactaaaaaaaaaatcaacttcctacatttatcaccttctacctccaaccataataataaaccaccacaaccaatcccaacccatcccaaatccctcaaatctcacctacaaatcctccaaaccaaaaaccaccaccaaccccaaaaaaccaaaaaatacatctaccaaaccatacacaccatcaaccaaaccatacaaaaaccaaaacacaacaataacataatcacaacacaaatcaaaacccaaaacacaaacaaacaataccaacaaaccaaaaaactcctcataaacacaaacaactacacaaaacacaacccaaacaacctaaaaaaactacaactacaccaaaaacaccaaaccaccatataaatcccctaccaaataaaaaaaaactacaacaacatcaaacacctaaccataaaacaacaataactacaaataaaacaacaaaaaataaaacaactaaaattcaacaacaactataacaaccaacatataacaaaacaaaaacaccaacccataaaccacctaaaacaaaaacaacaaacaaaccccaccaacaactactcccaacccatcaaaacactcctaaaaaaaaaaaaaaataccttaaataacccactaacaactaaaaaaaccccaacctaaaaaaatcctcctcaactcaaaaaaattcaactacaatccctcccatttcttaaaacccaaaatttatttccaaaccacccacaaacacaaaaacctcaaactaaaaacaacaatcacttacacatcacattctcaactaacaaaatccctacaaccccctccctccaaaatttcattaacacaaaacaaaaaaattaaaaaatccaacactcaaacaccccaatcccccaaccaaatatatcccaaacccaaaactactcaccataacaaaataaatatacaccaacataaaacaataaataccaaacacaaaacataacaacaacaaaaccctaacccaacaacaaacactacaaacaaaaacaccaaaaaccaacacaacccacaaaaatcacaatacaataactaaatacaacccaaccaaaatcctaataaacataaaatcctcaacaataaccattcccaaacaaatccataaaaacaaaacaaacaaacactaatcacacttaaaaaaatcaatcaacaaatacccaacacccacccttcctcctctaaacataacctactccatacactccaattataataacaaaaacacaccaaacctccaaaaacacacattaaaaaaacaaactataacaattctctcccttttccccaacaactaccacaaaaaaaacaataactttaacaaaatcaaataaaataaataaaaaacataaattcaacccacattaatcaaaaccaccttctaaactcacaactcacaactacaataaaacaaaaatattcaaaactaaaacacaaaaaccctacatttacctaactctatcacatccttacctttaaacaaaacactattcttcacta

SEQ ID NO: 6 - DNA, 1936 bp, Homo sapiens
taggcaaccacccactctcctccagagggaacgagggatccatctcagttctcatccttgaccactcccctccagctctgattgtataacctgaaacccacacacggtttgtaactttttttttttttttccgagacggagtctcgctctgtttccaggctggagtgcagtggcgcgatcttggctcactgcaatctccgcctcccgggttcaagcgattcccctggctcagcctccctagtagctgggactacagacgcgcaccaccatgcccggctaatttgtttgtaacctttttaacctgtctggcttttccacagggagaggatttgcatatgctgtgtcctgtgtgatgaaaggagccctttttacacaccccctctttcaatgcactcagacttcaaagcccatcactcattcattcaacaaacacccactaagcacctcttgaacccggttcaactatctcccctctaggcactcacccaacctcgggcactctccgcgcctcctctgagctcctcccgcgtcccatacagcccctgctcactgctggaaaatattcctgttaagctccgtctccccaccagtcctgcttgctttctgtgtgtctgggataggttgcaaaactggaggtgacaaatgtgggcaggaaatggagggctcccttatactcctagggtcggcctcagccctgccatttcctgcccaataccgcggatgcaactggcatgggacccggaagtgtgtggtaaagcgccggggcttcgcctggccgcccctcccggacgcttgcctgcagtcagcgaagcttttccaacccaggtctgggccctgcgagctccaggtgcctctgccccgcggcgctggcgaagtcgaagcccgagaacgcccaccgcagcgatgcgaaggccgcttctggggtggggctgaggctgcagctgcctccctttcgcaccaaggaccccaaccttcagcgacgcagccgccgctcccgcccaggctgggaggcactgtagggatccgacgcttcaggtggccaaagagcgactttctctgattccagggccctggcggggcaggctccagtaccgcactcggcggaggccgaaggtctgtggggcaggacaggagttcctcgtgccgtcggaagggccgaggacgaaggagggcgctaattcactccccactgggctggcggcaacgccgaatcccgcagtgaccgcggagggccaaggtgaaaactgctgggggcgccgagggcaggtgtggggaggggcggctccagggagcaaggagcccacctgcctcgccgccgcagctgccctgggtcgaccgcccacgctttcccctgggtcacgacctccggatccaactgtgcccggctcctgccccttcccctttgctgctgctgcgcgggctgtaacttgacgtctaggctggggcgtccagggcgcagtctccgctcaggtcgccagcgctccactcgctccactgggccacagacccgctggcgctggggtcccgctggcgctggggtcccgctggcgctggggtcccgctggcgctggggtcccgctggcgctggggtcccgctggcgctggggtcccgctggcgctggggtcccgcggttgtgcacgtgagtccgcgtggccccgtcccgcgcggttgtgcacgtgagtccgcgcggccgcgtcccgccccgctccagggagccagcgcgccgccacctgggatgttaggactctcgttgtgttctttggactgctttgggggatttcggcgtacccctaggatctaggagttttggaagttgtttgagagaaaccagctctgggagggcctcgcatccagtctcctgtctcggcttcggaccggggttccgggttaaggtgctcagaggaacagctgatccttctacccttgcgcagggca

SEQ ID NO: 7 - DNA, 1936 bp, Artificial Sequence
  Synthetic Construct TFAP2E Sense Bisulfite Sequence CpG island 1 (793 nt - 2728 nt) methylated
taggtaattatttatttttttttagagggaacgagggatttattttagtttttatttttgattatttttttttagttttgattgtataatttgaaatttatatacggtttgtaatttttttttttttttttcgagacggagtttcgttttgtttttaggttggagtgtagtggcgcgattttggtttattgtaattttcgtttttcgggtttaagcgatttttttggtttagtttttttagtagttgggattatagacgcgtattattatgttcggttaatttgtttgtaatttttttaatttgtttggtttttttatagggagaggatttgtatatgttgtgttttgtgtgatgaaaggagttttttttatatatttttttttttaatgtatttagattttaaagtttattatttatttatttaataaatatttattaagtattttttgaattcggtttaattattttttttttaggtatttatttaatttcgggtatttttcgcgttttttttgagtttttttcgcgttttatatagtttttgtttattgttggaaaatatttttgttaagtttcgtttttttattagttttgtttgttttttgtgtgtttgggataggttgtaaaattggaggtgataaatgtgggtaggaaatggagggtttttttatatttttagggtcggttttagttttgttattttttgtttaatatcgcggatgtaattggtatgggattcggaagtgtgtggtaaagcgtcggggtttcgtttggtcgtttttttcggacgtttgtttgtagttagcgaagtttttttaatttaggtttgggttttgcgagttttaggtgtttttgtttcgcggcgttggcgaagtcgaagttcgagaacgtttatcgtagcgatgcgaaggtcgtttttggggtggggttgaggttgtagttgtttttttttcgtattaaggattttaatttttagcgacgtagtcgtcgttttcgtttaggttgggaggtattgtagggattcgacgttttaggtggttaaagagcgattttttttgattttagggttttggcggggtaggttttagtatcgtattcggcggaggtcgaaggtttgtggggtaggataggagtttttcgtgtcgtcggaagggtcgaggacgaaggagggcgttaatttattttttattgggttggcggtaacgtcgaatttcgtagtgatcgcggagggttaaggtgaaaattgttgggggcgtcgagggtaggtgtggggaggggcggttttagggagtaaggagtttatttgtttcgtcgtcgtagttgttttgggtcgatcgtttacgtttttttttgggttacgattttcggatttaattgtgttcggtttttgtttttttttttttgttgttgttgcgcgggttgtaatttgacgtttaggttggggcgtttagggcgtagttttcgtttaggtcgttagcgttttattcgttttattgggttatagattcgttggcgttggggtttcgttggcgttggggtttcgttggcgttggggtttcgttggcgttggggtttcgttggcgttggggtttcgttggcgttggggtttcgttggcgttggggtttcgcggttgtgtacgtgagttcgcgtggtttcgtttcgcgcggttgtgtacgtgagttcgcgcggtcgcgtttcgtttcgttttagggagttagcgcgtcgttatttgggatgttaggattttcgttgtgttttttggattgttttgggggatttcggcgtatttttaggatttaggagttttggaagttgtttgagagaaattagttttgggagggtttcgtatttagttttttgtttcggtttcggatcggggtttcgggttaaggtgtttagaggaatagttgatttttttatttttgcgtagggta

SEQ ID NO: 8 - DNA, 1936 bp, Artificial Sequence
  Synthetic Construct TFAP2E Sense Bisulfite Sequence CpG island 1 (793 nt - 2728 nt) non-methylated
taggtaattatttatttttttttagagggaatgagggatttattttagtttttatttttgattatttttttttagttttgattgtataatttgaaatttatatatggtttgtaattttttttttttttttttgagatggagttttgttttgtttttaggttggagtgtagtggtgtgattttggtttattgtaatttttgttttttgggtttaagtgatttttttggtttagtttttttagtagttgggattatagatgtgtattattatgtttggttaatttgtttgtaatttttttaatttgtttggtttttttatagggagaggatttgtatatgttgtgttttgtgtgatgaaaggagttttttttatatatttttttttttaatgtatttagattttaaagtttattatttatttatttaataaatatttattaagtattttttgaatttggtttaattattttttttttaggtatttatttaattttgggtattttttgtgttttttttgagttttttttgtgttttatatagtttttgtttattgttggaaaatatttttgttaagttttgtttttttattagttttgtttgttttttgtgtgtttgggataggttgtaaaattggaggtgataaatgtgggtaggaaatggagggtttttttatatttttagggttggttttagttttgttattttttgtttaatattgtggatgtaattggtatgggatttggaagtgtgtggtaaagtgttggggttttgtttggttgttttttttggatgtttgtttgtagttagtgaagtttttttaatttaggtttgggttttgtgagttttaggtgtttttgttttgtggtgttggtgaagttgaagtttgagaatgtttattgtagtgatgtgaaggttgtttttggggtggggttgaggttgtagttgttttttttttgtattaaggattttaatttttagtgatgtagttgttgtttttgtttaggttgggaggtattgtagggatttgatgttttaggtggttaaagagtgattttttttgattttagggttttggtggggtaggttttagtattgtatttggtggaggttgaaggtttgtggggtaggataggagttttttgtgttgttggaagggttgaggatgaaggagggtgttaatttattttttattgggttggtggtaatgttgaattttgtagtgattgtggagggttaaggtgaaaattgttgggggtgttgagggtaggtgtggggaggggtggttttagggagtaaggagtttatttgttttgttgttgtagttgttttgggttgattgtttatgtttttttttgggttatgatttttggatttaattgtgtttggtttttgtttttttttttttgttgttgttgtgtgggttgtaatttgatgtttaggttggggtgtttagggtgtagtttttgtttaggttgttagtgttttatttgttttattgggttatagatttgttggtgttggggttttgttggtgttggggttttgttggtgttggggttttgttggtgttggggttttgttggtgttggggttttgttggtgttggggttttgttggtgttggggttttgtggttgtgtatgtgagtttgtgtggttttgttttgtgtggttgtgtatgtgagtttgtgtggttgtgttttgttttgttttagggagttagtgtgttgttatttgggatgttaggatttttgttgtgttttttggattgttttgggggattttggtgtatttttaggatttaggagttttggaagttgtttgagagaaattagttttgggagggttttgtatttagttttttgttttggttttggattggggttttgggttaaggtgtttagaggaatagttgatttttttatttttgtgtagggta

SEQ ID NO: 9 - DNA, 1936 bp, Artificial Sequence
  Synthetic Construct TFAP2E Antisense Bisulfite Sequence CpG island 1 (793 nt - 2728 nt) methylated
taaacaaccacccactctcctccaaaaaaaacgaaaaatccatctcaattctcatccttaaccactcccctccaactctaattatataacctaaaacccacacacgatttataactttttttttttttttccgaaacgaaatctcgctctatttccaaactaaaatacaataacgcgatcttaactcactacaatctccgcctcccgaattcaaacgattcccctaactcaacctccctaataactaaaactacaaacgcgcaccaccatacccgactaatttatttataacctttttaacctatctaacttttccacaaaaaaaaaatttacatatactatatcctatataataaaaaaaaccctttttacacaccccctctttcaatacactcaaacttcaaaacccatcactcattcattcaacaaacacccactaaacacctcttaaacccgattcaactatctcccctctaaacactcacccaacctcgaacactctccgcgcctcctctaaactcctcccgcgtcccatacaacccctactcactactaaaaaatattcctattaaactccgtctccccaccaatcctacttactttctatatatctaaaataaattacaaaactaaaaataacaaatataaacaaaaaataaaaaactcccttatactcctaaaatcgacctcaaccctaccatttcctacccaataccgcgaatacaactaacataaaacccgaaaatatataataaaacgccgaaacttcgcctaaccgcccctcccgaacgcttacctacaatcaacgaaacttttccaacccaaatctaaaccctacgaactccaaatacctctaccccgcgacgctaacgaaatcgaaacccgaaaacgcccaccgcaacgatacgaaaaccgcttctaaaataaaactaaaactacaactacctccctttcgcaccaaaaaccccaaccttcaacgacgcaaccgccgctcccgcccaaactaaaaaacactataaaaatccgacgcttcaaataaccaaaaaacgactttctctaattccaaaaccctaacgaaacaaactccaataccgcactcgacgaaaaccgaaaatctataaaacaaaacaaaaattcctcgtaccgtcgaaaaaaccgaaaacgaaaaaaaacgctaattcactccccactaaactaacgacaacgccgaatcccgcaataaccgcgaaaaaccaaaataaaaactactaaaaacgccgaaaacaaatataaaaaaaaacgactccaaaaaacaaaaaacccacctacctcgccgccgcaactaccctaaatcgaccgcccacgctttcccctaaatcacgacctccgaatccaactatacccgactcctaccccttcccctttactactactacgcgaactataacttaacgtctaaactaaaacgtccaaaacgcaatctccgctcaaatcgccaacgctccactcgctccactaaaccacaaacccgctaacgctaaaatcccgctaacgctaaaatcccgctaacgctaaaatcccgctaacgctaaaatcccgctaacgctaaaatcccgctaacgctaaaatcccgctaacgctaaaatcccgcgattatacacgtaaatccgcgtaaccccgtcccgcgcgattatacacgtaaatccgcgcgaccgcgtcccgccccgctccaaaaaaccaacgcgccgccacctaaaatattaaaactctcgttatattctttaaactactttaaaaaatttcgacgtacccctaaaatctaaaaattttaaaaattatttaaaaaaaaccaactctaaaaaaacctcgcatccaatctcctatctcgacttcgaaccgaaattccgaattaaaatactcaaaaaaacaactaatccttctacccttacgcaaaaca

SEQ ID NO: 10 - DNA, 1936 bp, Artificial Sequence
  Synthetic Construct TFAP2E Antisense Bisulfite Sequence CpG island 1 (793 nt - 2728 nt) non-methylated
taaacaaccacccactctcctccaaaaaaaacaaaaaatccatctcaattctcatccttaaccactcccctccaactctaattatataacctaaaacccacacacaatttataactttttttttttttttccaaaacaaaatctcactctatttccaaactaaaatacaataacacaatcttaactcactacaatctccacctcccaaattcaaacaattcccctaactcaacctccctaataactaaaactacaaacacacaccaccatacccaactaatttatttataacctttttaacctatctaacttttccacaaaaaaaaaatttacatatactatatcctatataataaaaaaaaccctttttacacaccccctctttcaatacactcaaacttcaaaacccatcactcattcattcaacaaacacccactaaacacctcttaaacccaattcaactatctcccctctaaacactcacccaacctcaaacactctccacacctcctctaaactcctcccacatcccatacaacccctactcactactaaaaaatattcctattaaactccatctccccaccaatcctacttactttctatatatctaaaataaattacaaaactaaaaataacaaatataaacaaaaaataaaaaactcccttatactcctaaaatcaacctcaaccctaccatttcctacccaataccacaaatacaactaacataaaacccaaaaatatataataaaacaccaaaacttcacctaaccacccctcccaaacacttacctacaatcaacaaaacttttccaacccaaatctaaaccctacaaactccaaatacctctaccccacaacactaacaaaatcaaaacccaaaaacacccaccacaacaatacaaaaaccacttctaaaataaaactaaaactacaactacctccctttcacaccaaaaaccccaaccttcaacaacacaaccaccactcccacccaaactaaaaaacactataaaaatccaacacttcaaataaccaaaaaacaactttctctaattccaaaaccctaacaaaacaaactccaataccacactcaacaaaaaccaaaaatctataaaacaaaacaaaaattcctcataccatcaaaaaaaccaaaaacaaaaaaaaacactaattcactccccactaaactaacaacaacaccaaatcccacaataaccacaaaaaaccaaaataaaaactactaaaaacaccaaaaacaaatataaaaaaaaacaactccaaaaaacaaaaaacccacctacctcaccaccacaactaccctaaatcaaccacccacactttcccctaaatcacaacctccaaatccaactatacccaactcctaccccttcccctttactactactacacaaactataacttaacatctaaactaaaacatccaaaacacaatctccactcaaatcaccaacactccactcactccactaaaccacaaacccactaacactaaaatcccactaacactaaaatcccactaacactaaaatcccactaacactaaaatcccactaacactaaaatcccactaacactaaaatcccactaacactaaaatcccacaattatacacataaatccacataaccccatcccacacaattatacacataaatccacacaaccacatcccaccccactccaaaaaaccaacacaccaccacctaaaatattaaaactctcattatattctttaaactactttaaaaaatttcaacatacccctaaaatctaaaaattttaaaaattatttaaaaaaaaccaactctaaaaaaacctcacatccaatctcctatctcaacttcaaaccaaaattccaaattaaaatactcaaaaaaacaactaatccttctacccttacacaaaaca

SEQ ID NO: 11 - DNA, 139 bp, Homo sapiens
ccggacgcttgcctgcagtcagcgaagcttttccaacccaggtctgggccctgcgagctccaggtgcctctgccccgcggcgctggcgaagtcgaagcccgagaacgcccaccgcagcgatgcgaaggccgcttctggg

SEQ ID NO: 12 - DNA, 139 bp, Artificial Sequence
  Synthetic Construct TFAP2E MSP amplificate region Bisulfite Sense (1577 nt - 1717 nt) methylated
tcggacgtttgtttgtagttagcgaagtttttttaatttaggtttgggttttgcgagttttaggtgtttttgtttcgcggcgttggcgaagtcgaagttcgagaacgtttatcgtagcgatgcgaaggtcgtttttggg

SEQ ID NO: 13 - DNA, 139 bp, Artificial Sequence
  Synthetic Construct TFAP2E MSP amplificate region Bisulfite Sense (1577 nt - 1717 nt) non-methylated
ttggatgtttgtttgtagttagtgaagtttttttaatttaggtttgggttttgtgagttttaggtgtttttgttttgtggtgttggtgaagttgaagtttgagaatgtttattgtagtgatgtgaaggttgtttttggg

SEQ ID NO: 14 - DNA, 139 bp, Artificial Sequence
  Synthetic Construct TFAP2E MSP amplificate region Bisulfite Antisense (1577 nt - 1717 nt) methylated
ccgaacgcttacctacaatcaacgaaacttttccaacccaaatctaaaccctacgaactccaaatacctctaccccgcgacgctaacgaaatcgaaacccgaaaacgcccaccgcaacgatacgaaaaccgcttctaaa

SEQ ID NO: 15 - DNA, 139 bp, Artificial Sequence
  Synthetic Construct TFAP2E MSP amplificate region Bisulfite Antisense (1577 nt - 1717 nt) non-methylated
ccaaacacttacctacaatcaacaaaacttttccaacccaaatctaaaccctacaaactccaaatacctctaccccacaacactaacaaaatcaaaacccaaaaacacccaccacaacaatacaaaaaccacttctaaa

SEQ ID NO: 16 - DNA, 5507 bp, Homo sapiens
gagtagctgggattacagacatctgccaccatgcccagctaattttttgtattttttagtagagacagggttttacagtgttggccagtctggtctcaaactcttgacctcgtgatccacccacctctgcctcccaaagtgctgggattacaggcgtgagccaccatgcctggcttcaattcttaaagatttgaattaattgagtccctagattagaaataatttttttggttacagagtcccatcagttttctggaaaaacggacaaaaggctaaagctagtttcattcatcacaaggtcgccatctcttagttctctagtttcaggctatttcacttgctacaagatttgattttaagagagatctgtatggaagcattttaaagttaatgtccaagattgagctcaaatgcaatgtggattcttctttattttgaaatatttatagcttttctattttttggcatactcttaatcgagcatgctgccgattgctggtcaattggcttcgacacagtagtccagggccacagtcgcaacgctggaagatttctggagcttgagcagtgtctttatgccctcttctggagcagacctgtccctccaaaaggactggcttacaaattttcgtccaaaaatgacgagcacagcaaagtccagggccacagtcaaaagttctcagacaactttctccctcttgtcctgtaacaaggttaatgtgggctttagtgataaataaggaaagtggcctgctgggggtttgctactgggataagggaatgggacctatgactgacagccacaaagtaaaaacagacacagcacgcaggtctcacagagctaggtgttacgctaaaagcagtaatgccatggcgactccacccctacccatcagaattgatcgttgttactggctgcagactaccttaggctactctctacgtgacccccacagcttcttacagaaagagataacaaacagcacatgcttcctaagtgtttctgggatggagaagaataaaatcagtctcaccctatccttaagaggctgggaggagaaaatgcatttctgaaataaaatatgctgcgaatgctgttcttacccttcctgccttgtgatttcttaatacttggcttccttttgggttggttttcctggactgggtgcccagttgttccttctgcatgtgtgccatcttgctcatcaagctgcctttctaatattggggttgcatcttccatcgtagtacaaacatctgagggacaaccaggtgtaactagaattattaacttcaagggggagaaccacagacacactaattatcctactgttgcatagtttggcagcacctctgccttcagtttatatataactttctagatttctggaacatgtgctaaatatttacatgctcgcatgtggcagagaaagtagctaagtggaaattgcattccattaaaggtagttaatggtatccaagtggaaatggaagtatttgaatgattacaacttgagtctttcctttaaaatcattatctccaagtcattggcaagagctgttgccaggaaataaattattttgtttcattaagaaaaggcaagccgggtccggtggcttacgcttgtaatcccagcactttgggaggccgaggtaggtggatcacctgaggtcagaagttcgagactagcctgaccaacatggtgaaaccctatctctactaaaaataacaaaaattagccgggcatggtggtacatgcctgtagtcctagttactccagaggctgaggcaggagaaccacctgaacccggaggccgaggctgcagtgagctgtggacaacaagggcgaaactccgcctcagaaaaaaaaaaaaaaggaaaagaaaaggcagctgcatacatttacttcaagcatgaatctaggaagcaccattagagagtgcctaccctggaatattctagtctatggggcttagacactaccttcggtttaaaaaccactgttggcgttatgtcgctcaccgttcacacagagtgtcccagggcagcacatggcatctcgctggcacctcctccgcaacccacgacatgtagcacagaacggcttctcatcgcggggctggaggcagaactttctggtattgcagtccgtgtcagacaggcactgtgagccctgtggagggaatctgttatcacaaggctaggaaacccccaacacgatggaagatgacttattattgaaggcaggaggcggagggagccaaaggacagtggcgctgtgacaaaccctgggcagcatcttactctccagtggaaatggttttctcccacaaagggaagctcatgtcacccggggagacctaggcgctgcaaaggctatttgagtttgaaaaattgacttaactcagaatttagttttcctgtgctctttgtgaaaatgggaaaaaatgttttcctatgtgtgtcttaacaggtgagtatgagattttcaatatatcttaactctagaagctggctacagtttcattaaacatactattgatgtttgtggcattcctatttgattttttactctagtgaatcacatttagcttagagacccttccctaagtgaactgtcctttgattttcaatgatgcctcattgacatagagaaagagataccgaagatcaattgttctcatacacagttgatgcttctacatagacatacaacgtaggtgtagcatatatgatatatgtaataatctatacgatatataataacatatacacatacataaagcgattcctaaaacaactctctctgtgaagttagatggtgacaaggcaatgcatcatggccctctgtttaaattctcattcccatgtttgcttgctggctgtgtgagcttgggtaagctcttcaacatctctgagtcttatttcctcatctggaattagggaatgtttcacctatctcaaattgtgaggaagagtacatgacgtattacatgtaaaagagctggcacatagtagagcttccaaagtgttcgtttatttcgtgatttatagacaaacgtatggccgaatgatgtgaaaagccactgtgtttatttcatacattcacactaaggccactgatcctaccaggatgccctcccaaatccgaaaccaattcaatttacctgctaggtaagacatttcatacatgaatcagaatgacttactaaagcctaaacaaaacaccccagctgtcagcagtcccgtacctcgcccccctccctagcagccttaccttccgggccccatgcaggtcagcagagctcctgatgttgttgaagtccaggaccagagctcccaggggagagcagagccagctcagccccagcaggacggccgccaccatccttcaatcccggggctcctggagggtcccagcactgtgcgtcaccaaagcgaggctgctctccacccagagcagagcttccactaagctggcagctcagcacgtcgtctgtttgtcactgctttttctgaaagaagtaaaccttagtctgagtaaggtgcgtgaaatcggctgagcaaagtctgaccagcaggttcctcctgaaactatttataaccagatgtgcctcctccccgctccgttccttcctcctgccccacacaggcttcaacaaatcccttcaaatctgtttgatcaatagttcaaaacaaggactcaatagaaggagagaagaggcagggccatgcggcttctcctccaggggttcaatccctcttaattattgcatatcaaagttagttcaaagggccacggcagagcaggatgtctgtatcaaaggcaaactctgataatcacctgcatttcctaccttcagtaatcctttgggggtagggagggagtctgccctaagatctgttttcacagctaattgtgctcaaagaaaggatgaatcatactggccaatatgctatagaggatgccaaaaactaatgaggtacaaaatacactgagaagctgtaaactgggcttcagatagtgaggacagatagaaaaagagaaaacgccccctgattagctctggcatccagatgtcaaaatcccaaccgcttaaaagttgtgaacgtaacagcacctgttctgactaaatgccatttgggaaattactttcctttaaatgaaaattgaactgtttgaagttagggttattaggaaaataatgtaatttatgaaaatcccatttttcaatggtgggaacaatgatagcgcattttacacaattcagtacgaagctttgggtccattttatcagcaacatatttaatttgtgttgcattatgtaaaactcacttaagcagaacaaggatccaagtctctggctgatttcagatgaaatttcatgtatttccaattttcctaatacgaattttttaaaacttctgcttcatccctttcctgtctgatttttttccatttggactattagtcaggtaatttagacatgtgcttatcactgtcttaaatactttaagggtagaacaatctagatctggatcaaaaaatcattttttctcttcattgtttcattgctcttagcatagagtcatctaaaatgcattttttctatgccataaatgataatcttattatttactacattttcctgcagaacaacctaacatcaattcattgacatgaatctcagcattctgtgcatgggaacattctcaattttgagtaatgcagtgccattccgagggtgatgcaggtgattcgagccttcctactacttggcccttgtccttgggttggtattgcttgtctacctctcgtatggaagttacttgttttaatcccttaaactgacatcaaacaacaccctttgatagcatgataaccctggtgactaggggtgttacagaatttattttcaagttgtacaattatgaccaaggataaaggaaagagcccccagggttctgtctctgccgggtttctctgcttcctgctgcctcactgtaagggaatttcaggttaccaaaggcccctggcaatatacacaggcgaaagaactgacgggaactcctgtaacacggcacaaacagccactgagcacttagagcacttggcatggaacagagcctcggattacatgttaggaataaaatttgaaactccaatggccgggcacagtggctcacacctgtaatcccagcactttgggaggccaaggtgggaagatctcctgagctcaggagttcaagacgagcctgggcaacatattgagatcctgtctccacaaaaaaaaaaaaaaaaaaaaaagtaggccgcgtgcggtggctgacgcctgtaatctcaccactttgggaggctgaggtggatggatcacctgaggtcaggagttcgagaccagcctggccaatatggtgaaaccctgtctctactaaaaatataaaaactagtcgggcgtggtggtggtcacctgtaatcccagctactcgggaggctgaggcaggagaa

SEQ ID NO: 17 - DNA, 20 bp, Artificial Sequence
  MSP_genomic_for
ccggacgcttgcctgcagtc

SEQ ID NO: 18 - DNA, 21 bp, Artificial Sequence
  Synthetic Construct MSP_genomic_rev
cccagaagcggccttcgcatc

SEQ ID NO: 19 - DNA, 22 bp, Artificial Sequence
  Synthetic Construct MSP_genomic_probe
agcccgagaacgcccaccgcag

SEQ ID NO: 20 - DNA, 20 bp, Artificial Sequence
  Synthetic Construct MSP_sense_bis_for
tcggacgtttgtttgtagtt

SEQ ID NO: 21 - DNA, 21 bp, Artificial Sequence
  Synthetic Construct MSP_sense_bis_rev
cccaaaaacgaccttcgcatc

SEQ ID NO: 22 - DNA, 22 bp, Artificial Sequence
  Synthetic Construct MSP_sense_bis_probe
agttcgagaacgtttatcgtag

SEQ ID NO: 23 - DNA, 20 bp, Artificial Sequence
  Synthetic Construct MSP_antisense_rev
ccgaacgcttacctacaatc

SEQ ID NO: 24 - DNA, 21 bp, Artificial Sequence
  Synthetic Construct MSP_antisense_for
tttagaagcggttttcgtatc

SEQ ID NO: 25 - DNA, 22 bp, Artificial Sequence
  Synthetic Construct MSP_antisense_probe
ttgcggtgggcgttttcgggtt

SEQ ID NO: 26 - DNA, 24 bp, Artificial Sequence
  Synthetic Construct BSP_sense_bis_for
tagtttgaaaggattttttttagt

SEQ ID NO: 27 - DNA, 26 bp, Artificial Sequence
  Synthetic Construct BSP_sense_bis_rev
actctaatcaatataaactaaattca

SEQ ID NO: 28 - DNA, 24 bp, Artificial Sequence
  Synthetic Construct Taenzer_BSP_antisense_bis_for
caacctaaaaaaatcctcctcaac

SEQ ID NO: 29 - DNA, 26 bp, Artificial Sequence
  Synthetic Construct BSP_antisense_bis_rev
gttttgattaatgtgggttgaattta

SEQ ID NO: 30 - DNA, 24 bp, Artificial Sequence
  Synthetic Construct BSP_genomic_for
cagcctgaaaggatcctcctcagc

SEQ ID NO: 31 - DNA, 26 bp, Artificial Sequence
  Synthetic Construct BSP_genomic_rev
gctctgatcaatgtgggctgaattca

SEQ ID NO: 32 - DNA, 24 bp, Artificial Sequence
  DKK Forward Primer 206bp
atattagaaaggcagcttgatgag

SEQ ID NO: 33 - DNA, 25 bp, Artificial Sequence
  DKK4 Reverse Primer bp 406-587
ttacaaattttcgtccaaaaatgac

SEQ ID NO: 34 - DNA, 24 bp, Artificial Sequence
  DKK4 Forward Primer 1kb
gaaagggatgaagcagaagtttta

SEQ ID NO: 35 - DNA, 23 bp, Artificial Sequence
  DKK4 Reverse Primer 2 put. AP2 BS
gtcgtctgtttgtcactgctttt

SEQ ID NO: 36 - DNA, 20 bp, Artificial Sequence
  DKK4 Forward Primer 2kb
ctcccaaagtgctgggatta

SEQ ID NO: 37 - DNA, 20 bp, Artificial Sequence
  DKK4 Reverse Primer 4 put. AP2 BS
gcacgtcgtctgtttgtcac

SEQ ID NO: 38 - DNA, 20 bp, Artificial Sequence
  DKK4 Forward Primer 500bp
tttaagcggttgggattttg

SEQ ID NO: 39 - DNA, 20 bp, Artificial Sequence
  DKK4 Reverse Primer 0 put. AP2 BS
taaccagatgtgcctcctcc

SEQ ID NO: 40 - DNA, 20 bp, Artificial Sequence
  DKK4 Forward Primer 114bp
ttcgcctgtgtatattgcca

SEQ ID NO: 41 - DNA, 20 bp, Artificial Sequence
  DKK4 Reverse Primer 2 put. AP2 BS ChIP 1
gataaaggaaagagccccca

SEQ ID NO: 42 - DNA, 20 bp, Artificial Sequence
  DKK4 Forward Primer 300bp
taagcggttgggattttgac

SEQ ID NO: 43 - DNA, 20 bp, Artificial Sequence
  DKK4 Reverse Primer 2 put. AP2 BS chip2
ggcagagcaggatgtctgta

SEQ ID NO: 44 - DNA, 25 bp, Artificial Sequence
  TFAP2E Forward Primer 310bp
tagaccagtccgtgatcaagaaagt

SEQ ID NO: 45 - DNA, 24 bp, Artificial Sequence
  TFAP2E Reverse Primer bp 747-1056
aggttgagcccaatcttctctaac

SEQ ID NO: 46 - DNA, 19 bp, Artificial Sequence
  TFAP2E Forward Primer 1331bp
cacctactccgccatggag

SEQ ID NO: 47 - DNA, 22 bp, Artificial Sequence
  TFAP2E Reverse Primer CDS
gtgggagaagcagttatttccg

SEQ ID NO: 48 - DNA, 25 bp, Artificial Sequence
  ACTB Forward Primer 132bp
tggtgatggaggaggtttagtaagt

SEQ ID NO: 49 - DNA, 27 bp, Artificial Sequence
  ACTB Reverse Primer
aaccaataaaacctactcctcccttaa

SEQ ID NO: 50 - DNA, 30 bp, Artificial Sequence
  Synthetic Construct ACTB Probe
accaccacccaacacacaataacaaacaca

SEQ ID NO: 51 - DNA, 22 bp, Artificial Sequence
  Synthetic Construct TFAP2A Forward
ctcgatccactccttacctcac

SEQ ID NO: 52 - DNA, 22 bp, Artificial Sequence
  Synthetic Construct TFAP2A Reverse
cctgcaggcagatttaatccta

SEQ ID NO: 53 - DNA, 22 bp, Artificial Sequence
  Synthetic Construct TFAP2B Reverse
aatggaagacgtccagtcagtt

SEQ ID NO: 54 - DNA, 22 bp, Artificial Sequence
  Synthetic Construct TFAP2B Reverse
agtgaacagcttctccttccac

SEQ ID NO: 55 - DNA, 22 bp, Artificial Sequence
  Synthetic Construct TFAP2C Forward
gatcagacagtcattcgcaaag

SEQ ID NO: 56 - DNA, 22 bp, Artificial Sequence
  Synthetic Construct TFAP2C Reverse
caaagtccctagccaaatgaac

SEQ ID NO: 57 - DNA, 22 bp, Artificial Sequence
  Synthetic Construct TFAP2D Forward
gccaaggtggagtgataagaag

SEQ ID NO: 58 - DNA, 22 bp, Artificial Sequence
  Synthetic Construct TFAP2D
ggcaagatgttctcctactgct

(19) United States
(12) Patent Application Publication
(10) Pub. No.: US 2011/0104695 A1
(43) Pub. Date: May 5, 2011

(54) METHODS OF PREDICTING THERAPEUTIC EFFICACY OF CANCER THERAPY

(75) Inventors: Catherine Lofton-Day, Seattle, WA (US); Reimo Tetzner, Berlin (DE); Matthias Philip Alexander Ebert, Munchen (DE); Marc Tänzer, Munchen (DE)

(73) Assignees: Epigenomics AG, Berlin (DE); Technische Universität München, Munchen (DE)

(21) Appl. No.: 12/939,949

(22) Filed: Nov. 4, 2010

(30) Foreign Application Priority Data
Nov. 5, 2009 (EP) 09175108.1
Mar. 24, 2010 (EP) 10157499.4
Aug. 19, 2010 (EP) 10173402.8

(51) Int. Cl. C12Q 1/68; G01N 33/68; G01N 33/53

(52) U.S. Cl. 435/6; 435/7.1; 436/86; 436/94

(57) ABSTRACT

The present invention relates to novel methods and kits for predicting, prognosing, and/or monitoring therapeutic efficacy of cancer therapy on a subject having malignant tumor or cell proliferative disorder.

### FIELD OF THE INVENTION

The present invention relates to novel methods and kits for predicting, prognosing, and/or monitoring therapeutic efficacy of cancer therapy on a subject having malignant tumour or cell proliferative disorder. In particular, the present invention relates to novel methods and kits for predicting, prognosing, and/or monitoring therapeutic efficacy of chemotherapy or chemoradiotherapy, in particular therapeutic efficacy of chemotherapeutic agents on a subject having malignant tumour or cell proliferative disorder. In addition, the present invention relates to use of the said methods and kits.

### BACKGROUND

Oncologists have a number of treatment options available to them, including different combinations of chemotherapeutic drugs. In recent years the treatment options for various cancers have profoundly improved through the introduction of polychemotherapies together with targeted antibody therapy. Since patients now undergo several consecutive lines of treatment, the molecular alterations underlying chemoresistance need to be better defined in order to individualize cancer therapy. Best likelihood of good treatment outcome requires that patients be assigned to optimal available cancer treatment. In particular, it is important to determine the likelihood of patient response to chemotherapy because chemotherapeutic drugs have limited efficacy and are toxic.

Progression free and overall survival has improved even in patients with distant metastasis. Since these therapies are now given in different treatment lines, identifying the molecular mechanisms of treatment response and/or resistance is becoming an important factor to better individualize cancer chemotherapy. So far knowledge on the molecular mechanisms of chemotherapy resistance in this and other cancers is very limited, despite the fact that the molecular biology of cancer initiation and progression has been elucidated in recent decades.

Most patients with advanced disease receive 5-FU based polychemotherapies, which in combination with novel targeted agents have demonstrated improved progression free and overall survival. The following publications which are incorporated by reference show the role of 5-FU in treatment of cell proliferative disorders, in particular colorectal, pancreatic, gastric, head and neck, breast, non-small-cell lung, leukaemia, and/or gynaecologic cancers: Comella P, Casaretti R, Sandomenico C, Avallone A, Franco L. Capecitabine, alone and in combination, in the management of patients with colorectal cancer: a review of the evidence. Drugs. 2008; 68(7):949-61. Review. PubMed PMID: 18457461; Geh J I, Bond S J, Bentzen S M, Glynne-Jones R. Systematic overview of preoperative (neoadjuvant) chemoradiotherapy trials in oesophageal cancer: evidence of a radiation and chemotherapy dose response. Radiother Oncol. 2006 March; 78(3):236-44. Epub 2006 Mar. 20. Review. PubMed PMID: 16545878; Gerszten K, Selvaraj R N, Kelley J, Faul C. Preoperative chemoradiation for locally advanced carcinoma of the vulva. Gynecol Oncol. 2005 December; 99(3):640-4. Epub 2005 Sep. 19. PubMed PMID: 16169579; Bourhis J, Calais G, Lapeyre M, Tortochaux J, Alfonsi M, Sire C, Bardet E, Rives M, Bergerot P, Rhein B, Desprez B; French Head and Neck Cancer Group (GORTEC). Concomitant radiochemotherapy or accelerated radiotherapy: analysis of two randomized trials of the French Head and Neck Cancer Group (GORTEC); Semin Oncol. 2004 December; 31(6):822-6. Review. PubMed PMID: 15599861; Aftimos P G, Nasr E A, Nasr D I, Noun R J, Nasr F L, Ghosn M G, El Helou J A, Chahine G Y. Adjuvant chemo-radiation for gastric adenocarcinoma: an institutional experience. Radiat Oncol. 2010 Jun. 4; 5(1):50. PubMed PMID: 20525367; PubMed Central PMCID: PMC2887890; Chua T C, Koh J L, Yan T D, Liam W, Morris D L. Cytoreductive surgery and perioperative intraperitoneal chemotherapy for peritoneal carcinomatosis from small bowel adenocarcinoma. J Surg Oncol. 2009 Aug. 1; 100(2):139-43. PubMed PMID: 19544356; Sultana A, Tudur Smith C, Cunningham D, Starling N, Neoptolemos J P, Ghaneh P; Meta-analyses of chemotherapy for locally advanced and metastatic pancreatic cancer: results of secondary end points analyses. Br J Cancer. 2008 Jul. 8; 99(1):6-13. Epub 2008 Jun. 24. PubMed PMID: 18577990; PubMed Central PMCID: PMC2453014; Hosono Y, Osada S, Nawa M, Takahashi T, Yamaguchi K, Kawaguchi Y, Yoshida K. Combination therapy of 5-fluorouracil with rapamycin for hormone receptor-negative human breast cancer. Anticancer Res. 2010 July; 30(7):2625-30. PubMed PMID: 20682991; and Auerbach M, Elias E G, Orford J. Experience with methotrexate, 5-fluorouracil, and leucovorin (MFL): a first line effective, minimally toxic regimen for metastatic breast cancer. Cancer Invest. 2002; 20(1):24-8. PubMed PMID: 11852998.

Fluoropyrimidines are the backbones of polychemotherapy in cancer therapy. Recently, drugs targeting the EGFR and VEGF have also been approved for the first-line treatment of patients with colorectal cancer. For instance, the overall survival of patients with colorectal cancer has increased from a median of 6-8 months in the era of 5-FU monotherapy to 24 months in patients receiving polychemotherapies with targeted drugs. Therefore, administration of these drugs in several lines of treatment is becoming a common strategy. Thus, individualization of cancer therapy is becoming routine and drugs need to be chosen on an individual basis with respect to certain clinical and genetic response predictors. Accordingly, recent studies have demonstrated that patients with mutant KRAS- or BRAF-cancer cells do not respond to EGFR inhibition. However, further molecular mechanisms underlying chemoresistance or allowing response prediction in this and other cancers are largely unknown.

5-FU is in the category of chemotherapeutic agents called antimetabolites. Antimetabolites attach specific phases of the cell cycle and most of them interfere with nucleic acid synthesis or nucleotide synthesis. 5-FU is a thymidylate synthase inhibitor and blocks synthesis of thymidine, leading to inhibition of DNA replication, inducing cell cycle arrest and apoptosis. 5-FU acts also as a pyrimidine analogue, inhibits replication enzymes and is transformed inside the cell into different cytotoxic metabolites. Other pyrimidine anatagonists are cytarabine (also called arabinosylcytosine or araC) which blocks DNA synthesis, decitabine which is a chemical analogue of cytidine which blocks DNA, RNA and protein synthesis, floxuridine which is a precursor to 5FU and which is metabolized in the body to 5FU, capecitabine which functions similar to floxuridine, Gemcitabine which is deoxycytidine analogue that has a broad spectrum of antitumour activity in many solid tumors. Resistance to 5-FU can be partly explained by mutations or polymorphisms in genes encoding drug catalyzing enzymes, like dihydropyrimidine dehydrogenase (DYPD) and methylenetetrahydrofolate reductase (MTHFR), leading to an increased risk of toxicity. Other genes have also been implicated in the mediation of 5-FU response including heat shock proteins and hypoxia induced factors, as well as cell adhesion molecules, and an apoptosis inhibitor. In addition, several methylated genes have been identified as potential markers for 5-FU resistance, such as MLH1 and MGMT.

Recently, epigenetic alterations underlying the pathogenesis of various cancers have been reported by several groups. These epigenetic alterations include hypo- and hypermethylation of the DNA, as well as modification of histones which all show a profound impact on transcriptional gene regulation. Further, aberrant methylation of CpG islands has been shown to lead to the transcriptional silencing of certain genes that have been previously linked to the pathogenesis of various cell proliferative disorders and cancers. CpG islands are sequences which are rich in CpG dinucleotides and can usually be found in the 5′ region of approximately 50% of all human genes. Methylation of the cytosines in these islands leads to the loss of gene expression and has been reported in the inactivation of the X chromosome and genomic imprinting. For example, multiple genes have been shown to be hypermethylated in the early steps of cancer, including APC, hMLH1 among others and increased expression of HDACs has been reported in this disease as well. However, the role of these molecular alterations in the context of response prediction and treatment resistance are far less well understood.

The AP2-transcription factor family consists of five members and plays important roles in both developmental (namely eye, skin and neural structures) and cancer biology. TFAP2A is a known tumour suppressor in various cancers and has been shown to be hypermethylated in renal cell carcinoma and B-Cell lymphoma. Similar results have been recently published for TFAP2C in breast cancer which acts as a tumour suppressor in this cancer. In prostate and colorectal cancer, TFAP2D and TFAP2E have been shown to be hypermethylated and were evaluated as potential methylation biomarkers in these diseases. The U.S. patent application Ser. No. 12/374,650 which is incorporated by reference, discloses association of hypermethylated TFAP2E with colorectal cancer. TFAP2E has 2 CpG islands, underscoring the potential for regulation of gene expression by CpG hypermethylation.

### DISCLOSURE OF THE INVENTION

### The Problem and its Solution

Although a variety of biomarkers have been identified, there still exists a need to identify a biomarker that can reliably predict, prognose, and/or monitor therapeutic efficacy of cancer therapy and its outcome on individual subjects having cell proliferative disorders, in particular on individual subjects having colorectal, pancreatic, gastric, head and neck, breast, non-small-cell lung, leukaemias, and/or gynaecologic cancers who have undergone or are undergoing chemotherapy, polychemothrapies, or chemoradiotherapy, preferably, 5-Flurouracil based chemotherapy or polychemothrapies.

Further, there still exists a need to identify a biomarker in order to select individual subjects having cell proliferative disorder or cancer for a particular cancer therapy and to identify individual subjects with cell proliferative disorder or cancer where a particular cancer therapy is unlikely to exhibit any clinical benefit.

Therefore, it would be highly desirable to be able to identify whether a subject with cell proliferative disorder or cancer who has undergone or is undergoing cancer therapy including chemotherapy, polychemotherapy, chemoradiotherapy, treatments with chemotherapeutic agents such as antimetabolites, in particular treatments with 5-Flurouracil based chemotherapy or polychemotherapies will be responsive to such treatments.

To solve the problems mentioned above, the present invention provides a method for predicting, prognosing, and/or monitoring therapeutic efficacy of cancer therapy on a subject having cell proliferative disorder by determining the methylation and/or expression levels of TFAP2E in a biological sample isolated from said subject. Further, the present invention identifies association of TFAP2E with treatment resistance towards chemotherapeutic agents, in particular 5-fluoruracil based chemotherapy or polychemotherapies, in vitro and in vivo, in various cell proliferative disorders and cancers, preferably colorectal, pancreatic, gastric, head and neck, breast, non-small-cell lung, leukaemia, and/or gynaecologic cancers.

### SUMMARY OF THE INVENTION

The present invention provides a method of predicting, prognosing, and/or monitoring, therapeutic efficacy of cancer therapy on a subject having malignant tumor or cell proliferative disorder comprising determining the methylation and/or expression levels of TFAP2E in a biological sample isolated from said subject, and predicting, prognosing, and/or monitoring therapeutic efficacy of cancer therapy on said subject based on methylation and/or expression levels of TFAP2E.

In further embodiment, the present invention provides a method of predicting, prognosing, and/or monitoring therapeutic efficacy of cancer therapy on a subject having malignant tumor or cell proliferative disorder by detecting the presence, absence or amount of CpG methylation within TFAP2E or sequence thereof.

In an embodiment, the present invention provides a method of predicting, prognosing, and/or monitoring therapeutic efficacy of cancer therapy on a subject having malignant tumor or cell proliferative disorder by detecting the presence, absence or level of mRNA transcribed from TFAP2E.

In further embodiment, the said method according to the present invention is performed detecting the presence, absence or level of a polypeptide encoded by TFAP2E or sequence thereof.

In an embodiment, the present invention provides a method of predicting, prognosing, and/or monitoring therapeutic efficacy of cancer therapy on a subject having malignant tumor or cell proliferative disorder wherein hypermethylation and/or decreased expression of TFAP2E is indicative of resistance to cancer therapy, while hypomethylation and/or increased expression of TFAP2E is indicative of sensitivity to cancer therapy.

In an embodiment, the cell proliferative disorder according to the present invention, preferably, is colorectal, pancreatic, gastric, head and neck, breast, non-small-cell lung, leukaemia, and/or gynaecologic cancers.

In another embodiment, the cancer therapy according to the present invention includes chemotherapy, polychemotherapy, or chemoradiotherapy treatments on a subject having malignant tumor or cell proliferative disorder, in particular includes treatments with chemotherapeutic agents, more in particular, treatments with antimetabolites, preferably pyrimidine antagonists including cytarabine, decitabine, floxuridine, capecitabine, and Gemcitabine, and more preferably, 5-Flurouracil based chemotherapy or polychemothrapies.

In further embodiment, the present invention provides a method of predicting, prognosing, and/or monitoring therapeutic efficacy of cancer therapy on a subject having malignant tumor or cell proliferative disorder by detecting the presence, absence or amount of CpG methylation within TFAP2E or sequence thereof comprising contacting genomic DNA isolated from a biological sample obtained from said subject with at least one reagent, or series of reagents that distinguishes between methylated and non-methylated CpG dinucleotides within at least one target region of the genomic DNA, wherein the target region comprises, or hybridizes under stringent conditions to a sequence of contiguous nucleotides of SEQ ID NO.: 1, wherein said contiguous nucleotides comprise at least one CpG dinucleotide sequence.

In addition to the above embodiment, the present invention provides a method of predicting, prognosing, and/or monitoring therapeutic efficacy of cancer therapy on a subject having malignant tumor or cell proliferative disorder by detecting the presence, absence or amount of CpG methylation within TFAP2E or sequence thereof, further comprising contacting the treated genomic DNA, or the treated fragment thereof, with an amplification enzyme and at least one oligonucleotide comprising, a contiguous sequence that is complementary to, or hybridizes under moderately stringent or stringent conditions to a sequence selected from a group consisting of SEQ ID NOs: 2-5, and complements thereof, wherein the treated genomic DNA or the fragment thereof is either amplified to produce at least one amplificate, or is not amplified; and determining, based on a presence, absence or amount of, or on a property of said amplificate, the methylation state or level of at least one CpG dinucleotide of SEQ ID NO: 1, or an average, or a value reflecting an average methylation state or level of a plurality of CpG dinucleotides of SEQ ID NO: 1, whereby predicting, prognosing, and/or monitoring of said subject is afforded.

In further embodiment, the present invention provides a method of predicting, prognosing, and/or monitoring therapeutic efficacy of cancer therapy on a subject having malignant tumor or cell proliferative disorder wherein treating the genomic DNA, or the fragment thereof, comprises use of a reagent selected from the group comprising of bisulfite, hydrogen sulfite, disulfite, and combinations thereof.

In further embodiment, the present invention provides a method of predicting, prognosing, and/or monitoring therapeutic efficacy of cancer therapy on a subject having malignant tumor or cell proliferative disorder wherein the biological sample obtained from the subject is selected from the group comprising metastatic cells, cell lines, histological slides, biopsies, paraffin-embedded tissue, frozen tissue, formalin fixed tissue, body fluids, stool, colonic effluent , urine, blood plasma, blood serum, whole blood, isolated blood cells, cells isolated from the blood and combinations thereof.

In further embodiment, the present invention provides a method of predicting, prognosing, and/or monitoring therapeutic efficacy of cancer therapy on a subject having malignant tumor or cell proliferative disorder comprising digesting the genomic DNA, or a fragment thereof, isolated from a biological sample obtained from said subject with one or more methylation sensitive restriction enzymes; contacting the DNA restriction enzyme digest of above with an amplification enzyme and at least one oligonucleotide suitable for the amplification of a sequence comprising at least one CpG dinucleotide of SEQ ID NO: 1; determining, based on a presence, absence or class of an amplificate the methylation state or level of at least one CpG dinucleotide of a sequence of SEQ ID NO: 1, and therefrom predicting, predicting, prognosing, and/or monitoring and/or monitoring of said subject is afforded.

In further embodiment, the present invention provides a kit for predicting, prognosing, and/or monitoring therapeutic efficacy of cancer therapy in a subject having malignant tumor or cell proliferative disorder who has undergone or is undergoing chemotherapy and/or chemoradiotherapy, characterized in that said kit comprises (a) one or more reagents to convert unmethylated cytosine bases to uracil or to anther base that is detectably dissimilar to cytosine in terms of hybridization properties (b) at least one oligonucleotide or polynucleotide able to hybridize under stringent or moderately stringent conditions to a sequence selected from a group consisting of SEQ ID NOs.: 2-5 in order to detect the presence, absence or amount of CpG methylation within TFAP2E, in particular a sequence SEQ ID NO.: 1, or sequence thereof c) containers suitable for containing the said one or more reagents and the biological sample of the patient; and optionally (d) instructions for use.

In further embodiment, the present invention provides a kit for predicting, prognosing, and/or monitoring therapeutic efficacy of cancer therapy in a subject having malignant tumor or cell proliferative disorder who has undergone or is undergoing chemotherapy and/or chemoradiotherapy, characterized in that said kit comprises (a) a means for detecting TFAP2E polypeptides; (b) a container suitable for containing the said means and the biological sample of the patient comprising the polypeptides wherein the means can form complexes with the polypeptides; (c) a means to detect the complexes of (b).

In further embodiment, the present invention provides a kit for predicting, prognosing, and/or monitoring therapeutic efficacy of cancer therapy in a subject having malignant tumor or cell proliferative disorder who has undergone or is undergoing chemotherapy and/or chemoradiotherapy, characterized in that said kit comprises (a) a methylation sensitive restriction enzyme reagent; (b) a container suitable for containing the said reagent and the biological sample of the patient; (c) at least one set of oligonucleotides one or a plurality of nucleic acids or peptide nucleic acids which are identical, are complementary, or hybridize under stringent or highly stringent conditions to a sequence of SEQ ID NO: 1; and optionally (d) instructions for use and interpretation of the kit results.

In further embodiment, the present invention relates to 5-azacytidine for treatment of a subject having malignant tumor or cell proliferative disorder, in particular colorectal cancer, wherein TFAP2E is hypermethylated.

Additional embodiments provide use of the methods and/or kits for predicting, prognosing, and/or monitoring therapeutic efficacy of cancer therapy on a subject having colorectal cancer.

### DRAWINGS

Those of skill in the art will understand that the drawings, described below, are for illustrative purposes only. The drawings are not intended to limit the scope of the present teachings in any way.

FIG. 1. Illustrates Luciferase reporter assays for (A) CACO-2 cells transfected with DKK4 promoter (1 kb and 2 kb fragments) and TFAP2E CDS, (B) SW480 cells transfected with DKK4 promoter (1 kb and 2 kb fragments) and TFAP2E CDS, (C) HT29 transfected with DKK4 promoter (1 kb fragment) and TFAP2E CDS, (D) Hek cells used as controls.* p<0.01.

FIG. 2. Illustrates chromatin immunoprecipitation. Relative quantification results for FLAG tagged stable TFAP2E overexpressing clones (FTS4, FTS10) with (A) putative AP2 protein binding sites 2 kb upstream in the promoter of the DKK4 gene and (B) putative AP2 protein binding sites 1 kb upstream in the promoter of the DKK4 gene.

FIG. 3. Illustrates drug resistance assays. Resistance to treatment with fluorouracil (5-FU), (A) SW480 cells transiently transfected with DKK4-CDS, TFAP2E-CDS and both (50.000 cells per well, fluorouracil 50 μg/ml daily on day 1-3); and (B) TFAP2E-CDS stable overexpressing clones and empty vector controls (4 clones per group, 50.000 cells per well; fluorouracil 50 μg/m daily on day 1-3); mean of 3 independent experiments performed in triplicates.

FIG. 4. Illustrates treatment responses in four patient cohorts and association with TFAP2E methylation. Response effects due to non-methylation with respect to the center affiliation of patients (A) as well as to the response evaluation technique (B).

FIG. 5. Shows sequence of the TFAP2E (A) and DKK4 (B) gene.

FIG. 6. Shows analysis of TFAP2E methylation in 74 colorectal cancer patients.

FIG. 7. Illustrates expression of TFAP2E and DKK4 mRNA in colorectal cancer cell, lines where n=untreated, a=5-azacytidine, T=Trichostatin A.

### DETAILED DESCRIPTION OF THE INVENTION

### Definitions

Unless defined otherwise, all technical and scientific terms used herein have the same meaning as is commonly understood by one of skill in art to which the subject matter herein belongs. As used herein, the following definitions are supplied in order to facilitate the understanding of the present invention.

The terms “therapeutic efficacy” or “ efficacy of cancer therapy” means to assess the reaction of a cancer to one or series of anti-cancer treatment(s) including but not limited to chemotherapy, chemoradiotherapy, polychemotherapy, chemotherapeutic agents, antimetabolites, pyrimidine antagonists, and 5-Flurouracil based chemotherapy or polychemothrapies. In other words, it refers to assessing the ability of a cancer to respond favourably or to resists cancer therapy.

The term “predicting” can comprise determining if a patient's tumour will respond to a particular type of therapy. A biomarker that is present prior to any therapy and which predicts that outcome.

The term “prognosing” can comprise determining the outcome of a patient's condition, the chance of recovery, or how the disease will progress. For example, a patient can obtain a prognosis of having a 50% chance of recovery based on results from obtaining a sample from the patient. It may also comprise providing information about the patient's likely course of disease in the absence of suitable therapy (how aggressive or non-aggressive a tumour is).

The term “Monitoring” can comprise reporting recurrence or progression of disease.

The term RECIST (Response Evaluation Criteria In Solid Tumors) refers to a set of published rules that define when cancer patients improve (“respond”), stay the same (“stable”) or worsen (“progression”) during treatments.

The known term DMEM (Dulbecco's modified Eagle's medium) refers to a cell culture medium that can be used to maintain cells in tissue culture.

“Transcription Element Search System” or TESS refers to a web tool for predicting transcription factor binding sites in DNA sequences. It can identify binding sites using site or consensus strings and positional weight matrices from the TRANSFAC, JASPAR, IMD, and our CBIL-GibbsMat database.

The abbreviation “CRC” used throughout the present application refers to colorectal cancer.

The term “Observed/Expected Ratio” (“O/E Ratio”) refers to the frequency of CpG dinucleotides within a particular DNA sequence, and corresponds to the [number of CpG sites/(number of C bases×number of G bases)]/band length for each fragment.

The term “CpG island” refers to a contiguous region of genomic DNA that satisfies the criteria of (1) having a frequency of CpG dinucleotides corresponding to an “Observed/Expected Ratio”>0.6, and (2) having a “GC Content”>0.5. CpG islands are typically, but not always, between about 0.2 to about 1 KB, or to about 2 kb in length.

The term “methylation state” or “methylation status” refers to the presence, absence or class of 5-methylcytosine (“5-mCyt”) at one or a plurality of CpG dinucleotides within a DNA sequence. Methylation states at one or more particular CpG methylation sites (each having two CpG dinucleotide sequences) within a DNA sequence include “unmethylated,” “fully-methylated” and “hemi-methylated.”

The term “hemi-methylation” or “hemimethylation” refers to the methylation state of a double stranded DNA wherein only one strand thereof is methylated.

The term ‘AUC’ as used herein is an abbreviation for the area under a curve. In particular it refers to the area under a Receiver Operating Characteristic (ROC) curve. The ROC curve is a plot of the true positive rate against the false positive rate for the different possible cut points of a diagnostic test. It shows the trade-off between sensitivity and specificity depending on the selected cut point (any increase in sensitivity will be accompanied by a decrease in specificity). The area under an ROC curve (AUC) is a measure for the accuracy of a test (the larger the area the better, optimum is 1, a random test would have a ROC curve lying on the diagonal with an area of 0.5; for reference: J. P. Egan. Signal Detection Theory and ROC Analysis, Academic Press, New York, 1975).

The term “microarray” refers broadly to both “DNA microarrays,” and ‘DNA chip(s),’ as recognized in the art, encompasses all art-recognized solid supports, and encompasses all methods for affixing nucleic acid molecules thereto or synthesis of nucleic acids thereon.

“Genetic parameters” are mutations and polymorphisms of genes and sequences further required for their regulation. To be designated as mutations are, in particular, insertions, deletions, point mutations, inversions and polymorphisms and, particularly preferred, SNPs (single nucleotide polymorphisms).

“Epigenetic parameters” are, in particular, cytosine methylation. Further epigenetic parameters include, for example, the acetylation of histones which, however, cannot be directly analysed using the described method but which, in turn, correlate with the DNA methylation.

The term “bisulfite reagent” refers to a reagent comprising bisulfite, disulfite, hydrogen sulfite or combinations thereof, useful as disclosed herein to distinguish between methylated and unmethylated CpG dinucleotide sequences.

The term “Methylation assay” refers to any assay for determining the methylation state of one or more CpG dinucleotide sequences within a sequence of DNA.

The term “MS.AP-PCR” (Methylation-Sensitive Arbitrarily-Primed Polymerase Chain Reaction) refers to the art-recognized technology that allows for a global scan of the genome using CG-rich primers to focus on the regions most likely to contain CpG dinucleotides, and described by Gonzalgo et al., Cancer Research 57:594-599, 1997.

The term “MethyLight™” refers to the art-recognized fluorescence-based real-time PCR technique described by Eads et al., Cancer Res. 59:2302-2306, 1999.

The term “Methylation assay” refers to any assay for determining the methylation state of one or more CpG dinucleotide sequences within a sequence of DNA.

The term “Methylation-Sensitive High Resolution Melting (MS-RHM) refers to a new approach for sensitive and high-throughput assessment of methylation. MS-HRM allows for estimation of the methylation level by comparing the melting profiles of unknown PCR products to the melting profiles of PCR products derived from standards with a known unmethylated to methylated template ratio.

The term “hybridisation” is to be understood as a bond of an oligonucleotide to a complementary sequence along the lines of the Watson-Crick base pairings in the sample DNA, forming a duplex structure.

The term “hypermethylation” refers to the average methylation state corresponding to an increased presence of 5-mCyt at one or a plurality of CpG dinucleotides within a DNA sequence of a test DNA sample, relative to the amount of 5-mCyt found at corresponding CpG dinucleotides within a normal control DNA sample.

The term “hypomethylation” refers to the average methylation state corresponding to a decreased presence of 5-mCyt at one or a plurality of CpG dinucleotides within a DNA sequence of a test DNA sample, relative to the amount of 5-mCyt found at corresponding CpG dinucleotides within a normal control DNA sample.

In particular preferred embodiments, the term “expression”, “expression level”, “over-expression”, “increased expression”, “increased expression level”, “under-expression”, “decreased expression level” or “decreased expression” refers to expression of mRNA, polypeptides or proteins as it is known in the art and which is based or influenced at least in parts by the presence or absence of methylation of a particular gene and/or its regulatory sequences. “over-expression”, “increased expression”, or “increased expression level” refers to expression which is based or influenced at least in parts by the absence of methylation. “under-expression”, “decreased expression level” or “decreased expression” refers to expression which is based or influenced at least in parts by the presence of methylation.

Primer extension: Several embodiments of the primer extension method exist. These methods are based on the conversion of unmethylated cytosines to uracil while methylated cytosines remain unchanged by means of bisulfite. The resulting converted DNA is then subjected to pre-amplification, in particular PCR. Thereafter a methylation specific elongation of a primer is carried out. The binding site of said primer is located up to 10 bases before the cytosine position to be analyzed, is adjacent to it, or encompasses it. After hybridization the primer is elongated by one up to 10 nucleotides. The said methods are for example described in U.S. Pat. No. 6,251,594, WO 01/62961, WO 01/062960, and WO 01/062064.

QM (Quantitative Methylation) method: According to this method, DNA is subjected to treatment, wherein methylated cytosine remains unchanged and unmethylated cytosine is converted to uracil or another base having different base pairing behaviour than cytosine. The converted DNA is then subjected to a real time PCR amplification in the presence of two probe systems. One probe system is specific for the case of methylation of the CpG positions of interest. The other probe system is specific for the case the CpG positions of interest are not methylated. Suitable probe systems are for example Taqman probes or LightCycler probes. In case of Taqman probes, each probes system consist of one probe. In case of Lightcycler probes, each probe system consists of two probes acting cooperatively. By means of the said probe systems, the amplification products derived from correspondent converted methylated or unmethylated DNA are detected during amplification. From this, the amount of original DNA being methylated or being unmethylated is deduced. The QM method is for example described in WO 2005/098035.

HQM (Heavy Quantitative Methylation) method: According to this method, unmethylated cytosines of a double stranded DNA are converted to uracil or a base having a different base pairing behaviour than cytosine while methylated cytosine remains unchanged. In preferred embodiments of the HQM method both converted strands are subjected to methylation analysis, in particular wherein originally reverse complementary regions are analysed in the subjected DNA. In particular preferred is the analysis of both converted DNA strands in methylation specific reactions. Such reactions may be: PCR, isothermal amplification, LCR, MSP, HeavyMethyl, bisulfite sequencing, microarrays, methylation specific restriction enzymes, real-time PCR, HeavyMethyl, MethyLight, QM, methylation sensitive primer extension. The HQM method is described in detail in WO 2008/017411

The term “MethyLight™” refers to the art-recognized fluorescence-based real-time PCR technique described by Eads et al., Cancer Res. 59:2302-2306, 1999. According to this method, DNA is treated with a modifying agent wherein unmethylated cytosines become modified. Thereafter the treated DNA is analyzed by real-time PCR with one or more probe. The binding site of said probe or probes comprises one or more CpG positions. Thus, the amount of amplification product derived either from DNA with methylated CpG positions or from DNA with unmethylated CpG positions is detected during amplification. From this, the amount of original DNA being methylated or unmethylated at the analysed CpG positions is deduced. Suitable probe systems for analysis are Taqman probe, LightCycler probes or Scorpion primers. The MethyLight method is for example described in WO 00/70090; U.S. Pat. No. 6,331,393; or Trinh et al.: DNA methylation analysis by MethyLight technology. Methods, 25:456-62, 2001).

The term “HeavyMethyl™” assay or “HM”, in the embodiment thereof implemented herein, refers to an assay, wherein methylation specific blocking probes (also referred to herein as blockers) covering CpG positions between, or covered by the amplification primers enable methylation-specific selective amplification of a nucleic acid sample. According to this method, in a first step, unmethylated cytosines of a DNA are converted to uracil while methylated cytosines remain unchanged. The converted DNA is then subjected to an amplification reaction. The amplification of the converted methylated or unmethylated DNA of interest is detected during the amplification while undesired background DNA is simultaneously suppressed by means of a blocker. Said blocker is either specific for background converted methylated DNA or background converted unmethylated DNA. Preferably, the HM method is carried out as a real time variant. The HM method is described for example in WO 02/072880.

The term “HeavyMethyl™ MethyLight™” assay, in the embodiment thereof implemented herein, refers to a HeavyMethyl™ MethyLight™ assay, which is a variation of the MethyLight™ assay, wherein the MethyLight™ assay is combined with methylation specific blocking probes covering CpG positions between the amplification primers.

The term “Ms-SNuPE” (Methylation-sensitive Single Nucleotide Primer Extension) refers to the art-recognized assay described by Gonzalgo & Jones, Nucleic Acids Res. 25:2529-2531, 1997.

The term “MSP” (Methylation-specific PCR) refers to the art-recognized methylation assay described by Herman et al. Proc. Natl. Acad. Sci. USA 93:9821-9826, 1996, and by U.S. Pat. No. 5,786,146. According to this method, DNA is treated with bisulfite resulting in a conversion of unmethylated cytosines to uracil while methylated cytosines remain unaltered. After said conversion, the DNA is subjected to PCR amplification by means of methylation-specific primers. The MSP method is for example described in Herman et al.: Methylation specific PCR: a novel PCR assay for methylation status of CpG islands. Proc Natl Acad Sci USA. 93:9821-6, 1996, When carried out as a real-time variant, the MSP allows a quantification of amplified products. Thus, it is possible to deduce the amount of subjected DNA being methylated or unmethylated at the cytosine positions of interest.

The term “COBRA” (Combined Bisulfite Restriction Analysis) refers to the art-recognized methylation assay described by Xiong & Laird, Nucleic Acids Res. 25:2532-2534, 1997. According to this method, DNA is treated with bisulfite. After the treatment, the DNA is amplified by means of PCR. Thereafter, the amplified DNA is digested by means of restriction enzymes. Said enzymes are specific for bisulfite treated unmethylated sites. Digested or undigested fragments are finally detected for example by gel electrophoresis and in if the case may be hybridization of a specific labeled probe. Methylation levels in the original DNA sample are represented by the relative amounts of digested and undigested PCR product. The COBRA method is for example described in Xiong & Laird, Nucleic Acids Res. 25:2532-2534, 1997.

MLA (Methylation Sensitive Ligation and Amplification): According to this method, DNA is subjected to a treatment wherein unmethylated cytosine is converted to uracil, while methylated cytosine remains unchanged. Preferably, said treatment is a treatment with bisulfite. Subsequently, the treated DNA is amplified by means of at least two pairs of essentially complementary probe oligonucleotides. The methylation status of the cytosine of interest in the original DNA is deduced from the presence or absence of amplificates. The MLA method is for example described in WO 03/057909.

The term “MCA” (Methylated CpG Island Amplification) refers to the methylation assay described by Toyota et al., Cancer Res. 59:2307-12, 1999, and in WO 00/26401A1.

The term “hybridization” is to be understood as a bond of an oligonucleotide to a complementary sequence along the lines of the Watson-Crick base pairings in the sample DNA, forming a duplex structure.

Direct sequencing: this sequencing method uses bisulfite-treated DNA utilized with PCR. Primers are designed to be strand-specific as well as bisulfite-specific. Both methylated and unmethylated sequences are amplified. All sites of unmethylated cytosines are portrayed as thymines in the resulting amplified sequence of the sense strand, and as adenines in the amplified antisense strand.

Pyrosequencing: Pyrosequencing is a real-time sequencing technology based on luminometric detection of pyrophosphate release upon nucleotide incorporation which is suited for simultaneous analysis and quantification of the methylation degree of several CpG positions. After bisulfite modification of genomic DNA, a region of interest is amplified by polymerase chain reaction (PCR) with one of the two primers being biotinylated. The PCR-generated template is rendered single stranded and a Pyrosequencing primer is annealed to analyze quantitatively CpG positions. After bisulfite treatment and PCR, the degree of each methylation at each CpG position in a sequence is determined from the ratio of T and C signals reflecting the proportion of unmethylated and methylated cytosines at each CpG site in the original sequence.

The terms “Methylation-specific restriction enzymes” or “methylation-sensitive restriction enzymes” shall be taken to mean an enzyme that selectively digests a nucleic acid dependant on the methylation state of its recognition site. In the case of such restriction enzymes which specifically cut if the recognition site is not methylated or hemimethylated, the cut will not take place, or with a significantly reduced efficiency, if the recognition site is methylated. In the case of such restriction enzymes which specifically cut if the recognition site is methylated, the cut will not take place, or with a significantly reduced efficiency if the recognition site is not methylated. Preferred are methylation-specific restriction enzymes, the recognition sequence of which contains a CG dinucleotide (for instance cgcg or cccggg). Further preferred for some embodiments are restriction enzymes that do not cut if the cytosine in this dinucleotide is methylated at the carbon atom C5. “Non-methylation-specific restriction enzymes” or “non-methylation-sensitive restriction enzymes” are restriction enzymes that cut a nucleic acid sequence irrespective of the methylation state with nearly identical efficiency. They are also called “methylation-unspecific restriction enzymes.”

“Stringent hybridization conditions,” as defined herein, involve hybridizing at 68° C. in 5×SSC/5× Denhardt's solution/1.0% SDS, and washing in 0.2×SSC/0.1% SDS at room temperature, or involve the art-recognized equivalent thereof (e.g., conditions in which a hybridization is carried out at 60° C. in 2.5×SSC buffer, followed by several washing steps at 37° C. in a low buffer concentration, and remains stable). Moderately stringent conditions, as defined herein, involve including washing in 3×SSC at 42° C., or the art-recognized equivalent thereof. The parameters of salt concentration and temperature can be varied to achieve the optimal level of identity between the probe and the target nucleic acid. Guidance regarding such conditions is available in the art, for example, by Sambrook et al., 1989, Molecular Cloning, A Laboratory Manual, Cold Spring Harbor Press, N.Y.; and Ausubel et al. (eds.), 1995, Current Protocols in Molecular Biology, (John Wiley & Sons, N.Y.) at Unit 2.10.

In reference to composite array sequences, the phrase “contiguous nucleotides” refers to a contiguous sequence region of any individual contiguous sequence of the composite array, but does not include a region of the composite array sequence that includes a “node,” as defined herein above.

The term “pre-cancerous” or “pre-neoplastic” and equivalents thereof shall be taken to mean any cellular proliferative disorder which is undergoing malignant transformation. Examples of such conditions include, in the context of colorectal cellular proliferative disorders, cellular proliferative disorders with a high degree of dysplasia and the following classes of adenomas:

Level 1: penetration of malignant glands through the muscularis mucosa into the submucosa, within the polyp head

Level 2: the same submucosal invasion, but present at the junction of the head to the stalk

Level 3: invasion of the stalk

Level 4: invasion of the stalk's base at the connection to the colonic wall (this level corresponds to stage Dukes A)

The term gene or genomic sequence shall be taken to include all transcript variants thereof and all promoter and regulatory elements thereof Furthermore as a plurality of SNPs are known within said gene the term shall be taken to include all sequence variants.

As used herein the term “prognosing” shall be taken to mean an indicator of the predicted progression of the disease (including but not limited to aggressiveness and metastatic potential) and/or predicted patient survival time.

In the context of the present invention the term ‘aggressiveness’ is taken to mean one or more of high likelihood of relapse post surgery; below average or below median patient survival; below average or below median disease free survival; below average or below median relapse-free survival; above average tumor-related complications; fast progression of tumor or metastases.

Unless stated otherwise as used herein the term “survival” shall be taken to include all of the following: survival until mortality, also known as overall survival (wherein said mortality may be either irrespective of cause or tumor related); “recurrence-free survival” (wherein the term recurrence shall include both localized and distant recurrence); metastasis free survival; disease free survival (wherein the term disease shall include cancer and diseases associated therewith). The length of said survival may be calculated by reference to a defined start point (e.g. time of diagnosis or start of treatment) and end point (e.g. death, recurrence or metastasis).

### Method of Present Invention

The present invention provides a method of predicting, prognosing, and/or monitoring therapeutic efficacy of cancer therapy on a subject having malignant tumor or cell proliferative disorder comprising determining the methylation and/or expression levels of TFAP2E in a biological sample isolated from said subject, and predicting, prognosing, and/or monitoring therapeutic efficacy of cancer therapy on said subject based on methylation and/or expression levels of TFAP2E. In one embodiment, the cancer therapy is chemotherapy, polychemotherapy, or chemoradiotherapy. In further embodiment, cancer therapy includes therapy with chemotherapeutic agents, in particular with antimetabolites. Cancer therapy also comprises treatments with pyrimidine antagonists including cytarabine, decitabine, floxuridine, capecitabine, and Gemcitabine. In further embodiment cancer therapy comprises 5-Flurouracil based chemotherapy or polychemothrapies.

The malignant tumor or cell proliferative disorder according to the present invention comprises colorectal, pancreatic, gastric, head and neck, breast, non-small-cell lung, leukaemia, and/or gynaecologic cancers.

The present invention provides for the use of the bisulfite technique, in combination with one or more methylation assays, for determination of the methylation status of CpG dinucleotide sequences within SEQ ID NO. 1, preferably SEQ ID NO. 6, and more preferably SEQ ID NO. 11. Genomic CpG dinucleotides can be methylated or unmethylated (alternatively known as up- and down-methylated respectively). Accordingly, when analyzing the methylation status of a CpG position within such a sample the person skilled in the art may use a quantitative assay for determining the level (e.g. percent, fraction, ratio, proportion or degree) of methylation at a particular CpG position as opposed to a methylation state. Accordingly the term methylation status or methylation state should also be taken to mean a value reflecting the degree of methylation at a CpG position. Unless specifically stated the terms “hypermethylated” or “upmethylated” shall be taken to mean a methylation level above that of a specified cut-off point, wherein said cut-off may be a value representing the average or median methylation level for a given population, or is preferably an optimized cut-off level. The “cut-off” is also referred herein as a “threshold”. In the context of the present invention the terms “methylated”, “hypermethylated” or “upmethylated” shall be taken to include a methylation level above the cut-off be zero (0)% (or equivalents thereof) methylation for all CpG positions within and associated with (e.g. in promoter or regulatory regions) of TFAP2E.

According to the present invention, determination of the methylation status of CpG dinucleotide sequences within SEQ ID NO. 1, preferably SEQ ID NO. 6, and more preferably SEQ ID NO. 11 has utility in the predicting, prognosing, and/or monitoring therapeutic efficacy of cancer therapy on a subject having malignant tumor or cell proliferative disorder.

### Methylation Assay Procedures.

Various methylation assay procedures are known in the art, and can be used in conjunction with the present invention. For example, the U.S. patent application Ser. No. 12/374,650 which is incorporated by reference, discloses methylation assay procedures of TFAP2E. These assays allow for determination of the methylation state of one or a plurality of CpG dinucleotides (e.g., CpG islands) within a DNA sequence. Such assays involve, among other techniques, DNA sequencing of bisulfite-treated DNA, RT-PCR or PCR (for sequence-specific amplification), Southern blot analysis. For example, genomic sequencing has been simplified for analysis of DNA methylation patterns and 5-methylcytosine distribution by using bisulfite treatment (Frommer et al., Proc. Natl. Acad. Sci. USA 89:1827-1831, 1992). Additionally, restriction enzyme digestion of PCR products amplified from bisulfite-converted DNA is used, e.g., the method described by Sadri & Hornsby (Nucl. Acids Res. 24:5058-5059, 1996), or COBRA (Combined Bisulfite Restriction Analysis) (Xiong & Laird, Nucleic Acids Res. 25:2532-2534, 1997).

It is particularly preferred that said reagent converts cytosine bases which are unmethylated at the 5′-position to uracil, thymine, or another base which is dissimilar to cytosine in terms of hybridization behaviour. However in an alternative embodiment said reagent may be a methylation sensitive restriction enzyme.

Wherein the genomic DNA sample is treated in such a manner that cytosine bases which are unmethylated at the 5′-position are converted to uracil, thymine, or another base which is dissimilar to cytosine in terms of hybridization behaviour It is preferred that this treatment is carried out with bisulfite (hydrogen sulfite, disulfite) and subsequent alkaline hydrolysis. Such a treatment results in the conversion of SEQ ID NO: 1, 6, and 11 to SEQ ID NO: 2-5, 7-10, and 12-15, respectively.

The treated genomic DNA is then analyzed in order to determine the methylation state of the target gene sequences. It is particularly preferred that the target region comprises, or hybridizes under stringent conditions to contiguous nucleotides of TFAP2E, preferably at least 16 contiguous nucleotides of TFAP2E. It is preferred that the sequence of said gene according to SEQ ID NO: 1 is analyzed, preferably SEQ ID NO. 6, and more preferably SEQ ID NO. 11. The method of analysis may be selected from those known in the art, including those listed herein. Particularly preferred are MS-RHM, MethyLight, MSP and the use of blocking oligonucleotides (HeavyMethyl) as described herein. It is further preferred that any oligonucleotides used in such analysis (including primers, blocking oligonucleotides and detection probes) should be reverse complementary, identical, or hybridize under stringent or highly stringent conditions to (preferably, an at least 9-base-pair long segment of) the base sequences of one or more of SEQ ID NO: 2-5, preferably SEQ ID NO.:7-10, and more preferably SEQ ID NO.:12-15 and sequences complementary thereto. Preferably,

The genomic sequences according to SEQ ID NO. 1, 6, 11 and non-naturally occurring treated variants thereof according to SEQ ID NO. 2-5, 7-10, 12-15, respectively were determined to have novel utility for predicting, prognosing, and/or monitoring therapeutic efficacy of cancer therapy on a subject having malignant tumor or cell proliferative disorder.

In one embodiment the method of the invention comprises the following steps: a) determining the methylation and/or expression levels of TFAP2E in a biological sample isolated from said subject; and predicting, prognosing, and/or monitoring therapeutic efficacy of cancer therapy on said subject based on step a). In a preferred embodiment said cancer therapy is chemotherapy, polychemotherapy or chemoradiotherapy. In further embodiment, cancer therapy includes therapy with chemotherapeutic agents, in particular with antimetabolites. Cancer therapy also comprises treatments with pyrimidine antagonists including cytarabine, decitabine, floxuridine, capecitabine, and Gemcitabine. In further embodiment cancer therapy comprises 5-Flurouracil based chemotherapy or polychemothrapies.

The malignant tumor or cell proliferative disorder according to the present invention comprises colorectal, pancreatic, gastric, head and neck, breast, non-small-cell lung, leukaemia, and/or gynaecologic cancers.

In further embodiment of the present invention, predicting, prognosing, and/or monitoring therapeutic efficacy of cancer therapy on a subject having malignant tumor or cell proliferative disorder comprises a) determining the methylation and/or expression levels of TFAP2E in a biological sample isolated from said subject; b) predicting, prognosing, and/or monitoring therapeutic efficacy of cancer therapy on said subject based on step a). Preferably thereby, hypermethylation and/or decreased expression of TFAP2E is indicative of resistance to cancer therapy, while hypomethylation and/or increased expression of TFAP2E is indicative of sensitivity to cancer therapy.

The method of the invention may be enabled by means of any analysis of the expression of an RNA transcribed therefrom or polypeptide or protein translated from said RNA, preferably by means of mRNA expression analysis or polypeptide expression analysis.

Accordingly the present invention also provides prediction, prognostic, and/or monitoring assays and methods, both quantitative and qualitative for detecting the expression of TFAP2E in a subject and determining therefrom the predicting, prognosing, and/or monitoring therapeutic efficacy of cancer therapy on a subject having malignant tumor or cell proliferative disorder.

In further embodiment, the present invention provides a kit for predicting, prognosing, and/or monitoring therapeutic efficacy of cancer therapy in a subject having malignant tumor or cell proliferative disorder who has undergone or is undergoing chemotherapy and/or chemoradiotherapy, characterized in that said kit comprises (a) one or more reagents to convert unmethylated cytosine bases to uracil or to anther base that is detectably dissimilar to cytosine in terms of hybridization properties (b) at least one oligonucleotide or polynucleotide able to hybridize under stringent or moderately stringent conditions to a sequence selected from a group consisting of SEQ ID NOs.: 2-5, preferably SEQ ID NOs.: 7-10, and more preferably SEQ ID NOs.: 12-15 in order to detect the presence, absence or amount of CpG methylation within TFAP2E, in particular a sequence SEQ ID NO.: 1, preferably SEQ ID NO.: 6, more preferably SEQ ID NO.:11. Respectively, or sequence thereof c) containers suitable for containing the said one or more reagents and the biological sample of the patient; and optionally (d) instructions for use.

In further embodiment, the present invention provides a kit for predicting, prognosing, and/or monitoring therapeutic efficacy of cancer therapy in a subject having malignant tumor or cell proliferative disorder who has undergone or is undergoing chemotherapy and/or chemoradiotherapy, characterized in that said kit comprises (a) a means for detecting TFAP2E polypeptides; (b) a container suitable for containing the said means and the biological sample of the patient comprising the polypeptides wherein the means can form complexes with the polypeptides; (c) a means to detect the complexes of (b).

In further embodiment, the present invention provides a kit for predicting, prognosing, and/or monitoring therapeutic efficacy of cancer therapy in a subject having malignant tumor or cell proliferative disorder who has undergone or is undergoing chemotherapy and/or chemoradiotherapy, characterized in that said kit comprises (a) a methylation sensitive restriction enzyme reagent; (b) a container suitable for containing the said reagent and the biological sample of the patient; (c) at least one set of oligonucleotides one or a plurality of nucleic acids or peptide nucleic acids which are identical, are complementary, or hybridize under stringent or moderately stringent conditions to a sequence of SEQ ID NO: 1, preferably SEQ ID NO.: 6, more preferably SEQ ID NO.:11; and optionally (d) instructions for use and interpretation of the kit results.

In a further embodiment of the present invention the said kit further comprising at least one oligonucleotide which is identical, is complementary, or hybridizes under stringent or moderately stringent conditions to at least 9 base long segment of a sequence selected from SEQ ID NOs.: 1, 2-5, preferably SEQ ID NO.: 6, 7-10, more preferably SEQ ID NO.:11, 12-15.

The kit may also contain other components such as hybridisation buffer (where the oligonucleotides are to be used as a probe) packaged in a separate container. Alternatively, where the oligonucleotides are to be used to amplify a target region, the kit may contain, packaged in separate containers, a polymerase and a reaction buffer optimised for primer extension mediated by the polymerase, such as PCR. Preferably said polymerase is a reverse transcriptase. It is further preferred that said kit further contains an Rnase reagent.

The present invention further provides for methods for the detection of the presence of the polypeptide encoded by TFAP2E sequences in a sample obtained from said subject.

Aberrant levels of polypeptide expression of the polypeptides encoded by TFAP2E are associated with the prediction, predicting, prognosing, and/or monitoring, and or monitoring of a subject having malignant tumor or cell proliferative disorder.

According to the present invention under-expression of said polypeptides is associated with a negative prediction and/or predicting, prognosing, and/or monitoring therapeutic efficacy of cancer therapy on a subject having malignant tumor or cell proliferative disorder. Any method known in the art for detecting polypeptides can be used. Such methods include, but are not limited to mass-spectrometry, immunodiffusion, immunoelectrophoresis, immunochemical methods, binder-ligand assays, immunohistochemical techniques, agglutination and complement assays (e.g., see Basic and Clinical Immunology, Sites and Ten, eds., Appleton & Lange, Norwalk, Conn. pp 217-262, 1991 which is incorporated by reference). Preferred are binder-ligand immunoassay methods including reacting antibodies with an epitope or epitopes and competitively displacing a labelled polypeptide or derivative thereof.

Certain embodiments of the present invention comprise the use of antibodies specific to the polypeptide(s) encoded by TFAP2E. Such antibodies are useful for predicting, prognosing, and/or monitoring therapeutic efficacy of cancer therapy on a subject having malignant tumor or cell proliferative disorder. In certain embodiments production of monoclonal or polyclonal antibodies can be induced by the use of an epitope encoded by a polypeptide of TFAP2E as an antigene. Such antibodies may in turn be used to detect expressed polypeptides. The levels of such polypeptides present may be quantified by conventional methods. Antibody-polypeptide binding may be detected and quantified by a variety of means known in the art, such as labelling with fluorescent or radioactive ligands. The invention further comprises kits for performing the above-mentioned procedures, wherein such kits contain antibodies specific for the investigated polypeptides.

Numerous competitive and non-competitive polypeptide binding immunoassays are well known in the art. Antibodies employed in such assays may be unlabelled, for example as used in agglutination tests, or labelled for use a wide variety of assay methods. Labels that can be used include radionuclides, enzymes, fluorescers, chemiluminescers, enzyme substrates or co-factors, enzyme inhibitors, particles, dyes and the like. Preferred assays include but are not limited to radioimmunoassay (RIA), enzyme immunoassays, e.g., enzyme-linked immunosorbent assay (ELISA), fluorescent immunoassays and the like. Polyclonal or monoclonal antibodies or epitopes thereof can be made for use in immunoassays by any of a number of methods known in the art.

In an alternative embodiment of the method the proteins may be detected by means of western blot analysis. Said analysis is standard in the art, briefly proteins are separated by means of electrophoresis e.g. SDS-PAGE. The separated proteins are then transferred to a suitable membrane (or paper) e.g. nitrocellulose, retaining the spacial separation achieved by electrophoresis. The membrane is then incubated with a blocking agent to bind remaining sticky places on the membrane, commonly used agents include generic protein (e.g. milk protein). An antibody specific to the protein of interest is then added, said antibody being detectably labelled for example by dyes or enzymatic means (e.g. alkaline phosphatase or horseradish peroxidase). The location of the antibody on the membrane is then detected.

In an alternative embodiment of the method the proteins may be detected by means of immunohistochemistry (the use of antibodies to probe specific antigens in a sample). Said analysis is standard in the art, wherein detection of antigens in tissues is known as immunohistochemistry, while detection in cultured cells is generally termed immunocytochemistry. Briefly the primary antibody to be detected by binding to its specific antigen. The antibody-antigen complex is then bound by a secondary enzyme conjugated antibody. In the presence of the necessary substrate and chromogen the bound enzyme is detected according to coloured deposits at the antibody-antigen binding sites. There is a wide range of suitable sample types, antigen-antibody affinity, antibody types, and detection enhancement methods. Thus optimal conditions for immunohistochemical or immunocytochemical detection must be determined by the person skilled in the art for each individual case.

One approach for preparing antibodies to a polypeptide is the selection and preparation of an amino acid sequence of all or part of the polypeptide, chemically synthesising the amino acid sequence and injecting it into an appropriate animal, usually a rabbit or a mouse (Milstein and Kohler Nature 256:495-497, 1975; Gulfre and Milstein, Methods in Enzymology: Immunochemical Techniques 73:1-46, Langone and Banatis eds., Academic Press, 1981 which are incorporated by reference in its entirety). Methods for preparation of the polypeptides or epitopes thereof include, but are not limited to chemical synthesis, recombinant DNA techniques or isolation from biological samples.

In the final step of the method the predicting, prognosing, and/or monitoring of the subject is determined, whereby under-expression (of mRNA or polypeptides) is indicative of non-responsivenss to chemotherapy, in particular 5-FU based chemotherapy on a subject having malignant tumor or cell proliferative disorder. The term under-expression shall be taken to mean expression at a detected level less than a pre-determined cut off which may be selected from the group consisting of the mean, median or an optimised threshold value. The term over-expression shall be taken to mean expression at a detected level greater than a pre-determined cut off which may be selected from the group consisting of the mean, median or an optimised threshold value.

Another aspect of the invention provides a kit for use in determining the predicting, prognosing, and/or monitoring therapeutic efficacy of cancer therapy on a subject having malignant tumor or cell proliferative disorder according to the methods of the present invention, comprising: a means for detecting TFAP2E polypeptides. The means for detecting the polypeptides comprise preferably antibodies, antibody derivatives, or antibody fragments. The polypeptides are most preferably detected by means of Western Blotting utilizing a labelled antibody. In another embodiment of the invention the kit further comprising means for obtaining a biological sample of the subject.

### Methylation Analysis

Particular embodiments of the present invention provide a novel application of the analysis of methylation levels and/or patterns within TFAP2E that enables prediction, prognosing, and/or monitoring of a subject having malignant tumor or cell proliferative disorder. Prediction, prognosing, and/or monitoring of a patient having having malignant tumor or cell proliferative disorder enables the physician to make better and more informed treatment decisions. In one embodiment said predicting, prognosing, and/or monitoring is on a subject with having malignant tumor or cell proliferative disorder who has undergone or is undergoing cancer therapy, In a preferred embodiment said cancer therapy is chemotherapy, polychemotherapy or chemoradiotherapy. In further embodiment, cancer therapy includes therapy with chemotherapeutic agents, in particular with antimetabolites. Cancer therapy also comprises treatments with pyrimidine antagonists including cytarabine, decitabine, floxuridine, capecitabine, and Gemcitabine. In further embodiment cancer therapy comprises 5-Flurouracil based chemotherapy or polychemothrapies.

In the preferred embodiment of the method, the predicting, prognosing, and/or monitoring therapeutic efficacy of cancer therapy on a subject having malignant tumor or cell proliferative disorder is determined by analysis of the methylation status of one or more CpG dinucleotides of TFAP2E gene.

In one embodiment the invention of said method comprises the following steps: i) contacting genomic DNA (preferably isolated from metastatic cells, cell lines, histological slides, biopsies, paraffin-embedded tissue, frozen tissue, formalin fixed tissue, body fluids, stool, colonic effluent , urine, blood plasma, blood serum, whole blood, isolated blood cells, cells isolated from the blood and combinations thereof) obtained from the subject with at least one reagent, or series of reagents that distinguishes between methylated and non-methylated CpG dinucleotides within TFAP2E (including promoter and regulatory regions thereof) and ii) predicting, prognosing, and/or monitoring of said subject having malignant tumor or cell proliferative disorder based on results of step i).

It is preferred that said one or more CpG dinucleotides of TFAP2E are comprised within a respective genomic target sequence thereof as provided in SEQ ID NO. 1 and complements thereof, preferably SEQ ID NO. 6, and more preferably SEQ ID NO. 11 and their complements therof. The present invention further provides a method for ascertaining genetic and/or epigenetic parameters of TFAP2E and/or the genomic sequence according to SEQ ID NO. 1 preferably SEQ ID NO. 6, and more preferably SEQ ID NO. 11 within a subject by analyzing cytosine methylation. Said method comprises contacting a nucleic acid comprising SEQ ID NO. 1, preferably SEQ ID NO. 6, and more preferably SEQ ID NO. 11 in a biological sample obtained from said subject with at least one reagent or a series of reagents, wherein said reagent or series of reagents, distinguishes between methylated and non-methylated CpG dinucleotides within the target nucleic acid.

In a preferred embodiment, said method comprises the following steps: In the first step, a sample of the tissue to be analyzed is obtained. The source may be any suitable source, such as metastatic cells, cell lines, histological slides, biopsies, paraffin-embedded tissue, frozen tissue, formalin fixed tissue, body fluids, stool, colonic effluent, urine, blood plasma, blood serum, whole blood, isolated blood cells, cells isolated from the blood and combinations thereof.

The genomic DNA is then isolated from the sample. Genomic DNA may be isolated by any means standard in the art, including the use of commercially available kits. Briefly, wherein the DNA of interest is encapsulated in by a cellular membrane the biological sample must be disrupted and lysed by enzymatic, chemical or mechanical means. The DNA solution may then be cleared of proteins and other contaminants e.g. by digestion with proteinase K. The genomic DNA is then recovered from the solution. This may be carried out by means of a variety of methods including salting out, organic extraction or binding of the DNA to a solid phase support. The choice of method will be affected by several factors including time, expense and required quantity of DNA.

Wherein the sample DNA is not enclosed in a membrane (e.g. circulating DNA from a blood sample) methods standard in the art for the isolation and/or purification of DNA may be employed. Such methods include the use of a protein degenerating reagent e.g. chaotropic salt e.g. guanidine hydrochloride or urea; or a detergent e.g. sodium dodecyl sulphate (SDS), cyanogen bromide. Alternative methods include but are not limited to ethanol precipitation or propanol precipitation, vacuum concentration amongst others by means of a centrifuge. The person skilled in the art may also make use of devices such as filter devices e.g. ultrafiltration, silica surfaces or membranes, magnetic particles, polystyrol particles, polystyrol surfaces, positively charged surfaces, and positively charged membranes, charged membranes, charged surfaces, charged switch membranes, charged switched surfaces.

Once the nucleic acids have been extracted, the genomic double stranded DNA is used in the analysis, methylation analysis may be carried out by any means known in the art including but not limited to methylation sensitive restriction enzyme analysis and chemical reagent analysis.

### Chemical Analysis

In the second step of the method, the genomic DNA sample is treated in such a manner that cytosine bases which are unmethylated at the 5′-position are converted to uracil, thymine, or another base which is dissimilar to cytosine in terms of hybridisation behaviour, This will be understood as ‘pre-treatment’ or ‘treatment’ herein.

This is preferably achieved by means of treatment with a bisulfite reagent. The term “bisulfite reagent” refers to a reagent comprising bisulfite, disulfite, hydrogen sulfite or combinations thereof, useful as disclosed herein to distinguish between methylated and unmethylated CpG dinucleotide sequences. Methods of said treatment are known in the art (e.g. PCT/EP2004/011715, which is incorporated by reference in its entirety). It is preferred that the bisulfite treatment is conducted in the presence of denaturing solvents such as but not limited to n-alkylenglycol, particularly diethylene glycol dimethyl ether (DME), or in the presence of dioxane or dioxane derivatives. In a preferred embodiment the denaturing solvents are used in concentrations between 1% and 35% (v/v). It is also preferred that the bisulfite reaction is carried out in the presence of scavengers such as but not limited to chromane derivatives, e.g., 6-hydroxy-2,5,7,8,-tetramethylchromane 2-carboxylic acid or trihydroxybenzoe acid and derivates thereof, e.g. Gallic acid (see: PCT/EP2004/011715 which is incorporated by reference in its entirety). The bisulfite conversion is preferably carried out at a reaction temperature between 30° C. and 70° C., whereby the temperature is increased to over 85° C. for short periods of times during the reaction (see: PCT/EP2004/011715 which is incorporated by reference in its entirety). The bisulfite treated DNA is preferably purified priori to the quantification. This may be conducted by any means known in the art, such as but not limited to ultrafiltration, preferably carried out by means of Microcon\textcircled{}™ columns (manufactured by Millipore\textcircled{}™). The purification is carried out according to a modified manufacturer's protocol (see: PCT/EP2004/011715 which is incorporated by reference in its entirety).

In the third step of the method, fragments of the treated DNA are amplified, using sets of primer oligonucleotides according to the present invention, and an amplification enzyme. The amplification of several DNA segments can be carried out simultaneously in one and the same reaction vessel. Typically, the amplification is carried out using a polymerase chain reaction (PCR) or Real Time polymerase chain reaction (RT-PCR). Preferably said amplificates are 100 to 2,000 base pairs in length. The primer oligonucleotides includes at least one oligonucleotides whose sequence is reverse complementary, identical, or hybridize under stringent, moderately or highly stringent conditions to the base sequences of one of SEQ ID NOs. 2-5, preferably SEQ ID NOs. 7-10, and more preferably SEQ ID NO.s: 12-15 and sequences complementary thereto, preferably to an at least 9, 16, or 20-base-pair long segment of the base sequences of one of SEQ ID NOs. 2-5 preferably SEQ ID NOs. 7-10, and more preferably SEQ ID NO.s: 12-15.

In an alternate embodiment of the method, the methylation status of pre-selected CpG positions within TFAP2E and preferably within the nucleic acid sequences according to SEQ ID NO.1 preferably SEQ ID NO 6, and more preferably SEQ ID NO. 11 may be detected by use of methylation-specific primer oligonucleotides. This technique (MSP) has been described in U.S. Pat. No. 6,265,171 to Herman. The use of methylation status specific primers for the amplification of bisulfite treated DNA allows the differentiation between methylated and unmethylated nucleic acids. MSP primers pairs contain at least one primer which hybridizes to a bisulfite treated CpG dinucleotide. Therefore, the sequence of said primers comprises at least one CpG dinucleotide. MSP primers specific for non-methylated DNA contain a “T’ at the position of the C position in the CpG. Preferably, therefore, the base sequence of said primers comprise a sequence having a length of at least 9, 16, or 20 nucleotides which hybridizes to a treated nucleic acid sequence according to one of SEQ ID NOs. 2-5 preferably SEQ ID NOs. 7-10, and more preferably SEQ ID NO.s: 12-15 and sequences complementary thereto, wherein the base sequence of said oligomers comprises at least one CpG dinucleotide. A further preferred embodiment of the method comprises the use of blocker oligonucleotides (the HeavyMethyl™ assay). The use of such blocker oligonucleotides has been described by Yu et al., BioTechniques 23:714-720, 1997. Blocking probe oligonucleotides are hybridized to the bisulfite treated nucleic acid concurrently with the PCR primers. PCR amplification of the nucleic acid is terminated at the 5′ position of the blocking probe, such that amplification of a nucleic acid is suppressed where the complementary sequence to the blocking probe is present. The probes may be designed to hybridize to the bisulfite treated nucleic acid in a methylation status specific manner. For example, for detection of methylated nucleic acids within a population of unmethylated nucleic acids, suppression of the amplification of nucleic acids which are unmethylated at the position in question would be carried out by the use of blocking probes comprising a ‘CpA’ or ‘TpA’ at the position in question, as opposed to a ‘CpG’ if the suppression of amplification of methylated nucleic acids is desired.

For PCR methods using blocker oligonucleotides, efficient disruption of polymerase-mediated amplification requires that blocker oligonucleotides not be elongated by the polymerase. Preferably, this is achieved through the use of blockers that are 3′-deoxyoligonucleotides, or oligonucleotides derivitized at the 3′ position with other than a “free” hydroxyl group. For example, 3′-O-acetyl oligonucleotides are representative of a preferred class of blocker molecule.

Additionally, polymerase-mediated decomposition of the blocker oligonucleotides should be precluded. Preferably, such preclusion comprises either use of a polymerase lacking 5′-3′ exonuclease activity, or use of modified blocker oligonucleotides having, for example, thioate bridges at the 5′-terminii thereof that render the blocker molecule nuclease-resistant. Particular applications may not require such 5′ modifications of the blocker. For example, if the blocker- and primer-binding sites overlap, thereby precluding binding of the primer (e.g., with excess blocker), degradation of the blocker oligonucleotide will be substantially precluded. This is because the polymerase will not extend the primer toward, and through (in the 5′-3′ direction) the blocker—a process that normally results in degradation of the hybridized blocker oligonucleotide.

A particularly preferred blocker/PCR embodiment, for purposes of the present invention and as implemented herein, comprises the use of peptide nucleic acid (PNA) oligomers as blocking oligonucleotides. Such PNA blocker oligomers are ideally suited, because they are neither decomposed nor extended by the polymerase.

Preferably, therefore, the base sequence of said blocking oligonucleotides comprise a sequence having a length of at least 9, 16, or 20 nucleotides which hybridizes to a treated nucleic acid sequence according to one of SEQ ID NOs. 2-5 preferably SEQ ID NOs. 7-10, and more preferably SEQ ID NO.s: 12-15 and sequences complementary thereto, wherein the base sequence of said oligonucleotides comprises at least one CpG, TpG or CpA dinucleotide. It is particularly preferred that the base sequence of said blocking oligonucleotides comprise a sequence having a length of at least 9, 16, or 20 nucleotides which hybridizes to a treated nucleic acid sequence according to one of SEQ ID NOs. 2-5 preferably SEQ ID NOs. 7-10, and more preferably SEQ ID NO.s: 12-15 and sequences complementary thereto, wherein the base sequence of said oligonucleotides comprises at least one TpG or CpA dinucleotide.

The fragments obtained by means of the amplification can carry a directly or indirectly detectable label. Preferred are labels in the form of fluorescence labels, radionuclides, or detachable molecule fragments having a typical mass which can be detected in a mass spectrometer. Where said labels are mass labels, it is preferred that the labelled amplificates have a single positive or negative net charge, allowing for better delectability in the mass spectrometer. The detection may be carried out and visualized by means of, e.g., matrix assisted laser desorption/ionization mass spectrometry (MALDI) or using electron spray mass spectrometry (ESI).

Matrix Assisted Laser Desorption/Ionization Mass Spectrometry (MALDI-TOF) is a very efficient development for the analysis of biomolecules (Karas & Hillenkamp, Anal Chem., 60:2299-301, 1988). An analyte is embedded in a light-absorbing matrix. The matrix is evaporated by a short laser pulse thus transporting the analyte molecule into the vapor phase in an unfragmented manner. The analyte is ionized by collisions with matrix molecules. An applied voltage accelerates the ions into a field-free flight tube. Due to their different masses, the ions are accelerated at different rates. Smaller ions reach the detector sooner than bigger ones. MALDI-TOF spectrometry is well suited to the analysis of peptides and proteins. The analysis of nucleic acids is somewhat more difficult (Gut & Beck, Current Innovations and Future Trends, 1:147-57, 1995). The sensitivity with respect to nucleic acid analysis is approximately 100-times less than for peptides, and decreases disproportionally with increasing fragment size. Moreover, for nucleic acids having a multiply negatively charged backbone, the ionization process via the matrix is considerably less efficient. In MALDI-TOF spectrometry, the selection of the matrix plays an eminently important role. For desorption of peptides, several very efficient matrixes have been found which produce a very fine crystallisation. There are now several responsive matrixes for DNA, however, the difference in sensitivity between peptides and nucleic acids has not been reduced. This difference in sensitivity can be reduced, however, by chemically modifying the DNA in such a manner that it becomes more similar to a peptide. For example, phosphorothioate nucleic acids, in which the usual phosphates of the backbone are substituted with thiophosphates, can be converted into a charge-neutral DNA using simple alkylation chemistry (Gut & Beck, Nucleic Acids Res. 23: 1367-73, 1995). The coupling of a charge tag to this modified DNA results in an increase in MALDI-TOF sensitivity to the same level as that found for peptides. A further advantage of charge tagging is the increased stability of the analysis against impurities, which makes the detection of unmodified substrates considerably more difficult.

In the fourth step of the method, the amplificates obtained during the third step of the method are analyzed in order to ascertain the methylation status of the CpG dinucleotides prior to the treatment.

In embodiments where the amplificates were obtained by means of MSP amplification, the presence, absence or class of an amplificate is in itself indicative of the methylation state of the CpG positions covered by the primer, according to the base sequences of said primer.

Amplificates obtained by means of both standard and methylation specific PCR may be further analyzed by means of based-based methods such as, but not limited to, array technology and probe based technologies as well as by means of techniques such as sequencing and template directed extension.

In one embodiment of the method, the amplificates synthesised in step three are subsequently hybridized to an array or a set of oligonucleotides and/or PNA probes. In this context, the hybridization takes place in the following manner: the set of probes used during the hybridization is preferably composed of at least 2 oligonucleotides or PNA-oligomers; in the process, the amplificates serve as probes which hybridize to oligonucleotides previously bonded to a solid phase; the non-hybridized fragments are subsequently removed; said oligonucleotides contain at least one base sequence having a length of at least 9, 16, or 20 nucleotides which is reverse complementary or identical to a segment of the base sequences specified in the present Sequence Listing; and the segment comprises at least one CpG, TpG or CpA dinucleotide. The hybridizing portion of the hybridizing nucleic acids is typically at least 9, 15, 20, 25, 30 or 35 nucleotides in length. However, longer molecules have inventive utility, and are thus within the scope of the present invention.

In a preferred embodiment, said dinucleotide is present in the central third of the oligomer. For example, wherein the oligomer comprises one CpG dinucleotide, said dinucleotide is preferably the fifth to ninth nucleotide from the 5′-end of a 13-mer. One oligonucleotide exists for the analysis of each CpG dinucleotide within a sequence SEQ ID NO.1 preferably SEQ ID NO. 6, and more preferably SEQ ID NO. 11 and the equivalent positions within SEQ ID NOs. 2-5, preferably SEQ ID NOs. 7-10, and more preferably SEQ ID NO.s: 12-15. Said oligonucleotides may also be present in the form of peptide nucleic acids. The non-hybridized amplificates are then removed. The hybridized amplificates are then detected. In this context, it is preferred that labels attached to the amplificates are identifiable at each position of the solid phase at which an oligonucleotide sequence is located.

In yet a further embodiment of the method, the genomic methylation status of the CpG positions may be ascertained by means of oligonucleotide probes (as detailed above) that are hybridized to the bisulfite treated DNA concurrently with the PCR amplification primers (wherein said primers may either be methylation specific or standard).

A particularly preferred embodiment of this method is the use of fluorescence-based Real Time Quantitative PCR (Heid et al., Genome Res. 6:986-994, 1996; also see U.S. Pat. No. 6,331,393) employing a dual-labelled fluorescent oligonucleotide probe (TaqMan™ PCR, using an ABI Prism 7700 Sequence Detection System, Perkin Elmer Applied Biosystems, Foster City, Calif.). The TaqMan™ PCR reaction employs the use of a non-extendible interrogating oligonucleotide, called a TaqMan™ probe, which, in preferred embodiments, is designed to hybridise to a CpG-rich sequence located between the forward and reverse amplification primers. The TaqMan™ probe further comprises a fluorescent “reporter moiety” and a “quencher moiety” covalently bound to linker moieties (e.g., phosphoramidites) attached to the nucleotides of the TaqMan™ oligonucleotide. For analysis of methylation within nucleic acids subsequent to bisulfite treatment, it is required that the probe be methylation specific, as described in U.S. Pat. No. 6,331,393, (hereby incorporated by reference in its entirety) also known as the MethyLight™ assay. Variations on the TaqMan™ detection methodology that are also suitable for use with the described invention include the use of dual-probe technology (LightCycler™) or fluorescent amplification primers (Sunrise™ technology). Both these techniques may be adapted in a manner suitable for use with bisulfite treated DNA, and moreover for methylation analysis within CpG dinucleotides.

In a further preferred embodiment of the method, the fourth step of the method comprises the use of template-directed oligonucleotide extension, such as MS-SNuPE as described by Gonzalgo & Jones, Nucleic Acids Res. 25:2529-2531, 1997.

In yet a further embodiment of the method, the fourth step of the method comprises sequencing and subsequent sequence analysis of the amplificate generated in the third step of the method (Sanger F., et al., Proc Natl Acad Sci USA 74:5463-5467, 1977).

Bisulfite modification of DNA is an art-recognized tool used to assess CpG methylation status. The most frequently used method for analyzing DNA for the presence of 5-methylcytosine is based upon the reaction of bisulfite with cytosine whereby, upon subsequent alkaline hydrolysis, cytosine is converted to uracil which corresponds to thymine in its base pairing behavior. Significantly, however, 5-methylcytosine remains unmodified under these conditions. Consequently, the original DNA is converted in such a manner that methylcytosine, which originally could not be distinguished from cytosine by its hybridization behavior, can now be detected as the only remaining cytosine using standard, art-recognized molecular biological techniques, for example, by amplification and hybridization, or by sequencing. All of these techniques are based on differential base pairing properties, which can now be fully exploited.

An overview of art-recognized methods for detecting 5-methylcytosine is provided by Rein, T., et al., Nucleic Acids Res., 26:2255, 1998.

The bisulfite technique, barring few exceptions (e.g., Zeschnigk M, et al., Eur J Hum Genet. 5:94-98, 1997), is currently only used in research. In general, short, specific fragments of a known gene are amplified subsequent to a bisulfite treatment, and either completely sequenced (Olek & Walter, Nat Genet. 1997 17:275-6, 1997), subjected to one or more primer extension reactions (Gonzalgo & Jones, Nucleic Acids Res., 25:2529-31, 1997; WO 95/00669; U.S. Pat. No. 6,251,594) to analyse individual cytosine positions, or treated by enzymatic digestion (Xiong & Laird, Nucleic Acids Res., 25:2532-4, 1997). Detection by hybridisation has also been described in the art (Olek et al., WO 99/28498). Additionally, use of the bisulfite technique for methylation detection with respect to individual genes has been described (Grigg & Clark, Bioessays, 16:431-6, 1994; Zeschnigk M, et al., Hum Mol Genet., 6:387-95, 1997; Feil R, et al., Nucleic Acids Res., 22:695-, 1994; Martin V, et al., Gene, 157:261-4, 1995; WO 9746705 and WO 9515373).

In further embodiment, the method of the invention is enabled by means of any analysis of the expression of an RNA transcribed therefrom or polypeptide or protein translated from said RNA, preferably by means of mRNA expression analysis or polypeptide expression analysis. However, in the most preferred embodiment of the invention predicting, prognosing, and/or monitoring therapeutic efficacy of cancer therapy on a subject having malignant tumor or cell proliferative disorder, is enabled by means of analysis of the methylation status of TFAP2E, and/or promoter or regulatory elements thereof.

Accordingly the present invention also provides predicting, prognostic and/or monitoring assays and methods, both quantitative and qualitative for detecting the expression of TFAP2E in a subject and predicting, prognosing, and/or monitoring therapeutic efficacy of cancer therapy on a subject having malignant tumor or cell proliferative disorder in said subject.

Aberrant expression of mRNA transcribed from TFAP2E is associated with the progression of having malignant tumor or cell proliferative disorder in a subject.

To detect the presence of mRNA encoding a gene or genomic sequence, a sample is obtained from the subject. The sample may be any suitable sample comprising cellular matter of the tumor. Suitable sample types include metastatic cells, cell lines, histological slides, biopsies, paraffin-embedded tissue, frozen tissue, formalin fixed tissue, body fluids, stool, colonic effluent , urine, blood plasma, blood serum, whole blood, isolated blood cells, cells isolated from the blood and combinations thereof. and all possible combinations thereof.

The sample may be treated to extract the RNA contained therein. The resulting nucleic acid from the sample is then analysed. Many techniques are known in the state of the art for determining absolute and relative levels of gene expression, commonly used techniques suitable for use in the present invention include in situ hybridisation (e.g. FISH), Northern analysis, RNase protection assays (RPA), microarrays and PCR-based techniques, such as quantitative PCR and differential display PCR or any other nucleic acid detection method.

Particularly preferred is the use of the reverse transcription/polymerisation chain reaction technique (RT-PCR). The method of RT-PCR is well known in the art (for example, see Watson and Fleming, supra).

The RT-PCR method can be performed as follows. Total cellular RNA is isolated by, for example, the standard guanidium isothiocyanate method and the total RNA is reverse transcribed. The reverse transcription method involves synthesis of DNA on a template of RNA using a reverse transcriptase enzyme and a 3′ end oligonucleotide dT primer and/or random hexamer primers. The cDNA thus produced is then amplified by means of PCR. (Belyaysky et al, Nucl Acid Res 17:2919-2932, 1989; Krug and Berger, Methods in Enzymology, Academic Press, N.Y., Vol. 152, pp. 316-325, 1987 which are incorporated by reference). Further preferred is the “Real-time” variant of RT-PCR, wherein the PCR product is detected by means of hybridisation probes (e.g. TaqMan, LightCycler, Molecular Beacons & Scorpion) or SYBR green. The detected signal from the probes or SYBR green is then quantitated either by reference to a standard curve or by comparing the Ct values to that of a calibration standard. Analysis of housekeeping genes is often used to normalize the results.

In Northern blot analysis total or poly(A)+mRNA is run on a denaturing agarose gel and detected by hybridisation to a labelled probe in the dried gel itself or on a membrane. The resulting signal is proportional to the amount of target RNA in the RNA population.

Comparing the signals from two or more cell populations or tissues reveals relative differences in gene expression levels. Absolute quantitation can be performed by comparing the signal to a standard curve generated using known amounts of an in vitro transcript corresponding to the target RNA. Analysis of housekeeping genes, genes whose expression levels are expected to remain relatively constant regardless of conditions, is often used to normalize the results, eliminating any apparent differences caused by unequal transfer of RNA to the membrane or unequal loading of RNA on the gel.

The first step in Northern analysis is isolating pure, intact RNA from the cells or tissue of interest. Because Northern blots distinguish RNAs by size, sample integrity influences the degree to which a signal is localized in a single band. Partially degraded RNA samples will result in the signal being smeared or distributed over several bands with an overall loss in sensitivity and possibly an erroneous interpretation of the data. In Northern blot analysis, DNA, RNA and oligonucleotide probes can be used and these probes are preferably labelled (e.g. radioactive labels, mass labels or fluorescent labels). The size of the target RNA, not the probe, will determine the size of the detected band, so methods such as random-primed labelling, which generates probes of variable lengths, are suitable for probe synthesis. The specific activity of the probe will determine the level of sensitivity, so it is preferred that probes with high specific activities, are used.

In an RNase protection assay, the RNA target and an RNA probe of a defined length are hybridised in solution. Following hybridisation, the RNA is digested with RNases specific for single-stranded nucleic acids to remove any unhybridized, single-stranded target RNA and probe. The RNases are inactivated, and the RNA is separated e.g. by denaturing polyacrylamide gel electrophoresis. The amount of intact RNA probe is proportional to the amount of target RNA in the RNA population. RPA can be used for relative and absolute quantitation of gene expression and also for mapping RNA structure, such as intron/exon boundaries and transcription start sites. The RNase protection assay is preferable to Northern blot analysis as it generally has a lower limit of detection.

The antisense RNA probes used in RPA are generated by in vitro transcription of a DNA template with a defined endpoint and are typically in the range of 50-600 nucleotides. The use of RNA probes that include additional sequences not homologous to the target RNA allows the protected fragment to be distinguished from the full-length probe. RNA probes are typically used instead of DNA probes due to the ease of generating single-stranded RNA probes and the reproducibility and reliability of RNA:RNA duplex digestion with RNases (Ausubel et al. 2003), particularly preferred are probes with high specific activities.

Particularly preferred is the use of microarrays. The microarray analysis process can be divided into two main parts. First is the immobilization of known gene sequences onto glass slides or other solid support followed by hybridisation of the fluorescently labelled cDNA (comprising the sequences to be interrogated) to the known genes immobilized on the glass slide (or other solid phase). After hybridisation, arrays are scanned using a fluorescent microarray scanner. Analysing the relative fluorescent intensity of different genes provides a measure of the differences in gene expression.

DNA arrays can be generated by immobilizing presynthesized oligonucleotides onto prepared glass slides or other solid surfaces. In this case, representative gene sequences are manufactured and prepared using standard oligonucleotide synthesis and purification methods. These synthesized gene sequences are complementary to the RNA transcript(s) of TFAP2E and tend to be shorter sequences in the range of 25-70 nucleotides. Alternatively, immobilized oligos can be chemically synthesized in situ on the surface of the slide. In situ oligonucleotide synthesis involves the consecutive addition of the appropriate nucleotides to the spots on the microarray; spots not receiving a nucleotide are protected during each stage of the process using physical or virtual masks. Preferably said synthesized nucleic acids are locked nucleic acids.

In expression profiling microarray experiments, the RNA templates used are representative of the transcription profile of the cells or tissues under study. RNA is first isolated from the cell populations or tissues to be compared. Each RNA sample is then used as a template to generate fluorescently labelled cDNA via a reverse transcription reaction. Fluorescent labelling of the cDNA can be accomplished by either direct labelling or indirect labelling methods. During direct labelling, fluorescently modified nucleotides (e.g., Cy®3- or Cy®5-dCTP) are incorporated directly into the cDNA during the reverse transcription. Alternatively, indirect labelling can be achieved by incorporating aminoallyl-modified nucleotides during cDNA synthesis and then conjugating an N-hydroxysuccinimide (NHS)-ester dye to the aminoallyl-modified cDNA after the reverse transcription reaction is complete. Alternatively, the probe may be unlabelled, but may be detectable by specific binding with a ligand which is labelled, either directly or indirectly. Suitable labels and methods for labelling ligands (and probes) are known in the art, and include, for example, radioactive labels which may be incorporated by known methods (e.g., nick translation or kinasing). Other suitable labels include but are not limited to biotin, fluorescent groups, chemiluminescent groups (e.g., dioxetanes, particularly triggered dioxetanes), enzymes, antibodies, and the like.

To perform differential gene expression analysis, cDNA generated from different RNA samples are labelled with Cy®3. The resulting labelled cDNA is purified to remove unincorporated nucleotides, free dye and residual RNA. Following purification, the labelled cDNA samples are hybridised to the microarray. The stringency of hybridisation is determined by a number of factors during hybridisation and during the washing procedure, including temperature, ionic strength, length of time and concentration of formamide. These factors are outlined in, for example, Sambrook et al. (Molecular Cloning: A Laboratory Manual, 2nd ed., 1989). The microarray is scanned post-hybridisation using a fluorescent microarray scanner. The fluorescent intensity of each spot indicates the level of expression of the analysed gene; bright spots correspond to strongly expressed genes, while dim spots indicate weak expression.

Once the images are obtained, the raw data must be analysed. First, the background fluorescence must be subtracted from the fluorescence of each spot. The data is then normalized to a control sequence, such as exogenously added nucleic acids (preferably RNA or DNA), or a housekeeping gene panel to account for any non-specific hybridisation, array imperfections or variability in the array set-up, cDNA labelling, hybridisation or washing. Data normalization allows the results of multiple arrays to be compared.

### Methylation Sensitive Restriction Enzyme Analysis

In an alternative embodiment of the invention the above described second step may be carried out by means of methylation sensitive or methylation specific restriction enzyme analysis. Methods are known in the art wherein a methylation sensitive restriction enzyme reagent, or a series of restriction enzyme reagents comprising methylation sensitive restriction enzyme reagents that distinguishes between methylated and non-methylated CpG dinucleotides within a target region are utilized in determining methylation, for example but not limited to DMH.

In a preferred embodiment, the DNA may be cleaved prior to treatment with methylation sensitive restriction enzymes. Such methods are known in the art and may include both physical and enzymatic means. Particularly preferred is the use of one or a plurality of restriction enzymes which are not methylation sensitive, and whose recognition sites are AT rich and do not comprise CG dinucleotides. The use of such enzymes enables the conservation of CpG islands and CpG rich regions in the fragmented DNA. The non-methylation-specific restriction enzymes are preferably selected from the group consisting of MseI, BfaI, Csp6I, Tru1I, Tvu1I, Tru9I, Tvu9I, MaeI and XspI. Particularly preferred is the use of two or three such enzymes. Particularly preferred is the use of a combination of MseI, BfaI and Csp6I.

The fragmented DNA may then be ligated to adaptor oligonucleotides in order to facilitate subsequent enzymatic amplification. The ligation of oligonucleotides to blunt and sticky ended DNA fragments is known in the art, and is carried out by means of dephosphorylation of the ends (e.g. using calf or shrimp alkaline phosphatase) and subsequent ligation using ligase enzymes (e.g. T4 DNA ligase) in the presence of dATPs. The adaptor oligonucleotides are typically at least 18 base pairs in length.

In the third step, the DNA (or fragments thereof) is then digested with one or more methylation sensitive restriction enzymes. The digestion is carried out such that hydrolysis of the DNA at the restriction site is informative of the methylation status of a specific CpG dinucleotide of TFAP2E.

Preferably, the methylation-specific restriction enzyme is selected from the group consisting of Bsi E1, Hga I HinP1, Hpy99I, Ava I, Bce AI, Bsa HI, BisI, BstUI, BshI236I, AccII, BstFNI, McrBC, GlaI, MvnI, HpaII (HapII), HhaI, Acil, SmaI, HinP1I, HpyCH4IV, EagI and mixtures of two or more of the above enzymes. Preferred is a mixture containing the restriction enzymes BstUI, HpaII, HpyCH4IV and HinP1I.

In the fourth step, which is optional but a preferred embodiment, the restriction fragments are amplified. This is preferably carried out using a polymerase chain reaction, and said amplificates may carry suitable detectable labels as discussed above, namely fluorophore labels, radionuclides and mass labels. Particularly preferred is amplification by means of an amplification enzyme and at least two primers comprising, in each case a contiguous sequence of nucleotides that is complementary to, or hybridizes under moderately stringent or stringent conditions to SEQ ID NO. 1 preferably SEQ ID NO. 6, and more preferably SEQ ID NO.11, and complements thereof. Preferably said contiguous sequence is at least 16, 20 or 25 nucleotides in length. In an alternative embodiment said primers may be complementary to any adaptors linked to the fragments.

In the fifth step the amplificates are detected. The detection may be by any means standard in the art, for example, but not limited to, gel electrophoresis analysis, hybridisation analysis, incorporation of detectable tags within the PCR products, DNA array analysis, MALDI or ESI analysis. Preferably said detection is carried out by hybridisation to at least one nucleic acid or peptide nucleic acid comprising in each case a contiguous sequence at least 16 nucleotides in length that is complementary to, or hybridizes under moderately stringent or stringent conditions to a sequence selected from the group consisting SEQ ID NO. 1 preferably SEQ ID NO. 6, and more preferably SEQ ID NO.11, and complements thereof. Preferably said contiguous sequence is at least 16, 20 or 25 nucleotides in length.

Subsequent to the determination of the methylation state or level of the genomic nucleic acids the predicting, prognosing, and/or monitoring therapeutic efficacy of cancer therapy on a subject having malignant tumor or cell proliferative disorder, is deduced based upon the methylation state or level of at least one CpG dinucleotide sequence of TFAP2E, or an average, or a value reflecting an average methylation state of a plurality of CpG dinucleotide sequences of SEQ ID NO. 1 wherein methylation is associated with the predicting, prognosing, and/or monitoring therapeutic efficacy of cancer therapy on a subject having malignant tumor or cell proliferative disorder. Said methylation is in particular associated with the subject's predicting, prognosing, and/or monitoring subsequent to chemotherapy.

### Cut Off Value

Wherein said methylation is determined by quantitative means the cut-off value for determining said presence of methylation is preferably zero (i.e. wherein a sample displays any degree of methylation it is determined as having a methylated status at the analyzed CpG position). Nonetheless, it is foreseen that the person skilled in the art may wish to adjust said cut-off value in order to provide an assay of a particularly preferred sensitivity or specificity. Accordingly said cut-off value may be increased (thus increasing the specificity), said cut off value may be within a range selected form the group consisting of 0%-5%, 5%-10%, 10%-15%, 15%-20%, 20%-30% and 30%-50%. Particularly preferred are cut-off values that are at least 0.1%, 1%, 10%, 15%, 25%, and 30%.

Upon determination of the methylation and/or expression of the TFAP2E the predicting, prognosing, and/or monitoring of the subject is determined. Hypermethylation and/or decreased expression of TFAP2E is indicative of resistance to cancer therapy while hypomethylation and/or increased expression of TFAP2E is indicative of sensitivity to said cancer therapy.

### FURTHER EMBODIMENTS

The disclosed invention provides treated nucleic acids, derived from genomic SEQ ID NO. 1, preferably SEQ ID NO. 6, and more preferably SEQ ID NO.: 11, wherein the treatment is suitable to convert at least one unmethylated cytosine base of the genomic DNA sequence to uracil or another base that is detectably dissimilar to cytosine in terms of hybridization. The genomic sequences in question may comprise one, or more consecutive methylated CpG positions. Said treatment preferably comprises use of a reagent selected from the group consisting of bisulfite, hydrogen sulfite, disulfite, and combinations thereof. In a preferred embodiment of the invention, the invention provides a non-naturally occurring modified nucleic acid comprising a sequence of contiguous nucleotide bases of a sequence selected from the group consisting of SEQ ID NOS. 2-5, preferably SEQ ID NO. 7-10, and more preferably SEQ ID NO.: 12-15. In further preferred embodiments of the invention said nucleic acid is at least 50, 100, 150, 200, 250 or 500 base pairs in length of a segment of the nucleic acid sequence disclosed in SEQ ID NOs. 2-5 preferably SEQ ID NO. 7-10, and more preferably SEQ ID NO.: 12-15. Particularly preferred is a nucleic acid molecule that is identical or complementary to all or a portion of the sequences SEQ ID NOs. 2-5, preferably SEQ ID NO. 7-10, and more preferably SEQ ID NO.: 12-15 but not SEQ ID NOs. 1, 6, or 11 or other naturally occurring DNA.

It is preferred that said sequence comprises at least one CpG, TpA or CpA dinucleotide and sequences complementary thereto. The sequences of SEQ ID NOS. 2-5, 7-10, 12-15 provide non-naturally occurring modified versions of the nucleic acid according to SEQ ID NO. 1, 6, 11, respectively, wherein the modification of each genomic sequence results in the synthesis of a nucleic acid having a sequence that is unique and distinct from said genomic sequence as follows. For each sense strand genomic DNA, e.g., SEQ ID NO. 1, four converted versions are disclosed. A first version wherein “C” is converted to “T,” but “CpG” remains “CpG” (i.e., corresponds to case where, for the genomic sequence, all “C” residues of CpG dinucleotide sequences are methylated and are thus not converted); a second version discloses the complement of the disclosed genomic DNA sequence (i.e. antisense strand), wherein “C” is converted to “T,” but “CpG” remains “CpG” (i.e., corresponds to case where, for all “C” residues of CpG dinucleotide sequences are methylated and are thus not converted). The ‘upmethylated’ converted sequences of SEQ ID NO. 1 correspond to SEQ ID NOs. 2 and 4. A third chemically converted version of each genomic sequences is provided, wherein “C” is converted to “T” for all “C” residues, including those of “CpG” dinucleotide sequences (i.e., corresponds to case where, for the genomic sequences, all “C” residues of CpG dinucleotide sequences are unmethylated); a final chemically converted version of each sequence, discloses the complement of the disclosed genomic DNA sequence (i.e. antisense strand), wherein “C” is converted to “T” for all “C” residues, including those of “CpG” dinucleotide sequences (i.e., corresponds to case where, for the complement (antisense strand) of each genomic sequence, all “C” residues of CpG dinucleotide sequences are unmethylated). For example, the ‘downmethylated’ converted sequences of SEQ ID NO. 1 corresponds to SEQ ID NOs. 3 and 5.

In an alternative preferred embodiment, the invention further provides oligonucleotides or oligomers suitable for use in the methods of the invention for detecting the cytosine methylation state within genomic or treated (chemically modified) DNA, according to SEQ ID NOs. 1-16. Said oligonucleotide or oligomer nucleic acids provide novel prognostic means. Said oligonucleotide or oligomer comprising a nucleic acid sequence having a length of preferably nine (9), sixteen (16), or twenty (20) nucleotides which is identical to, hybridizes, under moderately stringent or stringent conditions (as defined herein above), to a treated nucleic acid sequence according to SEQ ID NOs. 2-5 preferably SEQ ID NO. 7-10, and more Preferably SEQ ID NO.: 12-15 and/or sequences complementary thereto, or to a genomic sequence according to SEQ ID NO. 1, 6, and 11 respectively and/or sequences complementary thereto. Thus, the present invention includes nucleic acid molecules (e.g., oligonucleotides and peptide nucleic acid (PNA) molecules (PNA-oligomers)) that hybridize under moderately stringent and/or stringent hybridization conditions to all or a portion of the sequences SEQ ID No. 2-5 preferably SEQ ID NO. 7-10, and more Preferably SEQ ID NO.: 12-15 or to the complements thereof. The identical or hybridizing portion of the hybridizing nucleic acids is typically at least 9, 16, 20, 25, 30 or 35 nucleotides in length. However, longer molecules have inventive utility, and are thus within the scope of the present invention.

Preferably, the hybridizing portion of the inventive hybridizing nucleic acids is at least 95%, or at least 98%, or 100% identical to the sequence, or to a portion thereof of SEQ ID NO. 2-5 preferably SEQ ID NO. 7-10, and more Preferably SEQ ID NO.: 12-15, or to the complements thereof.

Hybridizing nucleic acids of the type described herein can be used, for example, as a primer (e.g., a PCR primer), or a prognostic probe or primer. Preferably, hybridization of the oligonucleotide probe to a nucleic acid sample is performed under stringent conditions and the probe is 100% identical to the target sequence. Nucleic acid duplex or hybrid stability is expressed as the melting temperature or Tm, which is the temperature at which a probe dissociates from a target DNA. This melting temperature is used to define the required stringency conditions.

For target sequences that are related and substantially identical to the corresponding sequence of SEQ ID NO. 1, preferably SEQ ID NO. 6, and more Preferably SEQ ID NO.: 11 (such as allelic variants and SNPs), rather than identical, it is useful to first establish the lowest temperature at which only homologous hybridization occurs with a particular concentration of salt (e.g., SSC or SSPE). Then, assuming that 1% mismatching results in a 1° C. decrease in the Tm, the temperature of the final wash in the hybridization reaction is reduced accordingly (for example, if sequences having >95% identity with the probe are sought, the final wash temperature is decreased by 5° C.). In practice, the change in Tm can be between 0.5° C. and 1.5° C. per 1% mismatch.

Examples of inventive oligonucleotides of length X (in nucleotides), as indicated by polynucleotide positions with reference to, e.g., SEQ ID NO: 1, include those corresponding to sets (sense and antisense sets) of consecutively overlapping oligonucleotides of length X, where the oligonucleotides within each consecutively overlapping set (corresponding to a given X value) are defined as the finite set of Z oligonucleotides from nucleotide positions:

n to (n+(X−1));
where n=1, 2, 3, . . . (Y−(X−1));
where Y equals the length (nucleotides or base pairs) of SEQ ID NO: 1 (6354);
where X equals the common length (in nucleotides) of each oligonucleotide in the set (e.g., X=20 for a set of consecutively overlapping 20-mers); and
where the number (Z) of consecutively overlapping oligomers of length X for a given SEQ ID NO 1 of length Y is equal to Y−(X−1). For example Z=6354−19=6335 for either sense or antisense sets of SEQ ID NO: 1, where X=20.

Preferably, the set is limited to those oligomers that comprise at least one CpG, TpG or CpA dinucleotide.

Examples of inventive 20-mer oligonucleotides include the following set of 6335 oligomers (and the antisense set complementary thereto), indicated by polynucleotide positions with reference to SEQ ID NO: 1:

1-20, 2-21, 3-22, 4-23, 5-24, . . . and 6335-6354.

Preferably, the set is limited to those oligomers that comprise at least one CpG, TpG or CpA dinucleotide.

Likewise, examples of inventive 25-mer oligonucleotides include the following set of 6330 oligomers (and the antisense set complementary thereto), indicated by polynucleotide positions with reference to SEQ ID NO: 1:

1-25, 2-26, 3-27, 4-28, 5-29, . . . and 6330-6354.

Preferably, the set is limited to those oligomers that comprise at least one CpG, TpG or CpA dinucleotide.

The present invention encompasses, for each of SEQ ID Nos. 1-6 (sense and antisense), multiple consecutively overlapping sets of oligonucleotides or modified oligonucleotides of length X, where, e.g., X=9, 10, 17, 20, 22, 23, 25, 27, 30 or 35 nucleotides.

The oligonucleotides or oligomers according to the present invention constitute effective tools useful to ascertain genetic and epigenetic parameters of the genomic sequence corresponding to SEQ ID NO. 1 preferably SEQ ID NO. 6, and more Preferably SEQ ID NO.: 11. Preferred sets of such oligonucleotides or modified oligonucleotides of length X are those consecutively overlapping sets of oligomers corresponding to SEQ ID NO. 2-5 preferably SEQ ID NO. 7-10, and more Preferably SEQ ID NO.: 12-15 (and to the complements thereof). Preferably, said oligomers comprise at least one CpG, TpG or CpA dinucleotide.

Particularly preferred oligonucleotides or oligomers according to the present invention are those in which the cytosine of the CpG dinucleotide (or of the corresponding converted TpG or CpA dinculeotide) sequences is within the middle third of the oligonucleotide; that is, where the oligonucleotide is, for example, 13 bases in length, the CpG, TpG or CpA dinucleotide is positioned within the fifth to ninth nucleotide from the 5′-end.

The oligonucleotides of the invention can also be modified by chemically linking the oligonucleotide to one or more moieties or conjugates to enhance the activity, stability or detection of the oligonucleotide. Such moieties or conjugates include chromophores, fluorophors, lipids such as cholesterol, cholic acid, thioether, aliphatic chains, phospholipids, polyamines, polyethylene glycol (PEG), palmityl moieties, and others as disclosed in, for example, U.S. Pat. Nos. 5,514,758, 5,565,552, 5,567,810, 5,574,142, 5,585,481, 5,587,371, 5,597,696 and 5,958,773. The probes may also exist in the form of a PNA (peptide nucleic acid) which has particularly preferred pairing properties. Thus, the oligonucleotide may include other appended groups such as peptides, and may include hybridization-triggered cleavage agents (Krol et al., BioTechniques 6:958-976, 1988) or intercalating agents (Zon, Pharm. Res. 5:539-549, 1988). To this end, the oligonucleotide may be conjugated to another molecule, e.g., a chromophore, fluorophor, peptide, hybridization-triggered cross-linking agent, transport agent, hybridization-triggered cleavage agent, etc.

The oligonucleotide may also comprise at least one art-recognized modified sugar and/or base moiety, or may comprise a modified backbone or non-natural internucleoside linkage.

The oligonucleotides or oligomers according to particular embodiments of the present invention are typically used in ‘sets,’ which contain at least one oligomer for analysis of each of the CpG dinucleotides of a genomic sequence selected from the group consisting SEQ ID NO. 1 preferably SEQ ID NO. 6, and more Preferably SEQ ID NO.: 11 and sequences complementary thereto, or to the corresponding CpG, TpG or CpA dinucleotide within a sequence of the treated nucleic acids according to SEQ ID NOs. 2-5, preferably SEQ ID NO. 7-10, and, more Preferably SEQ ID NO.: 12-15 and sequences complementary thereto. However, it is anticipated that for economic or other factors it may be preferable to analyse a limited selection of the CpG dinucleotides within said sequences, and the content of the set of oligonucleotides is altered accordingly.

Therefore, in particular embodiments, the present invention provides preferably a set of at least two (2) (oligonucleotides and/or PNA-oligomers) useful for detecting the cytosine methylation state in treated genomic DNA (SEQ ID NOs. 2-5 preferably SEQ ID NO. 7-10, and more Preferably SEQ ID NO.: 12-15), or in genomic DNA (SEQ ID NO. 1, 6, and 11 respectively) and sequences complementary thereto). These probes enable determination of the predicting, prognosing, and/or monitoring therapeutic efficacy of cancer therapy on a subject having having malignant tumor or cell proliferative disorder. The set of oligomers may also be used for detecting single nucleotide polymorphisms (SNPs) in treated genomic DNA (SEQ ID NOs. 2-5 preferably SEQ ID NO. 7-10, and more Preferably SEQ ID NO.: 12-15), or in genomic DNA (SEQ ID NO. 1, 6, 11 respectively) and sequences complementary thereto).

In preferred embodiments, at least one, and more preferably all members of a set of oligonucleotides is bound to a solid phase.

In further embodiments, the present invention provides preferably a set of at least two (2) oligonucleotides that are used as ‘primer’ oligonucleotides for amplifying DNA sequences of one of SEQ ID NO. 1-16 and sequences complementary thereto, or segments thereof.

It is anticipated that the oligonucleotides may constitute all or part of an “array” or “DNA chip” (i.e., an arrangement of different oligonucleotides and/or PNA-oligomers bound to a solid phase). Such an array of different oligonucleotide- and/or PNA-oligomer sequences can be characterized, for example, in that it is arranged on the solid phase in the form of a rectangular or hexagonal lattice. The solid-phase surface may be composed of silicon, glass, polystyrene, aluminium, steel, iron, copper, nickel, silver, or gold. Nitrocellulose as well as plastics such as nylon, which can exist in the form of pellets or also as resin matrices, may also be used. An overview of the Prior Art in oligomer array manufacturing can be gathered from a special edition of Nature Genetics (Nature Genetics Supplement, Volume 21, January 1999, and from the literature cited therein). Fluorescently labelled probes are often used for the scanning of immobilized DNA arrays. The simple attachment of Cy3 and Cy5 dyes to the 5′-OH of the specific probe are particularly suitable for fluorescence labels. The detection of the fluorescence of the hybridised probes may be carried out, for example, via a confocal microscope. Cy3 and Cy5 dyes, besides many others, are commercially available.

It is also anticipated that the oligonucleotides, or particular sequences thereof, may constitute all or part of an “virtual array” wherein the oligonucleotides, or particular sequences thereof, are used, for example, as ‘specifiers’ as part of, or in combination with a diverse population of unique labeled probes to analyze a complex mixture of analytes. Such a method, for example is described in US 2003/0013091 (U.S. Ser. No. 09/898,743, published 16 Jan. 2003). In such methods, enough labels are generated so that each nucleic acid in the complex mixture (i.e., each analyte) can be uniquely bound by a unique label and thus detected (each label is directly counted, resulting in a digital read-out of each molecular species in the mixture).

It is particularly preferred that the oligomers according to the invention are utilized for predicting, prognosing, and/or monitoring therapeutic efficacy of cancer therapy on a subject having malignant tumor or cell proliferative disorder. It is further particularly preferred that the oligomers according to the invention are utilized for determining the predicting, prognosing, and/or monitoring of said subject subsequent to chemotherapy.

Preferred is a method of predicting, prognosing, and/or monitoring therapeutic efficacy of cancer therapy on a subject having malignant tumor and/or cell proliferative disorder comprising:

a) determining the methylation and/or expression levels of TFAP2E in a biological sample isolated from said subject; and

b) predicting, prognosing, and/or monitoring therapeutic efficacy of cancer therapy on said subject based on step a),

wherein hypermethylation and/or decreased expression of TFAP2E is indicative of resistance to cancer therapy, while hypomethylation and/or increased expression of TFAP2E is indicative of sensitivity to cancer therapy.

Preferred is a method, wherein said predicting, prognosing, and/or monitoring is determined by detecting the presence, absence or amount of CpG methylation within TFAP2E or sequence thereof.

Preferred is a method, wherein said expression level is determined by detecting the presence, absence or level of mRNA transcribed from TFAP2E.

Preferred is a method, wherein said expression level is determined by detecting the presence, absence or level of a polypeptide encoded by TFAP2E or sequence thereof.

Preferred is a method, wherein said malignant tumor and/or cell proliferative disorder is colorectal, pancreatic, gastric, head and neck, breast, non-small-cell lung, leukaemia, and/or gynaecologic cancer.

Preferred is a method, wherein said cancer therapy comprises chemotherapy, polychemotherapy, chemoradiotherapy, treatments with chemotherapeutic agents, treatments with antimetabolites, pyrimidine antagonists, cytarabine, decitabine, floxuridine, capecitabine, or Gemcitabine and/or combination thereof.

Preferred is a method, wherein said cancer therapy is 5-Flurouracil based chemotherapy or polychemothrapies.

Preferred is a method, comprising contacting genomic DNA isolated from a biological sample obtained from said subject with at least one reagent, or series of reagents that distinguishes between methylated and non-methylated CpG dinucleotides within at least one target region of the genomic DNA, wherein the target region comprises, or hybridizes under stringent conditions to a sequence of contiguous nucleotides of SEQ ID NO: 1, wherein said contiguous nucleotides comprise at least one CpG dinucleotide sequence.

More preferred is a method which further comprises:
a. contacting the treated genomic DNA, or the treated fragment thereof, with an amplification enzyme and at least one oligonucleotide comprising a contiguous sequence of nucleotides that is complementary to, or hybridizes under moderately stringent or stringent conditions to a sequence selected from the group consisting of SEQ ID NOs: 2-5, and complements thereof, wherein the treated genomic DNA or the fragment thereof is either amplified to produce at least one amplificate, or is not amplified; and
b. determining, based on a presence, absence or amount of, or on a property of said amplificate, the methylation state or level of at least one CpG dinucleotide of SEQ ID NO: 1, or an average, or a value reflecting an average methylation state or level of a plurality of CpG dinucleotides of SEQ ID NO: 1, whereby predicting, prognosing, and/or monitoring of said subject is afforded.

More preferred is a method, wherein treating the genomic DNA, or the fragment thereof, comprises use of a reagent selected from the group comprising of bisulfite, hydrogen sulfite, disulfite, and combinations thereof.

Preferred is also a method, wherein the biological sample obtained from the subject is selected from the group comprising metastatic cells, cell lines, histological slides, biopsies, paraffin-embedded tissue, frozen tissue, formalin fixed tissue, body fluids, stool, colonic effluent , urine, blood plasma, blood serum, whole blood, isolated blood cells, cells isolated from the blood and combinations thereof

Preferred is also a method which further comprises:
c. digesting the genomic DNA, or a fragment thereof, isolated from a biological sample obtained from said subject with one or more methylation sensitive restriction enzymes;
d. contacting the DNA restriction enzyme digest of a), with an amplification enzyme and at least one oligonucleotide suitable for the amplification of a sequence comprising at least one CpG dinucleotide of SEQ ID NO: 1;
e. determining, based on a presence, absence or class of an amplificate the methylation state or level of at least one CpG dinucleotide of at least one sequence selected from the group consisting of SEQ ID NO: 1, and therefrom predicting, prognosing, and/or monitoring of said subject is afforded.

Another preferred aspect of the invention is a kit for predicting, prognosing, and/or monitoring therapeutic efficacy of cancer therapy in a subject having malignant tumor or cell proliferative disorder who has undergone or is undergoing chemotherapy and/or chemoradiotherapy, characterized in that said kit comprises (a) one or more reagents to convert unmethylated cytosine bases to uracil or to anther base that is detectably dissimilar to cytosine in terms of hybridization properties (b) at least one oligonucleotide or polynucleotide able to hybridize under stringent or moderately stringent conditions to at least one sequence selected from the group consisting of SEQ ID NOs.: 2-5 in order to detect the presence, absence or amount of CpG methylation within TFAP2E, c) containers suitable for containing the said one or more reagents and the biological sample of the patient; and optionally (d) instructions for use.

Another preferred aspect of the invention is a kit for predicting, prognosing, and/or monitoring therapeutic efficacy of cancer therapy in a subject having malignant tumor or cell proliferative disorder who has undergone or is undergoing chemotherapy and/or chemoradiotherapy, characterized in that said kit comprises (a) a methylation sensitive restriction enzyme reagent; (b) a container suitable for containing the said reagent and the biological sample of the patient; (c) at least one set of oligonucleotides one or a plurality of nucleic acids or peptide nucleic acids which are identical, are complementary, or hybridize under stringent or highly stringent conditions to a sequence of SEQ ID NO: 1 in order to detect the presence, absence or amount of CpG methylation within TFAP2E; and optionally (d) instructions for use.

Moreover, another preferred aspect of the invention is the use of a method according to claims 1 to 12, and/or a kit according to claim 13 or 14 in predicting, prognosing, and/or monitoring therapeutic efficacy of cancer therapy in a subject having malignant tumor or cell proliferative disorder.

TABLE 1
Genomic Sequences and Treated Variants Thereof According to the Invention

| | | Pretreated | Pretreated | Pretreated | Pretreated |
|---|---|---|---|---|---|
| | | methylated | unmethylated | methylated | unmethylated |
| | Genomic | sequence | sequence | strand | sequence |
| | SEQ ID | (sense) SEQ | (sense) SEQ ID | (antisense) SEQ | (antisense) SEQ |
| Gene | NO: | ID NO: | NO: | ID NO: | ID NO: |
| TFAP2E | 1 | 2 | 3 | 4 | 5 |
| TFAP2E | 6 | 7 | 8 | 9 | 10 |
| (793 nt- | | | | | |
| 2728 nt) | | | | | |
| TFAP2E | 11 | 12 | 13 | 14 | 15 |
| (1577 nt- | | | | | |
| 1717 nt) | | | | | |
| DKK4 | 16 | — | — | — | — |

TABLE 2
Primer and Probe sequences.

| | | | | | |
|---|---|---|---|---|---|
| | | | | Accession/ | |
| | | | Product | Location | |
| SEQ ID | | | length (when | (when | |
| NO. | GENE | Primer sequence | applicable) | applicable) | Usage |

TFAP2E

| | | | | | |
|---|---|---|---|---|---|
| 17 | Forward | CCGGACGCTTGCCTGCAGTC | | | MSP |
| | | | | | (Genomic) |
| 18 | Reverse | CCCAGAAGCGGCCTTCGCATC | | | MSP |
| | | | | | (Genomic) |
| 19 | Probe | AGCCCGAGAACGCCCACCGCAG | | | MSP |
| | | | | | (Genomic) |
| 20 | Forward | TCGGACGTTTGTTTGTAGTT | | | MSP |
| | | | | | (Treated) |
| 21 | Reverse | CCCAAAAACGACCTTCGCATC | | | MSP |
| | | | | | (Treated) |
| 22 | Probe | AGTTCGAGAACGTTTATCGTAG | | | MSP |
| | | | | | (Treated) |
| 23 | Reverse | CCGAACGCTTACCTACAATC | CpG Island | Intron3 | Methylight/ |
| | | | | | MS-HRM |
| 24 | Forward | TTTAGAAGCGGTTTTCGTATC | 139 bp | NC_000001.10 | Methylight/ |
| | | | | | MS-HRM |
| 25 | Probe | TTGCGGTGGGCGTTTTCGGGTT | 3′BHQ1- | | Methylight/ |
| | | | 5′FAM | | MS-HRM |
| 26 | Forward | TAGTTTGAAAGGATTTTTTTTAGT | | | BSP |
| | | | | | (Treated) |
| 27 | Reverse | ACTCTAATCAATATAAACTAAATTCA | | | BSP |
| | | | | | (Treated) |
| 28 | Reverse | CAACCTAAAAAAATCCTCCTCAAC | CpG Island | Promoter/ | BSP |
| | | | | Exon1 | (Treated) |
| 29 | Forward | GTTTTGATTAATGTGGGTTGAATTTA | 753 bp | NC_000001.10 | BSP |
| | | | | | (Treated) |
| 30 | Forward | CAGCCTGAAAGGATCCTCCTCAGC | | | BSP |
| | | | | | (Genomic) |
| 31 | Reverse | GCTCTGATCAATGTGGGCTGAATTCA | | | BSP |
| | | | | | (Genomic) |
| 44 | Forward | TAGACCAGTCCGTGATCAAGAAAGT | 310 bp | NM_178548.3 | Expression |
| 45 | Reverse | AGGTTGAGCCCAATCTTCTCTAAC | bp 747-1056 | Exon 3-5 | Expression |
| 46 | Forward | CACCTACTCCGCCATGGAG | 1331 bp | NM_178548.3 | cDNA |
| 47 | Reverse | GTGGGAGAAGCAGTTATTTCCG | CDS | Exon 1-7 | cDNA |

DKK4

| | | | | | |
|---|---|---|---|---|---|
| 32 | Forward | ATATTAGAAAGGCAGCTTGATGAG | 206 bp | NM_014420.2 | Expression |
| 33 | Reverse | TTACAAATTTTCGTCCAAAAATGAC | bp 406-587 | Exon 3-4 | |
| 34 | Forward | GAAAGGGATGAAGCAGAAGTTTTA | 1 kb | NC_000008.10 | Luciferase |
| 35 | Reverse | GTCGTCTGTTTGTCACTGCTTTT | 2 put. AP2 | Promoter | |
| | | | BS | | |
| 36 | Forward | CTCCCAAAGTGCTGGGATTA | 2 kb | NC_000008.10 | Luciferase |
| 37 | Reverse | GCACGTCGTCTGTTTGTCAC | 4 put. AP2 | Promoter | |
| | | | BS | | |
| 38 | Forward | TTTAAGCGGTTGGGATTTTG | 500 bp | NC_000008.10 | Luciferase |
| 39 | Reverse | TAACCAGATGTGCCTCCTCC | 0 put. AP2 | Promoter | |
| | | | BS | | |
| 40 | Forward | TTCGCCTGTGTATATTGCCA | 114 bp | NC_000008.10 | ChIP 1 |
| 41 | Reverse | GATAAAGGAAAGAGCCCCCA | 2 put. AP2 | Promoter | |
| | | | BS | | |
| 42 | Forward | TAAGCGGTTGGGATTTTGAC | 300 bp | NC_000008.10 | ChIP 2 |
| 43 | Reverse | GGCAGAGCAGGATGTCTGTA | 2 put. AP2 | Promoter | |
| | | | BS | | |

ACTB

| | | | | | |
|---|---|---|---|---|---|
| 48 | Forward | TGGTGATGGAGGAGGTTTAGTAAGT | 132 bp | NG_007992.1 | Methylight |
| 49 | Reverse | AACCAATAAAACCTACTCCTCCCTTAA | | | |
| 50 | Probe | ACCACCACCCAACACACAATAACAAACACA | 3′TAMRA- | | |
| | | | 5′FAM | | |

TFAP2A

| | | | | | |
|---|---|---|---|---|---|
| 51 | Forward | CTCGATCCACTCCTTACCTCAC | 396 bp | NM_003220.2 | Expression |
| 52 | Reverse | CCTGCAGGCAGATTTAATCCTA | bp 693-1088 | Exon 4-7 | |

TFAP2B

| | | | | | |
|---|---|---|---|---|---|
| 53 | Forward | AATGGAAGACGTCCAGTCAGTT | 431 bp | NM_003221.3 | Expression |
| 54 | Reverse | AGTGAACAGCTTCTCCTTCCAC | bp 691-1121 | Exon 2-6 | |

TFAP2C

| | | | | | |
|---|---|---|---|---|---|
| 55 | Forward | GATCAGACAGTCATTCGCAAAG | 388 bp | NM_003222.3 | Expression |
| 56 | Reverse | CAAAGTCCCTAGCCAAATGAAC | bp 808-1195 | Exon 3-6 | |

TFAP2D

| | | | | | |
|---|---|---|---|---|---|
| 57 | Forward | GCCAAGGTGGAGTGATAAGAAG | 392 bp | NM_172238.3 | Expression |
| 58 | Reverse | GGCAAGATGTTCTCCTACTGCT | bp 1087-1478 | Exon 3-6 | |

[symbol]

TABLE 4
Characteristics of patients with colorectal cancer screened with
MethyLight for TFAP2E methylation (discovery cohort).

| | | Positive | Negative | |
|---|---|---|---|---|
| | | for TFAP2E | for TFAP2E | |
| | | methylation | methylation | p- |
| | Total | (cutoff 30%) | (cutoff 30% | value |
| Patients | 74 | 38 | 36 | — |
| Age | | | | 0.23 |
| mean +/− SD | 67.2 +/− 9.7 | 69.6 +/− 9.2 | 64.8 +/− 10.2 | |
| range | 37-93 | 55-93 | 37-83 | |
| Gender | | | | 0.81 |
| male | 43 (58%) | 23 (60%) | 20 (55%) | |
| female | 31 (42%) | 15 (40%) | 16 (45%) | |
| Tumor | | | | >0.99 |
| Type | 16/64 | 9/34 (27%) | 7/30 (23%) | |
| Mucinous | (25%) | | | |
| Grading | | | | 0.36 |
| G0-G1 | 59/64 | 30/34 (88%) | 29/30 (97%) | |
| G2-G3 | (92%) | 4/34 (11%) | 1/30 (3%) | |
| | 5/64 (8%) | | | |
| Localisation | | | | 0.25 |
| Rectum | 19/64 | 8/34 (24%) | 11/30 (37%) | |
| Sigma | (30%) | 9/34 (26%) | 10/30 (33%) | |
| Colon | 19/64 | 17/34 (50%) | 9/30 (30%) | |
| | (30%) | | | |
| | 26/64 | | | |
| | (40%) | | | |
| Staging | | | | 0.40 |
| T1-T2 | 17/64 | 11/34 (32%) | 6/30 (20%) | |
| T3-T4 | (27%) | 23/34 (68%) | 24/30 (80%) | |
| | 47/64 | | | |
| | (73%) | | | |
| Lymph | | | | >0.99 |
| Nodes | 26/64 | 14/34 (41%) | 12/30 (40%) | |
| N0 | (41%) | 20/34 (59%) | 18/30 (60%) | |
| N1-N2 | 38/64 | | | |
| | (59%) | | | |
| Metastasis | | | | >0.99 |
| M0 | 59/64 | 31/34 (91%) | 28/30 (93%) | |
| M1 | (92%) | 3/34 (9%) | 2/30 (7%) | |
| | 5/64 (8%) | | | |

### EXPERIMENTS AND EXAMPLES

While the present invention has been described with specificity in accordance with certain of its preferred embodiments, the following example serve only to illustrate the invention and are not intended to limit the invention within the principles and scope of the broadest interpretations and equivalent configurations thereof.

Patients and Tissue Samples

Samples were obtained from patients undergoing colorectal cancer surgery or chemotherapy at the University Hospitals in Munich, Mannheim, Bochum, Berlin and Dresden (all Germany). Tissues were obtained during resection of the primary tumor or by biopsy. Samples were either frozen tissue, formalin-fixed, paraffin-embedded (FFPE) and histology was verified by experienced pathologists (CR, AT; RL, PS). Response to chemotherapy was assessed by RECIST criteria, response to chemoradiotherapy by histology as previously described. Informed consent was obtained prior to enrollment in the study and the study was approved by the Human Subjects Committee of the Technische Universität München.

Cell Culture and 5-aza-cytidine Treatment

Colon cancer cell lines LOVO and DLD-1 were obtained from the Deutsche Sammlung von Mikroorganismen und Zellkulturen GmbH (Braunschweig, Germany). SW480, HT-29, HCT-116 and CACO-2 colon cancer cell lines were obtained from ATCC (Washington, D.C., USA). Cells were cultured in 90% DMEM medium supplemented with 10% fetal bovine serum and 2 mM L-glutamine (Invitrogen, Karslruhe, Germany). CACO-2 cells were maintained in 80% DMEM with 20% FBS. Cells were seeded at a density of 1×10⁶ cells/60-mm dish, after twenty-four hours incubated with 5-aza-cytidine (final concentration 10 μmol/L; Sigma, Munich, Germany) or the same amount of DMSO as control. Culture medium was changed daily for 3 days with recurrent 5-aza-2-deoxycytidine additions. After 3 days, the cells were harvested for total RNA extraction with the RNeasy Total RNA Mini Kit (Qiagen, Hilden, Germany).

Primer Design

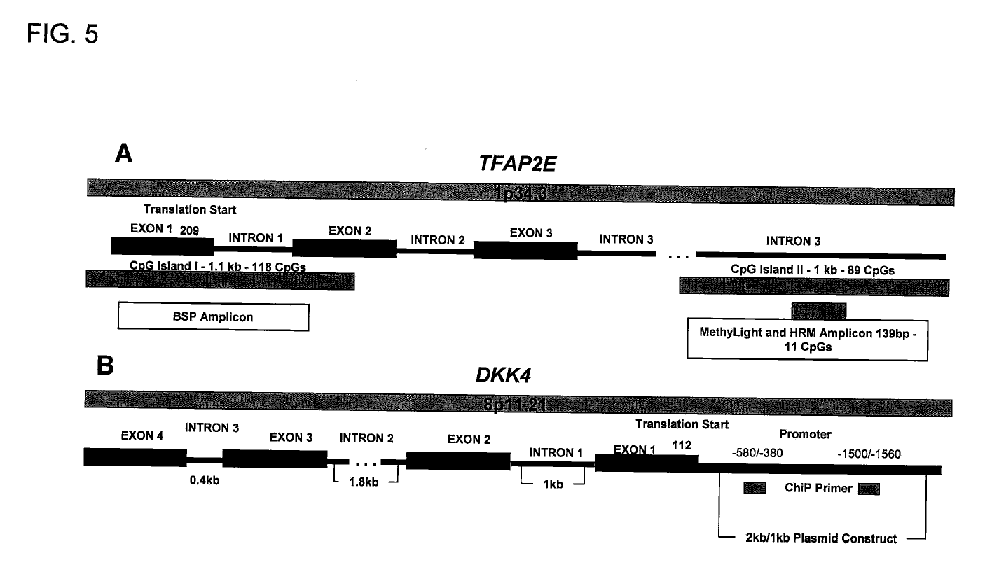

Primers for methylated TFAP2E covered CpGs in the second CpG island (FIG. 5). Results from the MethyLight assays were confirmed using bisulfite sequencing (in selected cases) and Methylation-Sensitive High Resolution Melting (MS-RHM) Analysis. To avoid assay deviations due to limited DNA content/degradation in the clinical samples, only such samples were included in the analysis that showed sufficient expression of the reference gene (ACTB), reflected by a Cp of >37 cycles, and value deviations of >1 cycle in the replicate measurements. Since MS-HRM tends to be more sensitive then classical MSP or MethyLight, MS-HRM was also used with the same primers. Primer sequences are listed in Table 2. MethyLight and MS-HRM conditions were as follows: 10 min 95° C. for activation and then 50 cycles consisting of 15 sec 95° C. and 30 sec 60° C. and 10 sec 72° C. (data acquisition at this step), for MS-HRM this was followed by 1 min 95° C., 1 min 40° C., 1 sec 65° C. and then continuous melting to 95° C.

DNA and RNA Extraction

The RNA and DNA extraction from cell lines or 10-25 mg of frozen tissue samples was carried out using the RNeasy Total RNA Mini Kit or the QIAmp DNA Mini Kit (Qiagen, Hilden, Germany) respectively, using the manufacturer's instructions.

RT-PCR and MethyLight/MS-HRM analysis

Genomic DNA was treated with the Epitect Bisulfite Conversion Kit (Qiagen, Hilden, Germany) according to the manufacturer's instructions and then analyzed using the MethyLight technique or MS-HRM as previously described. Cutoff values giving the best discrimination between non-neoplastic mucosa and tumor samples were determined by receiver operating characteristic (ROC) analysis. The expression analysis of the mRNA was performed by RT-PCR with the Verso cDNA Kit (Thermo Fischer, Darmstadt, Germany) according to the manufacturer's instructions.

Expression Microarray and Verification of Target Candidates

pTFAP2E SW480 clones and SW480 pTarget clones were used for global expression analysis using a Human Gene 1.0 ST Expression Array (Affymetrix, High Wycombe, United Kingdom) according to the manufacturer's protocol. Genes with a more than 3-fold expression change were verified by quantitative RT-PCR (see table 3).

Reporter and Expression Vectors

Full length TFAP2E coding sequence was amplified from SW480 cells and cloned with and without FLAG epitope (pTFAP2E and pTFAP2eFlag) into the pTarget vector (Promega, Mannheim, Germany) according to the manufacturer's instructions. The DKK4 promoter sequence was also amplified and cloned into pGL3 basic vector (Promega)—pGL3-DKK4-1 kb (insert size: 1 kb, from minus 1 kb of the transcription start site). The pRL-TK reporter plasmid (Promega) was used as an internal control reporter vector. Full length DKK4-CDS (pcDNA3-Dkk4) and a DDK4 promoter plasmid—pGL3-DKK4-2kb (pGL3 basic vector—insert: 2 kb, minus 2000 bp of the transcription start) were obtained from Dr. Kolligs, München.

Generating of Clones with Stable Overexpression of TFAP2E

SW480 clones stably overexpressing TFAP2E and control clones using an empty pTarget vector were obtained after transfection with pTFAP2E and pTFAP2eFlag using Lipofectamine 2000 (Invitrogen, Karslruhe, Germany) according to the manufacturer's instructions. Stable transfectants were selected upon G418 treatment (Invitrogen, Karslruhe, Germany) for 2 weeks. Single colonies were picked and grown further in selective media. TFAP2E expression was assessed by quantitative RT-PCR.

Transient Transfections and Luciferase Assays

SW480, CACO-2 and HT-29 cells were transfected with either pGL3-DKK4-1 kb or pGL3-DKK4-2 kb, and pRL-TK as transfection control (renilla luciferase). Transfections were carried out with Satisfection Transfection Reagent (Agilent, Waldbronn, Germany). After 3 days, cells were harvested and firefly and renilla luciferase activities were measured on a luminometer using the Luciferase Dual Reporter Assay (Promega, Mannheim, Germany). All experiments were repeated independently 3 times and all cells were seeded in triplicate on the 96 well plates.

Chromatin Immunoprecipitation

pTFAP2eFlag SW480 clones were used for ChIP according to the protocol of ChIP assay kit (Upstate Biotechnology Inc, New York, USA) with anti-FLAG antibody (Sigma, Munich, Germany). Primer sequences of the DKK4 promoter region flanking putative AP-2 protein binding sites are given in table 2.

Stress Resistance and Cell Survival Assays After Drug Exposure

Cells were seeded in 96 well plates (20.000 per well) and cell proliferation was measured by MTT (Sigma, Munich, Germany) and BrDU (Roche, Penzberg, Germany) assays according to the manufacturer's protocol. For assessment of stress induced apoptosis, cells were seeded in 96 well plates (50.000 per well) and treated with TNF-alpha (100 ng/μl) and chlorhexamide (100 μg/μl) for 4 days. Resistance of cells to chemotherapeutic drugs was investigated after treatment with 5-fluorouracil (50 μg/ml or 38 μM), oxaliplatin (60 μM or 24 μg/ml) or irinotecan (20 μM or 13 μg/ml) and assessed by MTT assay.

Invasion and Migration

Cells were seeded on Transwell Matrigel Chambers with reduced growth factors (BD Biosciences, Heidelberg, Germany) according to the manufacturer's protocol (25.000 cells) in serum free media.

Statistics

Receiver Operating Characteristic analysis was performed in order to determine the optimal methylation (PMR value) cutoff. As result, PMR values above 30% were considered as methylation positive and classified as ‘1’, whereas PMR levels below 30% were classified as ‘0’. Correlations of the methylation event with clinicopathological features, such as primary tumor site, histological grade of differentiation or stage of cancer were assessed by the Fisher's exact test. The Mann-Whitney U test was used to compare the distribution of quantitative data between two independent samples. To consider potential cluster effects by different study centers involved, random effect models were employed for statistical analysis of response probability in relation to PMR. Based on these models, estimates of response ratios (relative risks) were provided with 95% (fixed effect) confidence intervals. All tests were two-sided, and a p-value of <0.05 was considered statistically significant. Statistical calculations were done with Graphpad Instat 3, Graphpad Prism 5 (GraphPad Software, Inc., La Jolla, USA), MIX 1.7 software and the R statistical software v2.9 (R Foundation for Statistical Computing, Vienna, Austria).

Results

Frequent Hypermethylation of TFAP2E in Human Colorectal Cancer

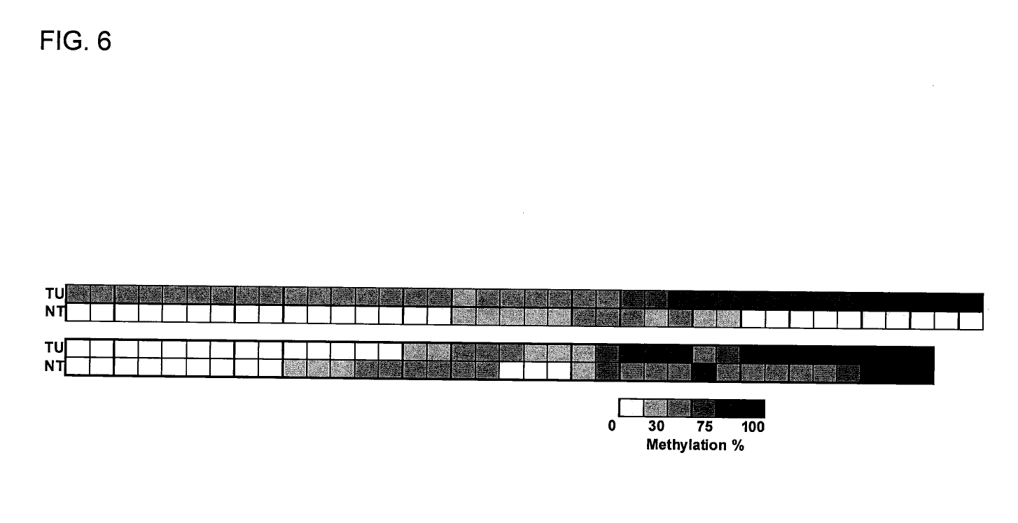

Genomic DNA was obtained from primary CRC and adjacent mucosa from 74 CRC patients (initial cohort, all snap frozen) and was analyzed for TFAP2E methylation. With a cut-off value of 30%, 38/74 patients (51.4%) were classified as methylated (FIG. 6). None of the common clinico-pathological characteristics of CRC patients, including primary tumor site, histological grade of differentiation or stage of cancer, age or gender, correlated with TFAP2E methylation status (Table 4).

Hypermethylation of TFAP2E in CRC is Associated with Decreased Gene Expression

Tumor-mRNA from 28/74 CRCs (initial cohort) was analyzed for TFAP2E expression by RT-PCR. In 12 cases TFAP2E methylation and decreased TFAP2E expression was observed, while 8 cases showed no methylation and strong expression. The remaining 8 cases showed either expression with methylation or no expression without methylation. There was a significant correlation between methylation and expression (Fisher's exact test, p<0.05).

Analysis of TFAP2E expression and Methylation in CRC Cell Lines

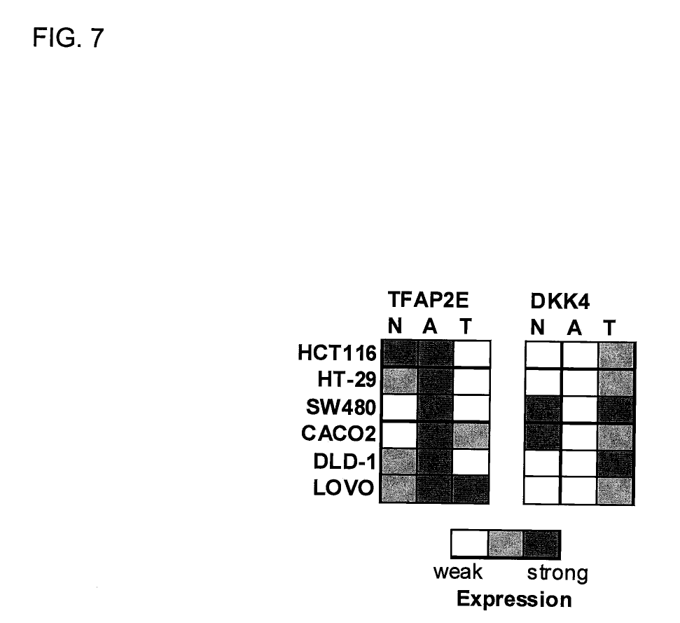

HCT-116, HT-29, DLD-1, LOVO, CACO-2, and SW480 cells were analyzed by RT-PCR for the expression of TFAP2E. While HCT-116, HT-29, DLD-1 and LOVO had constitutive TFAP2E expression, CACO-2 and SW480 cells showed expression after 5-aza-cytidine treatment only. Treatment with Trichostatin A showed no effect on TFAP2E mRNA levels, except for CACO-2 which showed a weak expression after treatment. MethyLight revealed a high degree of TFAP2E methylation in all CRC cell lines, which declined after 5-azacytidine treatment (FIG. 7).

Expression of TFAP2E Affects Migration of Human CRC Cells

Migration of cancer cells was assessed in stable clones of SW480 transfected with pTFAP2E or pTarget (empty vector control), designated SW480-pTFAP2E and SW480-pTarget, respectively. A minor 1.3-fold increase of migrating cells was observed in control versus clones stably overexpressing TFAP2E (p<0.05). In contrast, no differences in proliferation and stress resistance were observed (data not shown).

Identification of DKK-4 as a Downstream Target of TFAP2E

For the identification of potential downstream targets of TFAP2E, SW480-pTFAP2E and SW480-pTarget cell clones were subjected to microarray analysis (Table 3). The verification of these results by quantitative real-time PCR confirmed only DKK4 as a target gene that was significantly down-regulated (mean 6-fold, range 3- to 9-fold) in all stable SW480-pTFAP2E clones versus empty SW480-pTarget vector controls. DKK4 mRNA levels were then analyzed in untransfected CRC cell lines. SW480 and CACO-2 showed strong DKK4 expression that was lost upon re-expression of TFAP2E through 5-azacytidine treatment. All cell lines with basal expression levels of TFAP2E (HCT-116, HT-29, DLD-1, LOVO) showed no DKK4 expression (FIG. 7).

TFAP2E Binds to the DKK-4 Promoter and Represses Expression in Vitro

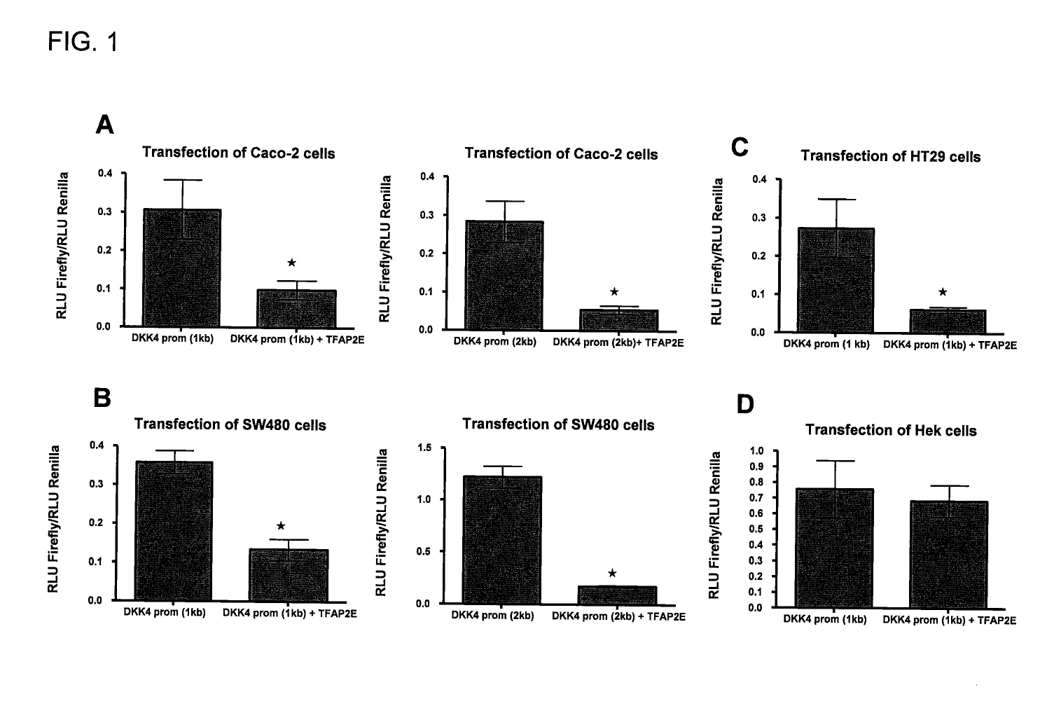

Using the Transcription Element Search System we found 2 putative binding sites for AP-2 proteins within 2kb upstream and 2 sites within 1 kb upstream of the DKK4 mRNA transcription start site (FIG. 5). To analyze binding of TFAP2E to these sites, 1 kb and 2 kb of the DKK4 promoter were cloned into a Luciferase reporter vector (pGL3) and transfected together with pTFAP2E and pRL-TK (internal control) into SW480, CACO-2 (no TFAP2E expression) and HT-29 (weak TFAP2E expression) cells. Luciferase activity was decreased in CACO-2 cells transfected with TFAP2E compared to cells without. TFAP2E transfection, 3-fold with the pGL3-DKK4-1 kb vector and 5-fold with the pGL3-DKK4-2 kb vector (FIG. 1A). SW480 cells showed the same effect with 3- to 7-fold downregulation (7-fold with pGL3-DKK4-2kb vector, FIG. 1B) as well as in HT-29 cells (3-fold with pGL3-DKK4-1 kb vector, FIG. 1C; Wilcoxon test p<0.005). HEK cells showed no difference in luciferase activity with or without TFAP2E transfection. This might be due to the high endogenous TFAP2E expression level (FIG. 1D). As a control experiment, a 500 bp vector construct of the DKK4 promoter flanking, but not including the 2 binding sites present in the pGL3-DKK4-1 kb vector, was transfected into SW480 cells together with TFAP2E-CDS, showing no reduction of luciferase activity and indicating that these sites are indeed crucial for TFAP2E binding (data not shown).

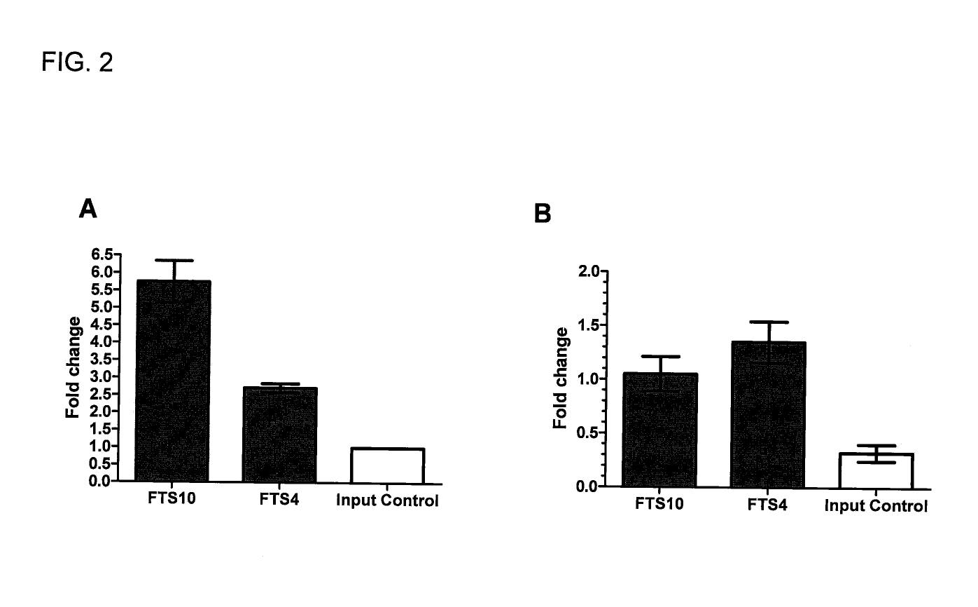

To prove that the TFAP2E protein binds directly to the DKK4 promoter, chromatin immunoprecipitation was performed with cells of two stable SW480 pTFAP2eFlag clones. The recovered DNA of the input control, negative control and the SW480 pTFAP2eFlag clones was used for real time PCR with two primer pairs flanking 4 putative AP-2 protein binding sites in the DKK4 promoter (two sites 2 kb upstream and two sites 1 kb upstream of the DKK4 transcription start site), respectively. While the negative control showed no amplification, both clones showed a 4-fold higher amplification for the two sites 2 kb upstream (FIG. 2A) and 1.5-fold higher for the two sites 1 kb upstream (FIG. 2B), indicating a direct binding of TFAP2E protein to the DKK4 promoter.

Inverse TFAP2E and DKK4 Expression Pattern in Colorectal Cancer Patients

Tumor-mRNA from 28 CRCs (out of 74 CRCs) was used for expression analysis. In 11 cases the DKK4 expression pattern (strong expression) was inversely related to the expression of TFAP2E (decreased expression) and vice versa in 10 cases (no DKK4 expression and TFAP2E expression), 7 cases showed comparable expression levels in both tissue types. The inverse correlation between TFAP2E and DKK4 expression proved to be significant (Fisher's exact test, p<0.01).

Expression of TFAP2E Affects Resistance to 5-FU in Human CRC Cells

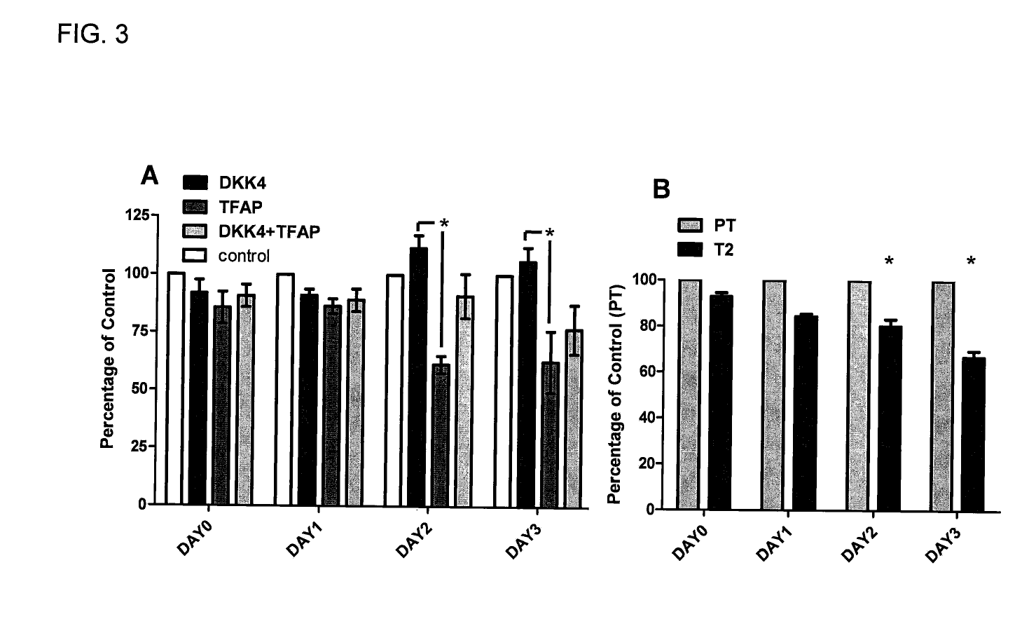

Since DKK4 has been implicated in the responsiveness to chemotherapy and the present invention findings point to DKK4 as a potential downstream target of TFAP2E, the role of TFAP2E and DKK4 in mediating drug resistance in CRC cancer cells in vitro was assessed. SW480 cells were transiently transfected with pTFAP2E or pcDNA3-Dkk4 and treated with oxaliplatin, irinotecan or 5-FU. Surviving cells were measured by MTT assays. All experiments were performed in triplicates. SW480-pTFAP2E transfected cells showed a significant decrease (about 20% compared to controls) and SW480 pcDNA3-Dkk4 transfected cells showed a significant increase in survival (about 10% compared to controls) when treated with 5-FU after 2 days (Kruskal-Wallis test, p<0.01) (FIG. 3A). Cells transfected with both pTFAP2E and pcDNA3-Dkk4 exhibited intermediate responsiveness. In addition, these results were confirmed in SW480-pTFAP2E clones and SW480-pTarget vector controls which were treated with 5-FU (Mann-Whitney U test, p<0.001, FIG. 3B).

TFAP2E Methylation Predicts Resistance to Cancer Chemotherapy

Next, TFAP2E methylation was assessed in 4 different cohorts of colorectal cancer patients undergoing chemotherapy or chemoradiotherapy with a 5-FU based regimen in order to analyse chemoresistance in vivo.

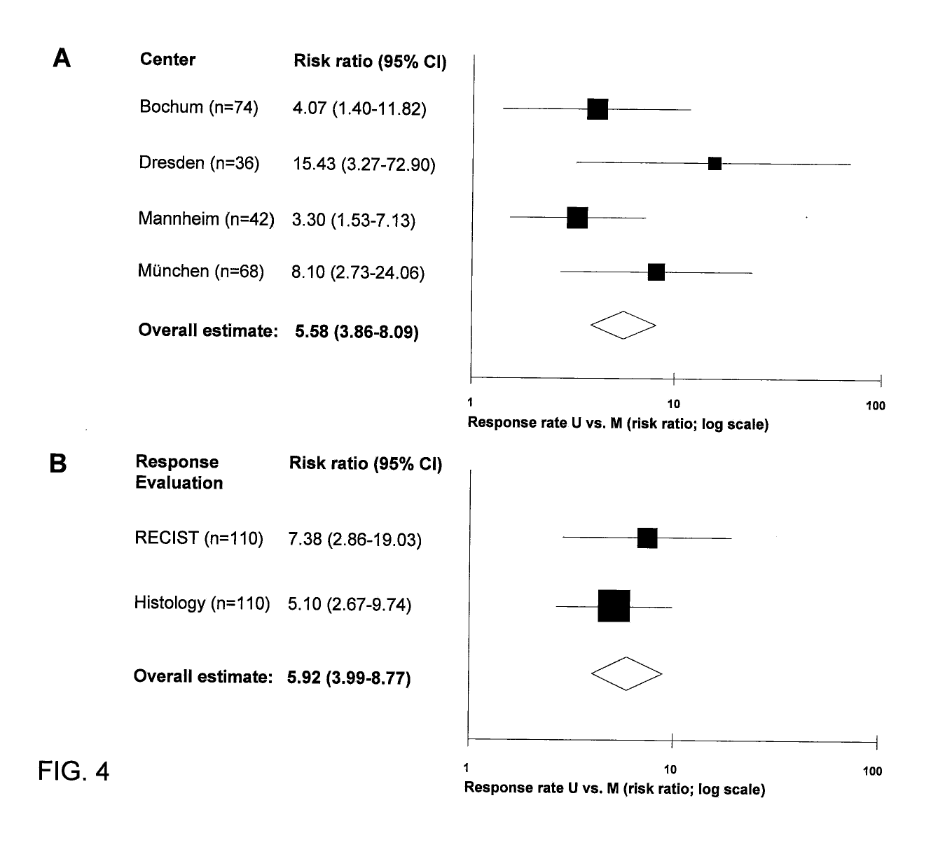

Cohort I (Bochum) included 76 patients who were enrolled in a prospective trial comparing the oral fluoropyrimidine capecitabine with intravenous 5-FU together with oxaliplatin (CAPDX versus FUFOX) in metastatic CRC patients. Of the 76 samples, 74 cases yielded enough DNA for methylation analysis and clinical response data was available for all of them. Of these patients, 36 (49%) where classified as responders and 38 (51%) as nonresponders according to RECIST criteria. TFAP2E methylation was found in 3 responders and 17 nonresponders (23%); 33 (45%) responders and 21 nonresponders (28%) were unmethylated. TFAP2E methylation was significantly more frequent in non-responders (Fisher's exact test p<0.0005; Table 1 and FIG. 4A).

Cohort II (Dresden) consisted of 44 samples from patients with metastatic CRCs undergoing 5-FU based polychemotherapy (FOLFIRI/FOLFOX). Of these, 43 yielded DNA for methylation analysis, clinical response data was available for 39 patients but 3 patients stopped chemotherapy due to intolerable toxicity and were excluded from the analysis. Of these remaining 36 patients, 14 (39%) were classified as responders and 22 (61%) were classified as nonresponders according to RECIST criteria. TFAP2E methylation was observed in all 22 nonresponders and 1 responder (64%), 13 (36%) responders showed no methylation. The 3 patients who stopped chemotherapy due to toxicity (excluded from the response analysis) showed also no TFAP2E methylation. Again, the correlation between TFAP2E methylation and treatment response was significant (Fisher's exact test p<0.0001; Table 1 and FIG. 4A).

For cohort III (Mannheim), DNA from 50 patients with rectal cancer undergoing chemoradiotherapy with 5-FU in combination with Irinotecan, Cetuximab was analysed with response based on histological evaluation. 49 samples yielded DNA, but for only 42 patients matching clinical data was available. Of these, 25 (60%) responders and 17 (40%) nonresponders were classified by histological criteria. TFAP2E methylation was found in 5 (12%) responders and 14 (33%) nonresponder, while 20 (48%) responders and 3 nonresponders were unmethylated. The correlation between TFAP2E methylation and treatment response was significant (Fisher's exact test p<0.0001; Table 1 and FIG. 4A) in this cohort as well.

Cohort IV (München) consisted of 70 patients with primary rectal cancer who underwent 5-FU based chemoradiotherapy (intravenous 5-FU and 45.0 Gy). Treatment response was determined again by histological response criteria. From 70 samples, 69 yielded enough DNA and clinical response data were available in 68 cases. Of the 68 patients analysed, 32 (47%) were classified as responders and 36 (53%) as nonresponders. TFAP2E methylation was observed in 3 (4%) responders and 28 (41%) nonresponders, while 29 (43%) responders and 8 (12%) nonresponders showed no methylation. The correlation between TFAP2E methylation and treatment response was also significant in this cohort (Fisher's exact test p<0.0001; Table 1 and FIG. 4A).

The association of methylation and treatment response was apparent in all four patient series (higher response for non-methylated patients), which is shown by a significant difference in response rates. A substantial effect size was estimated for the pooled cohort data (FIG. 4A) with a response ratio of 5.58 (95% CI: 3.86-8.09). When the cohorts were pooled according to response evaluation criteria (histology or RECIST), the overall estimated effect was even stronger with a response ratio of 5.92 (95% CI: 3.99-8.77; FIG. 4B).

The above results show that the TFAP2E gene is frequently hypermethylated in cancers from CRC patients and in these cancers TFAP2E mRNA expression is either lost or downregulated. Loss or downregulation of TFAP2E expression was also observed in CRC cell lines but was restored by the treatment with the methylation inhibitor 5-azacytidine. Further, the functional role of TFAP2E in CRC was assessed. After stable transfection of CRC cells in vitro, no difference in apoptosis or proliferation was observed, however, a significant difference in cell migration was observed after TFAP2E overexpression.

In order to characterize the down-stream targets of TFAP2E microarray analysis of TFAP2E overexpressing cells was performed and found DKK4 to be a potential target gene which was significantly down regulated via TFAP2E.

DKK4 is a member of the dickkopf family, comprising various antagonists of WNT-signalling by binding to WNT-coreceptors LRP5/6. DKK4 overexpression has been observed in the colon mucosa of patients with colitis. However, the precise role of DKK4 in the colon mucosa and its contribution to carcinogenesis is so far unknown. Recently, DKK4 was implicated in 5-FU resistance in CRC cell lines. The present invention in vitro data from cell lines treated with 5-FU, oxaliplatin and irinotecan confirmed this observation. DKK4 overexpression led to increased 5-FU chemoresistance in CRC cell lines, while introduction of TFAP2E was associated with an increased sensitivity towards 5-FU treatment. According to the above results, there exists a strong association between TFAP2E methylation and lack of chemotherapy response in the tumor. Fixed effect model analysis of the pooled cohort data revealed an estimated 6-fold higher response probability for non-methylated patients. Interestingly, the correlation was observed in primary rectal cancers and metastatic colorectal cancers, independent of the treatment with 5-FU based chemotherapy or chemoradiotherapy. Also, assessment of response in these cohorts, either by standard RECIST criteria or by histological response, did not influence this strong association, indicating that TFAP2E methylation is valuable for response prediction in either setting.

What is claimed is:

1. A method of predicting, prognosing, and/or monitoring therapeutic efficacy of cancer therapy on a subject having malignant tumor and/or cell proliferative disorder comprising:
c) determining the methylation and/or expression levels of TFAP2E in a biological sample isolated from said subject; and
d) predicting, prognosing, and/or monitoring therapeutic efficacy of cancer therapy on said subject based on step a),
wherein hypermethylation and/or decreased expression of TFAP2E is indicative of resistance to cancer therapy, while hypomethylation and/or increased expression of TFAP2E is indicative of sensitivity to cancer therapy.

2. The method according to claim 1, wherein said predicting, prognosing, and/or monitoring is determined by detecting the presence, absence or amount of CpG methylation within TFAP2E or sequence thereof.

3. The method according to claim 1, wherein said expression level is determined by detecting the presence, absence or level of mRNA transcribed from TFAP2E.

4. The method according to claim 3, wherein said expression level is determined by detecting the presence, absence or level of a polypeptide encoded by TFAP2E or sequence thereof.

5. A method according to claim 1, wherein said malignant tumor and/or cell proliferative disorder is colorectal, pancreatic, gastric, head and neck, breast, non-small-cell lung, leukaemia, and/or gynaecologic cancer.

6. A method according to claim 1, wherein said cancer therapy comprises chemotherapy, polychemotherapy, chemoradiotherapy, treatments with chemotherapeutic agents, treatments with antimetabolites, pyrimidine antagonists, cytarabine, decitabine, floxuridine, capecitabine, or Gemcitabine and/or combination thereof.

7. A method according to claim 1, wherein said cancer therapy is 5-Flurouracil based chemotherapy or polychemothrapies.

8. A method according to claim 2, comprising contacting genomic DNA isolated from a biological sample obtained from said subject with at least one reagent, or series of reagents that distinguishes between methylated and non-methylated CpG dinucleotides within at least one target region of the genomic DNA, wherein the target region comprises, or hybridizes under stringent conditions to a sequence of contiguous nucleotides of SEQ ID NO: 1, wherein said contiguous nucleotides comprise at least one CpG dinucleotide sequence.

9. A method according to claim 8, further comprising:
a. contacting the treated genomic DNA, or the treated fragment thereof, with an amplification enzyme and at least one oligonucleotide comprising a contiguous sequence of nucleotides that is complementary to, or hybridizes under moderately stringent or stringent conditions to a sequence selected from the group consisting of SEQ ID NOs: 2-5, and complements thereof, wherein the treated genomic DNA or the fragment thereof is either amplified to produce at least one amplificate, or is not amplified; and
b. determining, based on a presence, absence or amount of, or on a property of said amplificate, the methylation state or level of at least one CpG dinucleotide of SEQ ID NO: 1, or an average, or a value reflecting an average methylation state or level of a plurality of CpG dinucleotides of SEQ ID NO: 1, whereby predicting, prognosing, and/or monitoring of said subject is afforded.

10. A method according to claim 8, wherein treating the genomic DNA, or the fragment thereof, comprises use of a reagent selected from the group comprising of bisulfite, hydrogen sulfite, disulfite, and combinations thereof.

11. A method according to claim 1, wherein the biological sample obtained from the subject is selected from the group comprising metastatic cells, cell lines, histological slides, biopsies, paraffin-embedded tissue, frozen tissue, formalin fixed tissue, body fluids, stool, colonic effluent, urine, blood plasma, blood serum, whole blood, isolated blood cells, cells isolated from the blood and combinations thereof.

12. A method according to claim 2, further comprising:
a. digesting the genomic DNA, or a fragment thereof, isolated from a biological sample obtained from said subject with one or more methylation sensitive restriction enzymes;
b. contacting the DNA restriction enzyme digest of a), with an amplification enzyme and at least one oligonucleotide suitable for the amplification of a sequence comprising at least one CpG dinucleotide of SEQ ID NO: 1;
c. determining, based on a presence, absence or class of an amplificate the methylation state or level of at least one CpG dinucleotide of at least one sequence selected from the group consisting of SEQ ID NO: 1, and therefrom predicting, prognosing, and/or monitoring of said subject is afforded.

13. A kit for predicting, prognosing, and/or monitoring therapeutic efficacy of cancer therapy in a subject having malignant tumor or cell proliferative disorder who has undergone or is undergoing chemotherapy and/or chemoradiotherapy, characterized in that said kit comprises (a) one or more reagents to convert unmethylated cytosine bases to uracil or to anther base that is detectably dissimilar to cytosine in terms of hybridization properties (b) at least one oligonucleotide or polynucleotide able to hybridize under stringent or moderately stringent conditions to at least one sequence selected from the group consisting of SEQ ID NOs.: 2-5 in order to detect the presence, absence or amount of CpG methylation within TFAP2E, c) containers suitable for containing the said one or more reagents and the biological sample of the patient; and optionally (d) instructions for use.

14. A kit for predicting, prognosing, and/or monitoring therapeutic efficacy of cancer therapy in a subject having malignant tumor or cell proliferative disorder who has undergone or is undergoing chemotherapy and/or chemoradiotherapy, characterized in that said kit comprises (a) a methylation sensitive restriction enzyme reagent; (b) a container suitable for containing the said reagent and the biological sample of the patient; (c) at least one set of oligonucleotides one or a plurality of nucleic acids or peptide nucleic acids which are identical, are complementary, or hybridize under stringent or highly stringent conditions to a sequence of SEQ ID NO: 1 in order to detect the presence, absence or amount of CpG methylation within TFAP2E; and optionally (d) instructions for use.

15. The use of a method according to claims 1 to 12, and/or a kit according to claim 13 or 14 in predicting, prognosing, and/or monitoring therapeutic efficacy of cancer therapy in a subject having malignant tumor or cell proliferative disorder.
